(19) United States
(12) Patent Application Publication
(10) Pub. No.: US 2026/0176286 A1
(43) Pub. Date: Jun. 25, 2026

(54) DIHYDROARTEMISININ DERIVATIVES AND METHODS OF MAKING AND USE THEREOF

(71) Applicant: Laboratory for Synthetic Chemistry and Chemical Biology Limited, Hong Kong (CN)

(72) Inventors: Chi-Ming CHE, Hong Kong (CN); Yiu Chung Gulice LEUNG; Mei-Ling RUAN, Hong Kong (CN)

(21) Appl. No.: 19/416,621

(22) Filed: Dec. 11, 2025

Related U.S. Application Data
(60) Provisional application No. 63/737,111, filed on Dec. 20, 2024.

(51) Int. Cl. C07D 493/20; A61K 31/357; A61K 45/06; A61P 35/00

(52) U.S. Cl. CPC C07D 493/20; A61K 31/357; A61K 45/06; A61P 35/00

(57) ABSTRACT

Dihydroartemisinin (“DHA”) derivatives that contain a stereospecific C-10 ether linkage and an aryl pharmacophore moiety are disclosed. In some forms, the DHA derivatives possess anticancer properties that are comparable to or better than the parent DHA and/or an existing anticancer drug (e.g., Cisplatin), and are useful in anticancer treatments (such as ovarian cancer and colon cancer). Methods of synthesizing the DHA derivatives that are cost effective are disclosed. Methods of using the DHA derivatives for treating a cancer, such as ovarian cancer and/or colon cancer, in a subject in need thereof are also disclosed.

### CROSS-REFERENCE TO RELATED APPLICATIONS

This application claims the benefit of and priority to U.S. Provisional Application No. 63/737,111 filed Dec. 20, 2024, the entire content of which is incorporated herein by reference in its entirety.

### FIELD OF THE INVENTION

This invention is generally in the field of dihydroartemisinin derivatives and methods of synthesis and uses thereof.

### BACKGROUND OF THE INVENTION

Artemisinin (ART) isolated from the traditional Chinese medicinal herb Artemisia annua, and its derivatives are clinically used as anti-malarial drugs worldwide, and are regarded as one of the greatest recent clinical successes arising from traditional medicine. Dihydroartemisinin (DHA) as a semisynthetic derivative of artemisinin, is the active metabolite of all artemisinin compounds such as artemisinin, artesunate, and artemether. However, the use of DHA and its derivatives are limited to antimalarial in the market.

There remains a need to develop DHA derivatives that can be used in other diseases (such as cancers) and optionally have improved safety profile, and their synthesis thereof.

Therefore, it is the object of the present invention to provide DHA derivatives that possess anticancer properties.

It is a further object of the present invention to provide methods of synthesizing the DHA derivatives.

It is a further object of the present invention to provide methods of using the DHA derivatives as anticancer agents.

### SUMMARY OF THE INVENTION

Dihydroartemisinin derivatives (also referred to herein as “compounds” or “DHA derivatives”) have been developed. The DHA derivatives disclosed herein contain a stereospecific C-10 ether linkage and an aryl pharmacophore moiety.

In some forms, the DHA derivatives possess anticancer properties that are comparable to or better than the parent DHA and/or an existing anticancer drug (e.g., Cisplatin, also referred to as “DDP”), and are useful in anticancer treatments (such as ovarian cancer and colon cancer). For example, the DHA derivatives show cytotoxicity against cancer cells (e.g., as indicated by its IC₅₀ values), such as ovarian cancer cells (e.g., A2780 cells and/or A2780CisR cells) and/or colon cancer cells (e.g., HCT-116 cells), similar to (e.g., concentration in μM on the same order of magnitude) or better than (e.g., concentration in μM that is at least 1 order of magnitude lower) that of DHA and/or Cisplatin. In some forms, the DHA derivatives show a cytotoxicity against A2780 cells that is similar to a cytotoxicity against A2780CisR cells, and thereby overcoming Cisplatin resistance in ovarian cancer cells.

In some forms, the DHA derivatives can have the structure of Formula Ia or Ib:

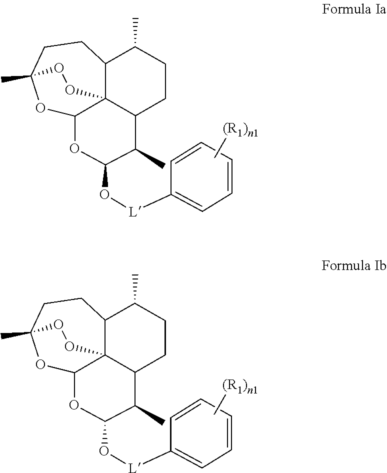

wherein: (i) R₁ can be an electron withdrawing group; (ii) n1 can be an integer from 0 to 5, from 1 to 5, from 1 to 4, from 1 to 3, or 1 or 2, such as 1; and (iii) L′ can be

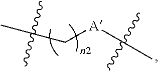

(a) n2 can be an integer from 1 to 6, from 1 to 5, from 1 to 4, from 1 to 3, or 1 or 2; (b) A′ can be a bond or

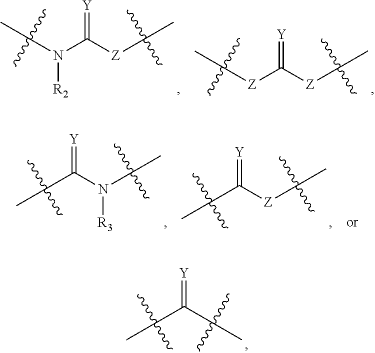

each occurrence of Y and Z are independently O or S; and (c) R₂ and R₃ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

In some forms, A′ can be a bond or

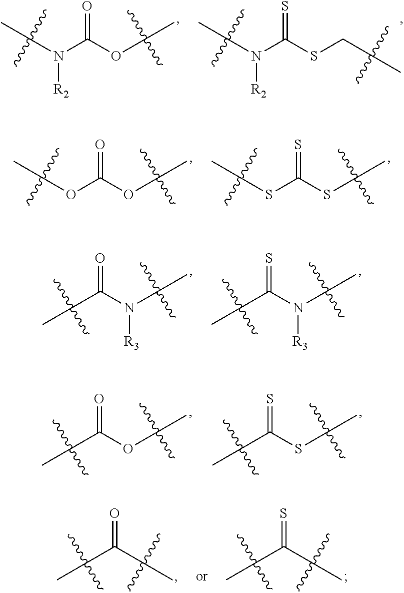

and R₂ and R₃ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

It is understood that the linker L′ can attach to the oxygen at C-10 position via either side. When A′ is

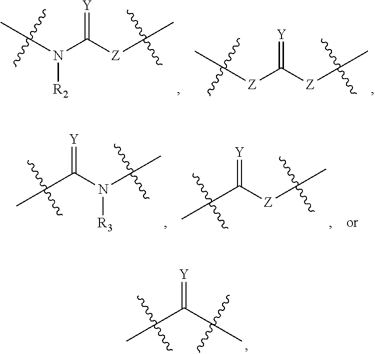

each occurrence of Y and Z are independently O or S (such as

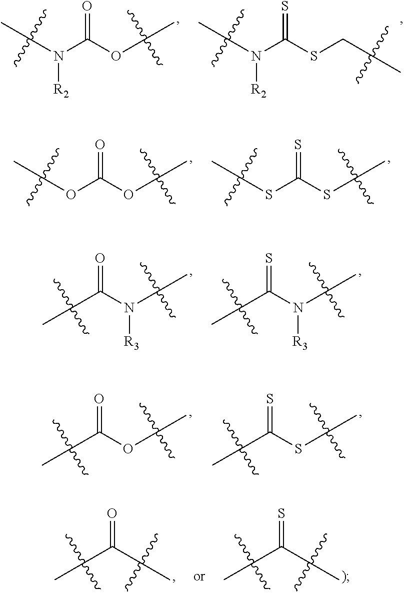

it is understood that either side of the moiety can attach to the phenyl group. For example, when A′ is

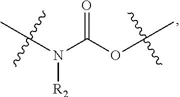

L′ can be either

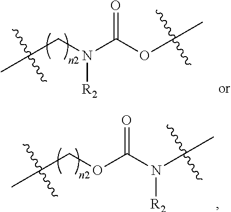

and L′ can attach to the C-10 oxygen atom via the alkylene or via the oxygen or nitrogen atom at the terminus of the linker.

In some forms, the DHA derivatives can have the structure of Formula IIa or IIb:

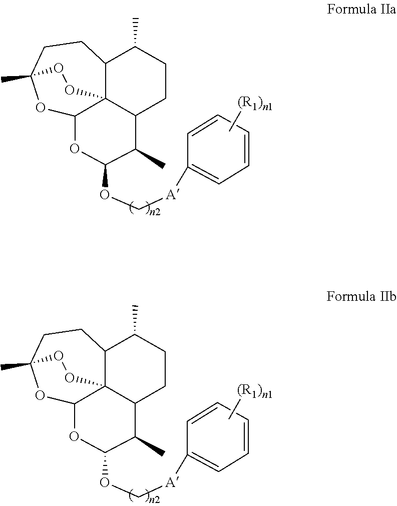

wherein: (i) R₁ can be an electron withdrawing group; (ii) n1 can be an integer from 0 to 5, from 1 to 5, from 1 to 4, from 1 to 3, or 1 or 2, such as 1; (iii) n2 can be an integer from 1 to 6, from 1 to 5, from 1 to 4, from 1 to 3, or 1 or 2; (iv) A′ can be a bond or

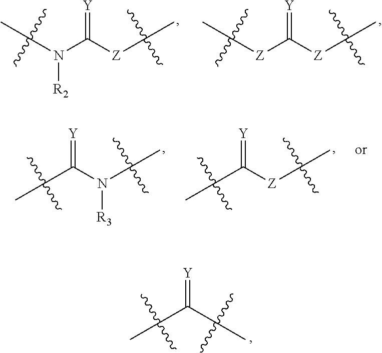

each occurrence of Y and Z are independently O or S (such as

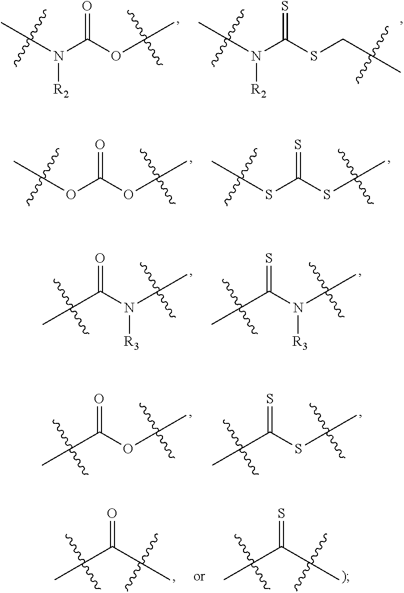

wherein R₂ and R₃ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

In some forms, the DHA derivatives can have the structure of Formula IIa. In some forms, A′ can be a bond,

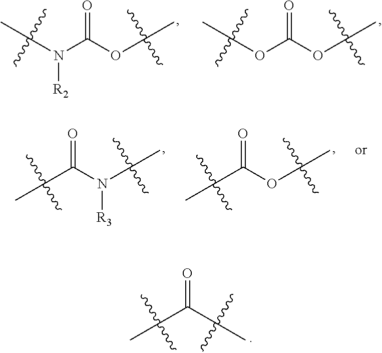

In some forms, A′ can be a bond,

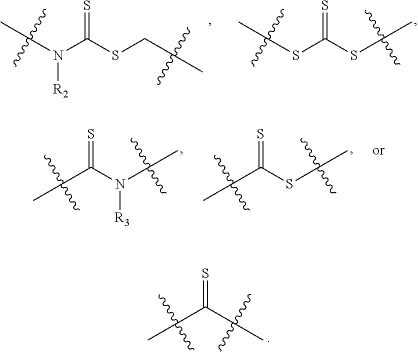

In some forms, A′ can be a bond,

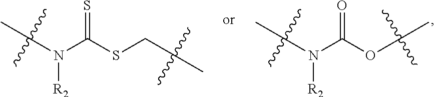

wherein R₂ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

In some forms, A′ can be a bond.

In some forms, the DHA derivatives can have the structure of Formula IIIa, IIIb, IIIc, or IIId:

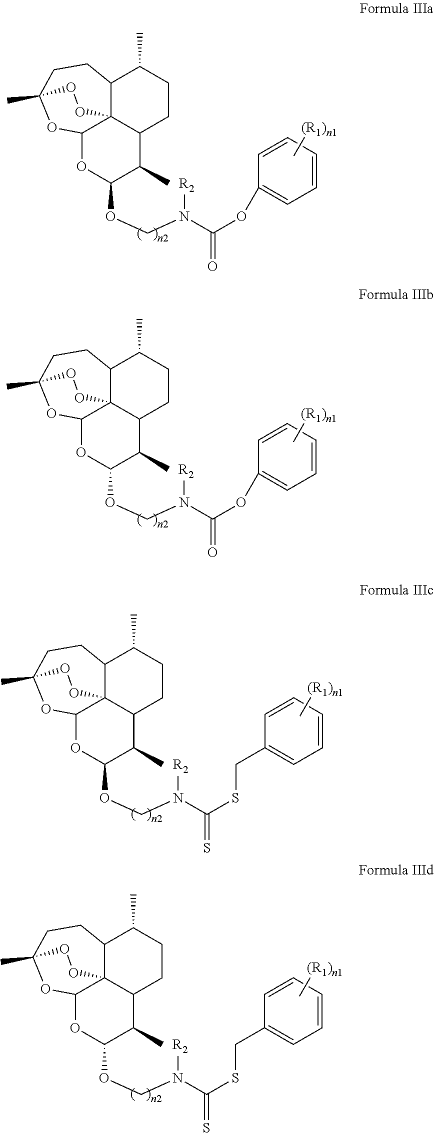

wherein: (i) R₁ can be an electron withdrawing group; (ii) n1 can be an integer from 0 to 5, from 1 to 5, from 1 to 4, from 1 to 3, or 1 or 2, such as 1; (iii) n2 can be an integer from 1 to 6, from 1 to 5, from 1 to 4, from 1 to 3, or 1 or 2; and (iv) R₂ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

In some forms, R₂ and R₃ can be independently an alkyl, such as an unsubstituted C1-C6 alkyl, a substituted C1-C6 alkyl, an unsubstituted C1-C5 alkyl, an unsubstituted C1-C4 alkyl, an unsubstituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol (e.g., —SH), an alkoxy (e.g., —O-alkyl), a carboxylic acid (—COOH), a ketone (e.g., —C(═O)-alkyl), an aldehyde (e.g., —C(═O)H), or an ester (e.g., —C(═O)—O-alkyl). In some forms, R₂ and R₃ can be independently a methyl, an ethyl, a hexyl, or a hexyl substituted with a hydroxyl.

In some forms, R₂ can be an alkyl, such as an unsubstituted C1-C6 alkyl, a substituted C1-C6 alkyl, an unsubstituted C1-C5 alkyl, an unsubstituted C1-C4 alkyl, an unsubstituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol (e.g., —SH), an alkoxy (e.g., —O-alkyl), a carboxylic acid (—COOH), a ketone (e.g., —C(═O)-alkyl), an aldehyde (e.g., —C(═O)H), or an ester (e.g., —C(═O)—O-alkyl). In some forms, R₂ can be a methyl, an ethyl, a hexyl, or a hexyl substituted with a hydroxyl.

In some forms, the electron withdrawing group R₁ can be a halide, an alkyl halide, a (trifluoromethyl)sulfanyl, a (trifluoromethyl)sulfinyl, a (trifluoromethyl)sulfonyl, a nitro, a nitrile, an aldehyde, a ketone, a carboxyl, a sulfonyl, a sulfinyl, a sulfino, an amido, a sulfonamido, or an aldehyde.

In some forms, the DHA derivative can have any one of the following structures:

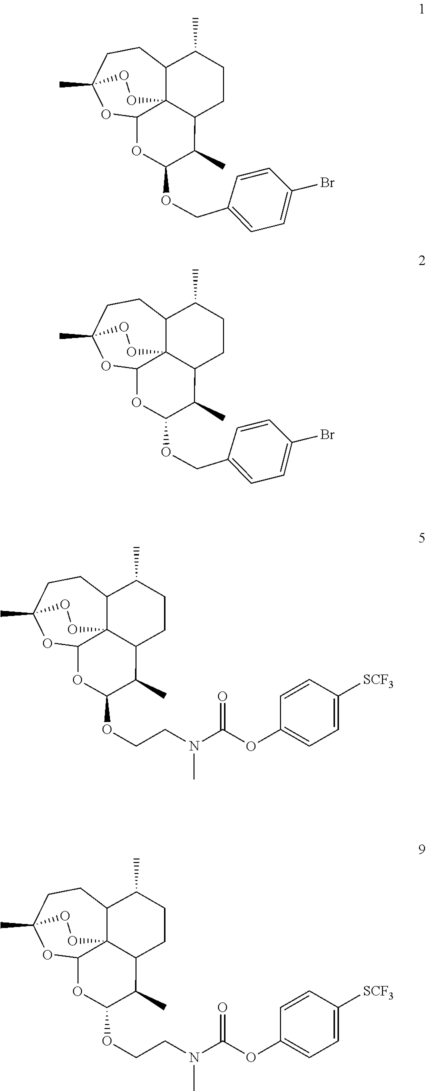

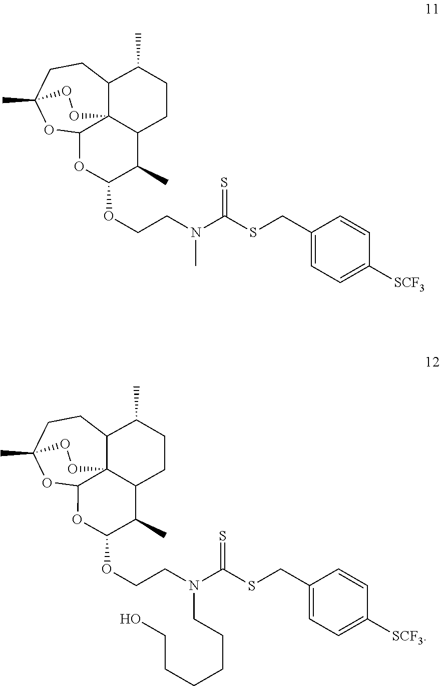

In some forms, the DHA derivative can have any one of the following structures:

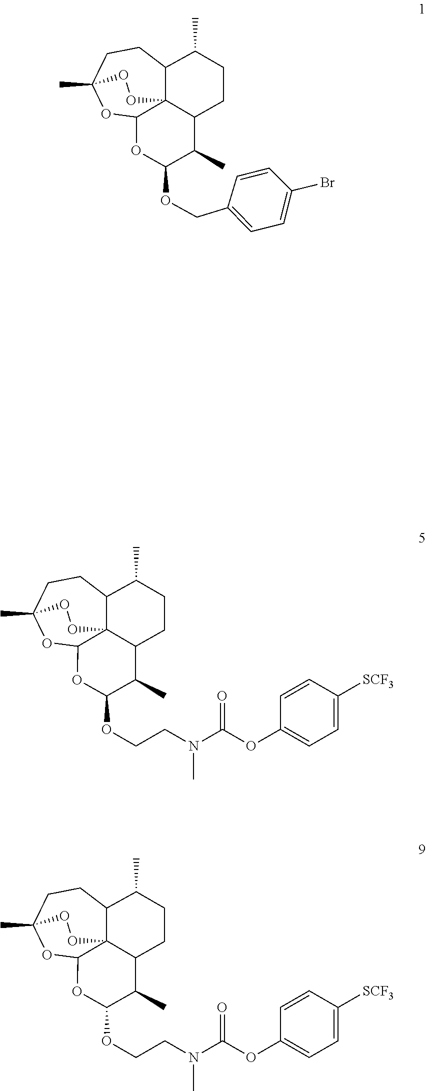

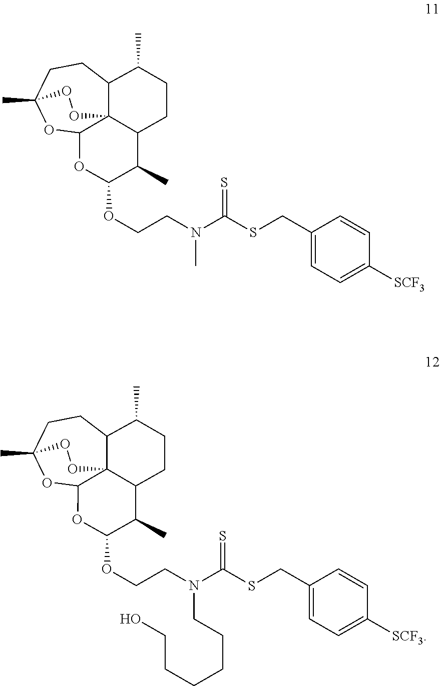

Methods for synthesizing the DHA derivatives are disclosed. The disclosed methods are cost effective and allow for stereospecific (e.g., at C-10 position) synthesis of DHA derivatives.

Methods of using the DHA derivatives or a pharmaceutical composition thereof for treating a cancer in a subject in need thereof are also disclosed. In some forms, the DHA derivatives are particularly useful in treating ovarian cancer and/or colon cancer in a subject in need thereof. In some forms, the DHA derivatives are particularly useful in treating Cisplatin-resistant ovarian cancer in a subject in need thereof.

### BRIEF DESCRIPTION OF THE DRAWINGS

FIGS. 1A-1F are bar graphs showing high-content analysis of total cells (FIGS. 1A-1C) and dead cells (FIGS. 1D-1F) of A2780 (FIGS. 1A and 1D), A2780cisR ovarian cancer (FIGS. 1B and 1E), and HCT-116 colon cancer cells (FIGS. 1C and 1F) treated with DHA and cisplatin (DDP). VC=Vehicle.

FIGS. 2A-2K show in vitro anticancer properties of DHA and its derivatives. FIGS. 2A-2C are bar graphs showing high-content analysis of total cells of NCI-H460 lung cancer treated with DHA (FIG. 2A), 11 (FIG. 2B) and 12 (FIG. 2C). VC=Vehicle. FIGS. 2D-2F are bar graphs showing high-content analysis of dead cells of NCI-H460 lung cancer treated with DHA (FIG. 2D), 11 (FIG. 2E) and 12 (FIG. 2F). VC=Vehicle. FIG. 2G are images showing tumorsphere formation assay showing the number and size of NCI-H460 tumorspheres formed in the presence of DHA (25 μM), 11 (1 μM) and 12 (1 μM) (scale bar, 100 m). FIGS. 211-2J are images showing the wound healing assay with NCI-H460 cells incubated with just the vehicle (FIG. 211), DHA (1 μM) (FIG. 21) and 12 (0.1 μM) (FIG. 2J) (scale bar, 100 m). FIG. 2K is a bar graph comparing cell migration (%) after 24 and 48 hours, between the vehicle, DHA, and 12.

FIG. 3 is a line graph showing in vivo antitumor effects of DHA and 12. The effect of 12 on NCI-H460 xenograft tumors in nude mice with changes in average tumor volume.

### DETAILED DESCRIPTION OF THE INVENTION

### I. Definitions

It is to be understood that the disclosed compounds, compositions, and methods are not limited to specific synthetic methods, specific analytical techniques, or to particular reagents unless otherwise specified, and, as such, may vary. It is also to be understood that the terminology used herein is for the purpose of describing particular forms and embodiments only and is not intended to be limiting.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid. Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, and an amino acid can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

“Alkyl,” as used herein, refers to the radical of saturated aliphatic groups, including straight-chain alkyl groups, branched-chain alkyl, and cycloalkyl (alicyclic). In some forms, a straight chain or branched chain alkyl has 30 or fewer carbon atoms in its backbone (e.g., C₁-C₃₀ for straight chains, C₃-C₃₀ for branched chains), 20 or fewer, 15 or fewer, or 10 or fewer. Alkyl includes methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Likewise, a cycloalkyl is a non-aromatic carbon-based ring composed of at least three carbon atoms, such as a nonaromatic monocyclic or nonaromatic polycyclic ring containing 3-30 carbon atoms, 3-20 carbon atoms, or 3-10 carbon atoms in their ring structure, and have 5, 6 or 7 carbons in the ring structure. Cycloalkyls containing a polycyclic ring system can have two or more non-aromatic rings in which two or more carbons are common to two adjoining rings (i.e., “fused cycloalkyl rings”). Examples of cycloalkyl groups include, but are not limited to, cyclopropyl, cyclobutyl, cyclopentyl, cyclohexyl, cycloheptyl, cyclooctanyl, etc.

“Substituted alkyl” refers to alkyl moieties having one or more substituents replacing a hydrogen on one or more carbons of the hydrocarbon backbone. Such substituents can be any substituents described above, e.g., halogen (such as fluorine, chlorine, bromine, or iodine), hydroxyl, carbonyl (such as a carboxyl, alkoxycarbonyl, formyl, or an acyl), thiocarbonyl (such as a thioester, a thioacetate, or a thioformate), aryl, alkoxyl, aralkyl, phosphonium, phosphanyl, phosphonyl, phosphoryl, phosphate, phosphonate, a phosphinate, amino, amido, amidine, imine, cyano, nitro, azido, oxo, sulfhydryl, thiol, alkylthio, silyl, sulfinyl, sulfate, sulfonate, sulfamoyl, sulfonamido, sulfonyl, heterocyclyl, an aromatic or heteroaromatic moiety. —NRR′, wherein R and R′ are independently hydrogen, alkyl, or aryl, and wherein the nitrogen atom is optionally quaternized; —SR, wherein R is a phosphonyl, a sulfinyl, a silyl a hydrogen, an alkyl, or an aryl; —CN; —NO₂; —COOH; carboxylate; —COR, —COOR, or —CON(R)₂, wherein R is hydrogen, alkyl, or aryl; imino, silyl, ether, haloalkyl (such as —CF3, —CH₂—CF₃, —CCl₃); —CN; —NCOCOCH₂CH₂; —NCOCOCHCH; and —NCS; and combinations thereof.

It will be understood by those skilled in the art that the moieties substituted on the hydrocarbon chain can themselves be substituted, if appropriate. For instance, the substituents of a substituted alkyl may include halogen, hydroxy, nitro, thiols, amino, aralkyl, azido, imino, amido, phosphonium, phosphanyl, phosphoryl (including phosphonate and phosphinate), oxo, sulfonyl (including sulfate, sulfonamido, sulfamoyl and sulfonate), and silyl groups, as well as ethers, alkylthios, carbonyls (including ketones, aldehydes, carboxylates, and esters), haloalkyls, —CN and the like. Cycloalkyls can be substituted in the same manner.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths.

“Heteroalkyl,” as used herein, refers to straight or branched chain, or cyclic carbon-containing alkyl radicals, or combinations thereof, containing at least one heteroatom on the carbon backbone. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “alkenyl” as used herein is a hydrocarbon group of from 2 to 24 carbon atoms and structural formula containing at least one carbon-carbon double bond. Alkenyl groups include straight-chain alkenyl groups, branched-chain alkenyl, and cycloalkenyl. A cycloalkenyl is a non-aromatic carbon-based ring composed of at least three carbon atoms and at least one carbon-carbon double bond, such as a nonaromatic monocyclic or nonaromatic polycyclic ring containing 3-30 carbon atoms and at least one carbon-carbon double bond, 3-20 carbon atoms and at least one carbon-carbon double bond, or 3-10 carbon atoms and at least one carbon-carbon double bond in their ring structure, and have 5, 6 or 7 carbons and at least one carbon-carbon double bond in the ring structure. Cycloalkenyls containing a polycyclic ring system can have two or more non-aromatic rings in which two or more carbons are common to two adjoining rings (i.e., “fused cycloalkenyl rings”) and contain at least one carbon-carbon double bond. Asymmetric structures such as (AB)C=C(C′D) are intended to include both the E and Z isomers. This may be presumed in structural formulae herein wherein an asymmetric alkene is present, or it may be explicitly indicated by the bond symbol C. The term “alkenyl” as used throughout the specification, examples, and claims is intended to include both “unsubstituted alkenyls” and “substituted alkenyls,” the latter of which refers to alkenyl moieties having one or more substituents replacing a hydrogen on one or more carbons of the hydrocarbon backbone. The term “alkenyl” also includes “heteroalkenyl.”

The term “substituted alkenyl” refers to alkenyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, oxo, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkenyl,” as used herein, refers to straight or branched chain, or cyclic carbon-containing alkenyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkenyl group” is a cycloalkenyl group where at least one of the carbon atoms of the ring is substituted with a heteroatom such as, but not limited to, nitrogen, oxygen, sulphur, or phosphorus.

The term “alkynyl group” as used herein is a hydrocarbon group of 2 to 24 carbon atoms and a structural formula containing at least one carbon-carbon triple bond. Alkynyl groups include straight-chain alkynyl groups, branched-chain alkynyl, and cycloalkynyl. A cycloalkynyl is a non-aromatic carbon-based ring composed of at least three carbon atoms and at least one carbon-carbon triple bond, such as a nonaromatic monocyclic or nonaromatic polycyclic ring containing 3-30 carbon atoms and at least one carbon-carbon triple bond, 3-20 carbon atoms and at least one carbon-carbon triple bond, or 3-10 carbon atoms and at least one carbon-carbon triple bond in their ring structure, and have 5, 6 or 7 carbons and at least one carbon-carbon triple bond in the ring structure. Cycloalkynyls containing a polycyclic ring system can have two or more non-aromatic rings in which two or more carbons are common to two adjoining rings (i.e., “fused cycloalkynyl rings”) and contain at least one carbon-carbon triple bond. Asymmetric structures such as (AB)C ≡C(C″D) are intended to include both the E and Z isomers. This may be presumed in structural formulae herein wherein an asymmetric alkyne is present, or it may be explicitly indicated by the bond symbol C. The term “alkynyl” as used throughout the specification, examples, and claims is intended to include both “unsubstituted alkynyls” and “substituted alkynyls,” the latter of which refers to alkynyl moieties having one or more substituents replacing a hydrogen on one or more carbons of the hydrocarbon backbone. The term “alkynyl” also includes “heteroalkynyl.”

The term “substituted alkynyl” refers to alkynyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkynyl,” as used herein, refers to straight or branched chain, or cyclic carbon-containing alkynyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkynyl group” is a cycloalkynyl group where at least one of the carbon atoms of the ring is substituted with a heteroatom such as, but not limited to, nitrogen, oxygen, sulphur, or phosphorus.

“Aryl,” as used herein, refers to C₄-C₂₆-membered aromatic rings or fused ring systems containing one aromatic ring and optionally one or more non-aromatic rings. Examples of aryl groups are benzene, tetralin, indane, etc.

The term “substituted aryl” refers to an aryl group, wherein one or more hydrogen atoms on one or more aromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

“Heterocyclo” and “heterocyclyl” are used interchangeably, and refer to a cyclic radical attached via a ring carbon or nitrogen atom of a monocyclic ring or polycyclic ring system containing 3-30 ring atoms, 3-20 ring atoms, 3-10 ring atoms, or 5-6 ring atoms, where the polycyclic ring system contains one or more non-aromatic rings and optionally one or more aromatic rings, where at least one non-aromatic ring contains carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Heterocycles can be a heterocycloalkyl, a heterocycloalkenyl, a heterocycloalkynyl, etc., such as piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₃-C₂₆-membered aromatic rings or fused ring systems containing one aromatic ring and optionally one or more non-aromatic rings, in which one or more carbon atoms on the aromatic ring structure have been substituted with a heteroatom. Suitable heteroatoms include, but are not limited to, oxygen, sulfur, and nitrogen. Examples of heteroaryl groups pyrrole, furan, thiophene, imidazole, oxazole, thiazole, triazole, tetrazole, pyrazole, pyridine, pyrazine, pyridazine and pyrimidine, and the like. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl.”

The term “substituted heteroaryl” refers to a heteroaryl group in which one or more hydrogen atoms on one or more heteroaromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

The term “polyaryl” refers to a fused ring system that includes two or more aromatic rings and optionally one or more non-aromatic rings. Examples of polyaryl groups are naphthalene, anthracene, phenanthrene, chrysene, pyrene, corannulene, coronene, etc. When a fused ring system containing two or more aromatic rings and optionally one or more non-aromatic rings, in which one or more carbon atoms on two or more aromatic ring structures have been substituted with a heteroatom, the fused ring system can be referred to as a “polyheteroaryl”. When a fused ring system containing two or more aromatic rings and optionally one or more non-aromatic rings, in which one or more carbon atoms in the fused ring system is substituted with a heteroatom it can be referred to as a “heteropolyaryl.”

The term “substituted polyaryl” refers to a polyaryl in which one or more of the aryls are substituted, with one or more substituents including, but not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof. When a polyheteroaryl is involved, the chemical moiety can be referred to as a “substituted polyheteroaryl.”

The term “cyclic ring” or “cyclic group” refers to a substituted or unsubstituted monocyclic ring or a substituted or unsubstituted polycyclic ring (such as those formed from single or fused ring systems), such as a substituted or unsubstituted cycloalkyl, a substituted or unsubstituted cycloalkenyl, a substituted or unsubstituted cycloalkynyl, or a substituted or unsubstituted heterocyclyl, that have from three to 30 carbon atoms, as geometric constraints permit. The substituted cycloalkyls, cycloalkenyls, cycloalkynyls, and heterocyclyls are substituted as defined above for the alkyls, alkenyls, alkynyls, and heterocyclyls, respectively.

The term “aralkyl” as used herein is an aryl group or a heteroaryl group having an alkyl, alkynyl, or alkenyl group as defined above attached to the aromatic group, such as an aryl, a heteroaryl, a polyaryl, or a polyheteroaryl. An example of an aralkyl group is a benzyl group.

The terms “alkoxyl” or “alkoxy,” “aroxy” or “aryloxy,” generally describe compounds represented by the formula —OR^{v}, wherein R^{v} includes, but is not limited to, a substituted or unsubstituted alkyl, a substituted or unsubstituted alkenyl, a substituted or unsubstituted alkynyl, a substituted or unsubstituted cycloalkyl, a substituted or unsubstituted heterocyclyl, a substituted or unsubstituted cycloalkenyl, a substituted or unsubstituted heterocycloalkenyl, a substituted or unsubstituted aryl, a substituted or unsubstituted heteroaryl, a substituted or unsubstituted polyaryl, a substituted or unsubstituted polyheteroaryl, a substituted or unsubstituted arylalkyl, a substituted or unsubstituted heteroalkyl, a substituted or unsubstituted alkylaryl, a substituted or unsubstituted alkylheteroaryl, a substituted or unsubstituted aralkyl, a substituted or unsubstituted carbonyl, a phosphonium, a phosphanyl, a phosphonyl, a sulfinyl, a silyl, a thiol, an amido, and an amino. Exemplary alkoxyl groups include methoxy, ethoxy, propyloxy, tert-butoxy and the like. A “lower alkoxy” group is an alkoxy group containing from one to six carbon atoms. An “ether” is two functional groups covalently linked by an oxygen as defined below. Accordingly, the substituent of an alkyl that renders that alkyl an ether is or resembles an alkoxyl, such as can be represented by one of —O-alkyl, —O-alkenyl, —O-alkynyl, —O-arakyl, —O-aryl, —O-heteroaryl, —O-polyaryl, —O-polyheteroaryl, —O-heterocyclyl, etc.

The term “substituted alkoxy” refers to an alkoxy group having one or more substituents replacing one or more hydrogen atoms on one or more carbons of the alkoxy backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, oxo, sulfate, sulfonate, sulfamoyl, sulfonamido, sulfonyl, heterocyclyl, alkylaryl, haloalkyl, —CN, aryl, heteroaryl, and combinations thereof.

The term “ether” as used herein is represented by the formula A²OA¹, where A² and A¹ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a phosphonyl, a sulfinyl, a silyl, a thiol, a substituted or unsubstituted carbonyl, an alkoxy, an amido, or an amino, described above.

The term “polyether” as used herein is represented by the formula:

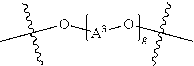

where A³ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a substituted or unsubstituted carbonyl, an alkoxy, an amido, or an amino, described above; g can be a positive integer from 1 to 30.

The term “phenoxy” is art recognized and refers to a compound of the formula —OR^{v} wherein R^{v} is C₆H₅ (i.e., —O—C₆H₅). One of skill in the art recognizes that a phenoxy is a species of the aroxy genus.

The term “substituted phenoxy” refers to a phenoxy group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The terms “aroxy” and “aryloxy,” as used interchangeably herein, are represented by —O-aryl or —O-heteroaryl, wherein aryl and heteroaryl are as defined herein.

The terms “substituted aroxy” and “substituted aryloxy,” as used interchangeably herein, represent —O-aryl or —O-heteroaryl, having one or more substituents replacing one or more hydrogen atoms on one or more ring atoms of the aryl and heteroaryl, as defined herein. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “amino” as used herein includes the group

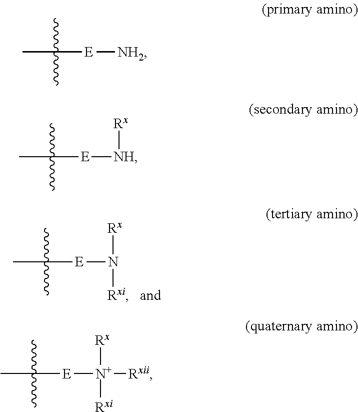

wherein, E is absent, or E is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl, wherein independently of E, R^{x}, R^{xi}, and R^{xii} each independently represent a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. The term “quaternary amino” also includes the groups where the nitrogen, R^{x}, R^{xi}, and R^{xii} with the N⁺ to which they are attached complete a heterocyclyl or heteroaryl having from 3 to 14 atoms in the ring structure. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The terms “amide” or “amido” are used interchangeably, refer to both “unsubstituted amido” and “substituted amido” and are represented by the general formula:

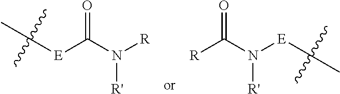

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted aryl, substituted or unsubstituted heteroaryl, a substituted or unsubstituted polyaryl, a substituted or unsubstituted polyheteroaryl, or a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. In some forms, when E is oxygen, a carbamate is formed. It is understood by those of ordinary skill in the art, that the E groups listed above are divalent (e.g., methylene, ethane-1,2-diyl, ethene-1,2-diyl, 1,4-phenylene, cyclohexane-1,2-diyl).

“Carbonyl,” as used herein, is art-recognized and includes such moieties as can be represented by the general formula:

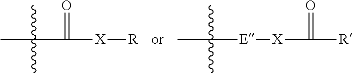

wherein X is a bond, or represents an oxygen or a sulfur, and R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″, or a pharmaceutical acceptable salt; E″ is absent, or E″ is a substituted or unsubstituted alkyl, a substituted or unsubstituted alkenyl, a substituted or unsubstituted alkynyl, a substituted or unsubstituted aralkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl; R′ represents a hydrogen, a substituted or unsubstituted alkyl, a substituted or unsubstituted alkenyl, a substituted or unsubstituted alkynyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″; R″ represents a hydroxyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof. It is understood by those of ordinary skill in the art, that the E″ groups listed above are divalent (e.g., methylene, ethane-1,2-diyl, ethene-1,2-diyl, 1,4-phenylene, cyclohexane-1,2-diyl). Where X is oxygen and R is defined as above, the moiety is also referred to as a carboxyl group. When X is oxygen and R is hydrogen, the formula represents a “carboxylic acid.” Where X is oxygen and R′ is hydrogen, the formula represents a “formate.” Where X is oxygen and R or R′ is not hydrogen, the formula represents an “ester.” In general, where the oxygen atom of the above formula is replaced by a sulfur atom, the formula represents a “thiocarbonyl” group. Where X is sulfur and R or R′ is not hydrogen, the formula represents a “thioester.” Where X is sulfur and R is hydrogen, the formula represents a “thiocarboxylic acid.” Where X is sulfur and R′ is hydrogen, the formula represents a “thioformate.” Where X is a bond and R is not hydrogen, the above formula represents a “ketone.” Where X is a bond and R is hydrogen, the above formula represents an “aldehyde.”

The term “phosphanyl” is represented by the formula

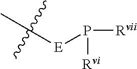

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi} and R^{vii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphonium” is represented by the formula

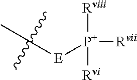

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi}, R^{vii}, and R^{viii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi}, R^{vii}, and R^{viii} taken together with the P⁺ atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphonyl” is represented by the formula

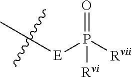

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, oxygen, alkoxy, aroxy, or substituted alkoxy or substituted aroxy, wherein, independently of E, R^{vi} and R^{vii} are independently a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphoryl” defines a phosphonyl in which E is absent, oxygen, alkoxy, aroxy, substituted alkoxy or substituted aroxy, as defined above, and independently of E, R^{vi} and R^{vii} are independently hydroxyl, alkoxy, aroxy, substituted alkoxy or substituted aroxy, as defined above. When E is oxygen, the phosphoryl cannot be attached to another chemical species, such as to form an oxygen-oxygen bond, or other unstable bonds, as understood by one of ordinary skill in the art. When E, R^{vi} and R^{vii} are substituted, the substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfinyl” is represented by the formula

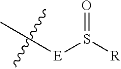

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a silyl, a thiol, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonyl” is represented by the formula

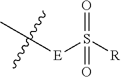

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonic acid” refers to a sulfonyl, as defined above, wherein R is hydroxyl, and E is absent, or E is substituted or unsubstituted cycloalkyl, substituted or unsubstituted heterocyclyl, substituted or unsubstituted alkylaryl, substituted or unsubstituted arylalkyl, substituted or unsubstituted aryl, a substituted or unsubstituted polyaryl, a substituted or unsubstituted polyheteroaryl, or substituted or unsubstituted heteroaryl. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfate” refers to a sulfonyl, as defined above, wherein E is absent, oxygen, alkoxy, aroxy, substituted alkoxy or substituted aroxy, as defined above, and R is independently hydroxyl, alkoxy, aroxy, substituted alkoxy or substituted aroxy, as defined above. When E is oxygen, the sulfate cannot be attached to another chemical species, such as to form an oxygen-oxygen bond, or other unstable bonds, as understood by one of ordinary skill in the art. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonate” refers to a sulfonyl, as defined above, wherein E is oxygen, alkoxy, aroxy, substituted alkoxy or substituted aroxy, as defined above, and R is independently hydrogen, substituted or unsubstituted alkyl, substituted or unsubstituted alkenyl, substituted or unsubstituted alkynyl, substituted or unsubstituted amino, substituted or unsubstituted cycloalkyl, substituted or unsubstituted heterocyclyl, substituted or unsubstituted aralkyl, substituted or unsubstituted alkylaryl, substituted or unsubstituted arylalkyl, substituted or unsubstituted aryl, substituted or unsubstituted heteroaryl, a substituted or unsubstituted polyaryl, a substituted or unsubstituted polyheteroaryl, —(CH₂)ₘ—R′″, R′″ represents a hydroxy group, substituted or unsubstituted carbonyl group, an aryl, a cycloalkyl ring, a cycloalkenyl ring, a heterocycle, an amido, an amino, or a polycycle; and m is zero or an integer ranging from 1 to 8. When E is oxygen, sulfonate cannot be attached to another chemical species, such as to form an oxygen-oxygen bond, or other unstable bonds, as understood by one of ordinary skill in the art. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfamoyl” refers to a sulfonamide or sulfonamide represented by the formula

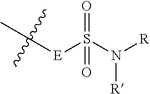

wherein E is absent, or E is substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted cycloalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “silyl group” as used herein is represented by the formula —SiRR′R,″ where R, R′, and R″ can be, independently, a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, a phosphonyl, a sulfinyl, a thiol, an amido, an amino, an alkoxy, or an oxo, described above. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The terms “thiol” are used interchangeably and are represented by —SR, where R can be a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, an amido, an amino, an alkoxy, an oxo, a phosphonyl, a sulfinyl, or a silyl, described above. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted, independently, with the substituents described above in the definition of “substituted.”

The numerical ranges disclose individually each possible number that such a range could reasonably encompass, as well as any sub-ranges and combinations of sub-ranges encompassed therein. For example, in a given range carbon range of C₃-C₉, the range also discloses C₃, C₄, C₅, C₆, C₇, C₈, and C₉, as well as any subrange between these numbers (for example, C₄-C₆), and any possible combination of ranges possible between these values. In yet another example, a given temperature range may be from about 25° C. to 30° C., where the range also discloses temperatures that can be selected independently from about 25, 26, 27, 28, 29, and 30° C., as well as any range between these numbers (for example, 26 to 28° C.), and any possible combination of ranges between these values.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted with, independently, with the substituents described above in the definition of “substituted.”

“oxo” refers to ═O.

The compounds and substituents can be substituted, independently, with the substituents described above in the definition of “substituted.”

Numerical ranges such as ranges of C₁-C₃₀, C₄-C₃₀, C₃-C₃₀, C₁-C₂₀, C₄-C₂₀, C₃-C₂₀, C₁-C₁₀, C₄-C₁₀, C₃-C₁₀, C₁-C₆, C₄-C₆, C₃-C₆, C₁-C₄, C₃-C₄, C₁-C₉, C₁-C₈, C₁-C₇, C₁-C₅, C₁-C₃, C₁-C₂, C₃-C₉, C₃-C₉, C₃-C₈, C₃-C₇, C₃-C₅, C₃-C₄, C₄-C₂₅, C₄-C₂₀, C₄-C₁₈, C₄-C₁₆, C₄-C₁₅, C₄-C₁₄, C₄-C₁₃, C₄-C₁₂, C₄-C₉, C₄-C₈, C₄-C₇, C₄-C₅, etc. The ranges disclose individually each possible number that such a range could reasonably encompass, as well as any sub-ranges and combinations of sub-ranges encompassed therein. For example, in a given range carbon range of C₃-C₉, the range also discloses C₃, C₄, C₅, C₆, C₇, C₈, and C₉, as well as any subrange between these numbers (for example, C₄-C₆), and any possible combination of ranges possible between these values. In yet another example, a given temperature range may be from about 25° C. to 30° C., where the range also discloses temperatures that can be selected independently from about 25, 26, 27, 28, 29, and 30° C., as well as any range between these numbers (for example, 26 to 28° C.), and any possible combination of ranges between these values.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

### II. Compositions

DHA derivatives have been developed. The DHA derivatives disclosed herein contain a stereospecific C-10 ether linkage and an aryl pharmacophore moiety.

In some forms, the DHA derivatives possess anticancer properties that are comparable to or better than the parent DHA and/or an existing anticancer drug (e.g., Cisplatin, also referred to as “DDP”), and are useful in anticancer treatments (such as ovarian cancer and colon cancer).

For example, the DHA derivatives show cytotoxicity against cancer cells (e.g., as indicated by its IC₅₀ values), such as ovarian cancer cells (e.g., A2780 cells and/or A2780CisR cells) and/or colon cancer cells (e.g., HCT-116 cells), comparable to (e.g., concentration in μM on the same order of magnitude) or better than (e.g., concentration in μM that is at least 1 order of magnitude lower) that of parent DHA and/or Cisplatin.

In some forms, the DHA derivatives show a cytotoxicity against A2780 cells that is similar to a cytotoxicity against A2780CisR cells, and thereby overcoming Cisplatin resistance in ovarian cancer cells.

Pharmaceutical formulations containing the DHA derivatives are also disclosed.

### A. Dihydroartemisinin Derivatives

In some forms, the DHA derivatives can have the structure of Formula Ia or Ib:

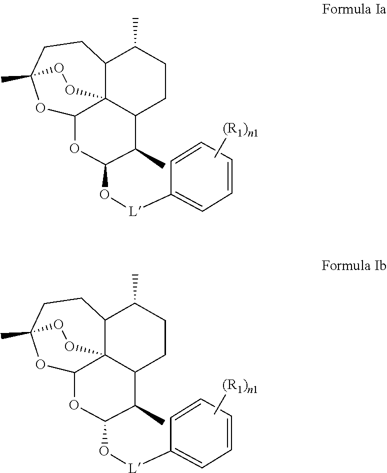

wherein: (i) R₁ can be an electron withdrawing group; (ii) n1 can be an integer from 0 to 5, from 1 to 5, from 1 to 4, from 1 to 3, or 1 or 2, such as 1; and (iii) L′ can be

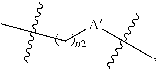

(a) n2 can be an integer from 1 to 6, from 1 to 5, from 1 to 4, from 1 to 3, or 1 or 2; (b) A′ can be a bond or

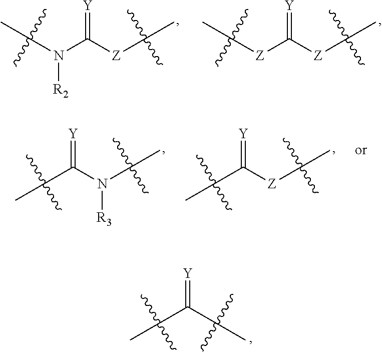

each occurrence of Y and Z are independently O or S; and (c) R₂ and R₃ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

In some forms, A′ can be a bond or

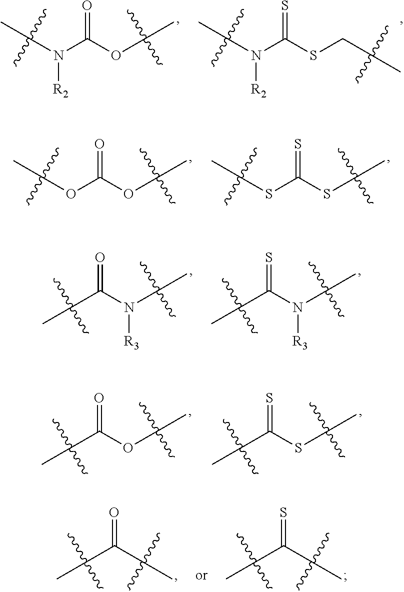

and R₂ and R₃ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

It is understood that the linker L′ can attach to the oxygen at C-10 position via either side. When A′ is

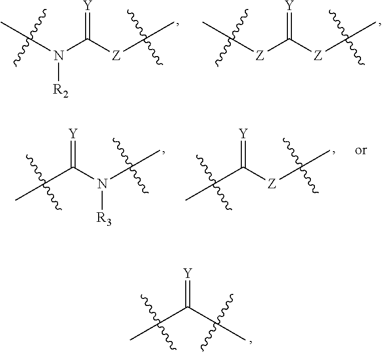

each occurrence of Y and Z are independently O or S (such as

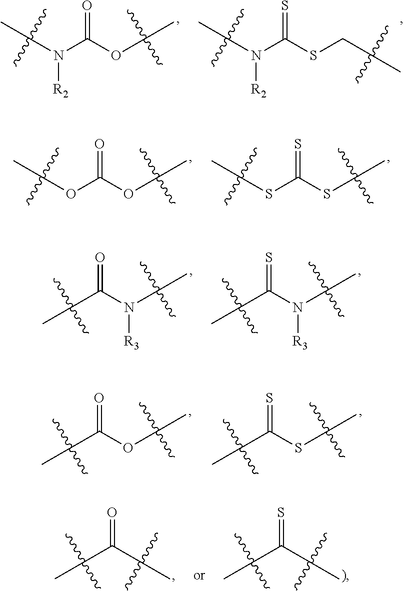

it is understood that either side of the moiety can attach to the phenyl group. For example, when A′ is

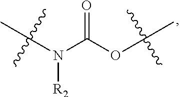

L′ can be either

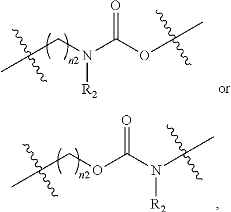

and L′ can attach to the C-10 oxygen atom via the alkylene or via the oxygen or nitrogen atom at the terminus of the linker.

In some forms, the DHA derivatives can have the structure of Formula IIa or IIb:

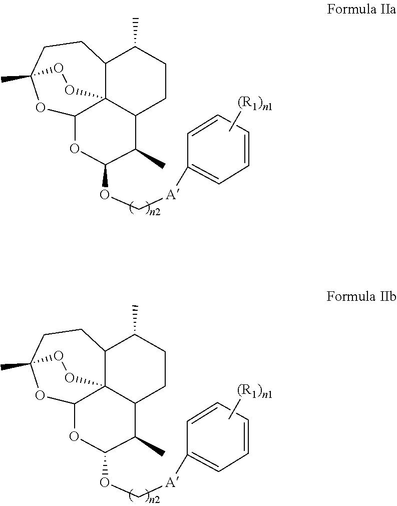

wherein: (i) R₁ can be an electron withdrawing group; (ii) n1 can be an integer from 0 to 5, from 1 to 5, from 1 to 4, from 1 to 3, or 1 or 2, such as 1; (iii) n2 can be an integer from 1 to 6, from 1 to 5, from 1 to 4, from 1 to 3, or 1 or 2; (iv) A′ can be a bond,

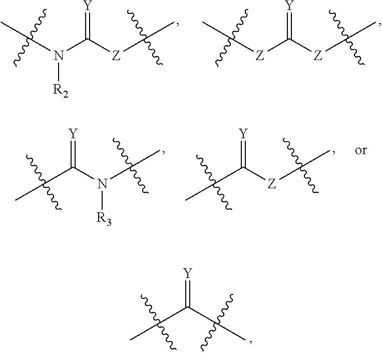

each occurrence of Y and Z are independently O or S (such as

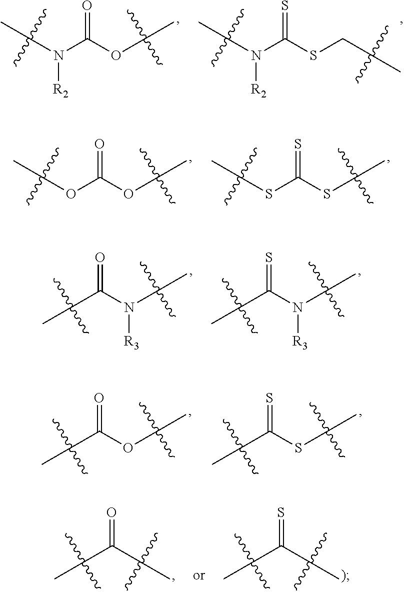

wherein R₂ and R₃ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

In some forms, the DHA derivatives can have the structure of Formula IIa. In some forms, A′ can be a bond,

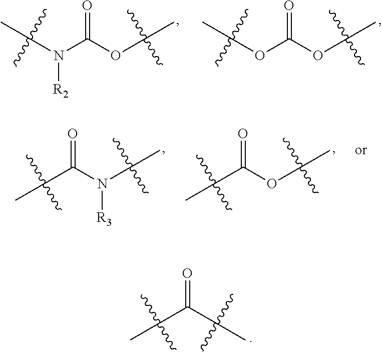

In some forms, A′ can be a bond,

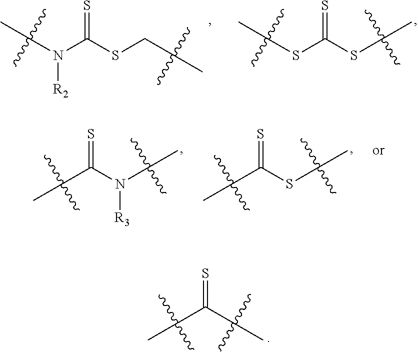

In some forms, A′ can be a bond,

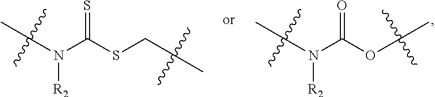

wherein R₂ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

In some forms, A′ can be a bond.

In some forms, the DHA derivatives can have the structure of Formula IIIa, IIIb or IIIc:

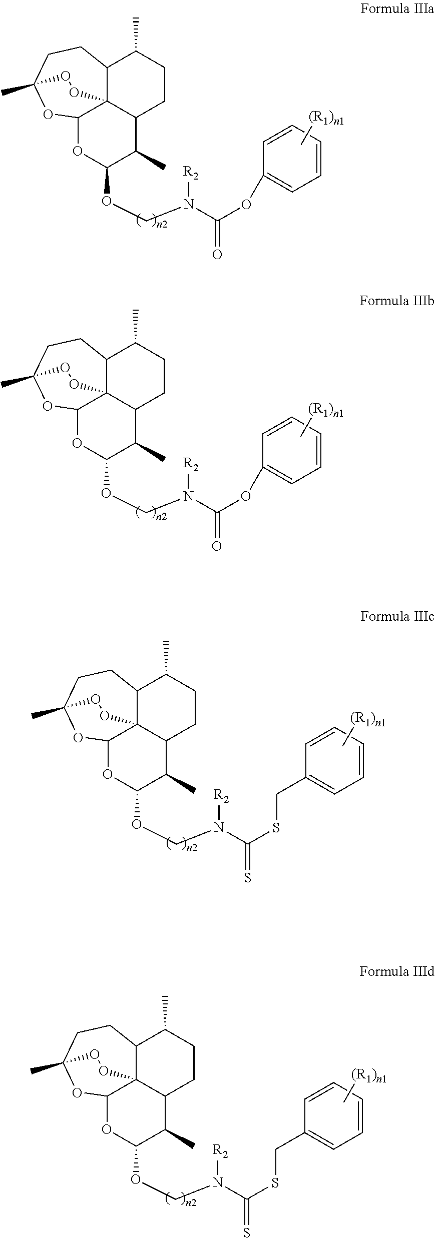

wherein: (i) R₁ can be an electron withdrawing group; (ii) n1 can be an integer from 0 to 5, from 1 to 5, from 1 to 4, from 1 to 3, or 1 or 2, such as 1; (iii) n2 can be an integer from 1 to 6, from 1 to 5, from 1 to 4, from 1 to 3, or 1 or 2; and (iv) R₂ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester). In some forms, for any forms of the compounds disclosed herein, n1 can be an integer from 1 to 3, or 1 or 2, such as 1. In some forms, n1 can be 1 or 2. In some forms, n2 can be an integer from 1 to 3, or 1 or 2. In some forms, n2 can be 1 or 2.

In some forms, for any forms of the compounds disclosed herein, the electron withdrawing group R₁ can be a halide, an alkyl halide, a (trifluoromethyl)sulfanyl, a (trifluoromethyl)sulfinyl, a (trifluoromethyl)sulfonyl, a nitro, a nitrile, an aldehyde, a ketone, a carboxyl, a sulfonyl, a sulfinyl, a sulfino, an amido, a sulfonamido, or an aldehyde.

In some forms, for any forms of the compounds disclosed herein, the electron withdrawing group R₁ can be a halide (e.g., fluoride, chloride, bromide, such as bromide) or a (trifluoromethyl)sulfanyl.

For any forms of the compounds disclosed herein, when R₂ and/or R₃ are/is an alkyl, the alkyl can be a substituted alkyl or an unsubstituted alkyl. In some forms, the alkyl can be an unsubstituted alkyl. In some forms, the alkyl can be a substituted alkyl, where the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl (e.g. benzyl), a carbonyl (e.g. carboxyl), an alkoxy (e.g. methoxy, ethoxy, aryloxy, benzoether, etc.), a halide, a hydroxyl, or a haloalkyl, or a combination thereof.

In some forms, the alkyl can be a substituted alkyl, where the substituents can be independently an unsubstituted alkyl, an unsubstituted alkenyl, an unsubstituted alkynyl, an unsubstituted heterocyclyl, an unsubstituted aryl, an unsubstituted heteroaryl, an unsubstituted polyaryl, an unsubstituted polyheteroaryl, an unsubstituted aralkyl (e.g. benzyl), an alkoxy (e.g. methoxy, ethoxy, aryloxy, benzoether, etc.), a carbonyl (e.g. carboxyl), a halide, a hydroxyl, or a haloalkyl, or a combination thereof. In some forms, the alkyl can be a substituted alkyl, where the substituents can be an unsubstituted alkyl (e.g. methyl, ethyl, propyl, butyl, pentyl, hexyl, etc.), an alkoxy (e.g. methoxy, ethoxy, etc.), a hydroxyl, or a combination thereof.

In some forms, the alkyl can be a substituted alkyl, where the substituents can be independently a hydroxyl, a thiol (e.g., —SH), an alkoxy (e.g., —O-alkyl), a carboxylic acid (—COOH), a ketone (e.g., —C(═O)-alkyl), an aldehyde (e.g., —C(═O)H), or an ester (e.g., —C(═O)—O-alkyl).

For any forms of the compounds disclosed herein, when R₂ and/or R₃ are/is an alkyl, the alkyl can be a linear alkyl, a branched alkyl, or a cyclic alkyl (either monocyclic or polycyclic). The terms “cyclic alkyl” and “cycloalkyl” are used interchangeably herein. Exemplary alkyl include a linear C1-C₃₀ alkyl, a branched C₄-C₃₀ alkyl, a cyclic C₃-C₃₀ alkyl, a linear C₁-C₂₀ alkyl, a branched C₄-C₂₀ alkyl, a cyclic C₃-C₂₀ alkyl, a linear C₁-C₁₀ alkyl, a branched C₄-C₁₀ alkyl, a cyclic C₃-C₁₀ alkyl, a linear C₁-C₆ alkyl, a branched C₄-C₆ alkyl, a cyclic C₃-C₆ alkyl, a linear C₁-C₄ alkyl, cyclic C₃-C₄ alkyl, such as a linear C₁-C₁₀, C₁-C₉, C₁-C₈, C₁-C₇, C₁-C₆, C₁-C₅, C₁-C₄, C₁-C₃, or C₁-C₂ alkyl group, a branched C₃-C₉, C₃-C₉, C₃-C₈, C₃-C₇, C₃-C₆, C₃-C₅, or C₃-C₄ alkyl group, or a cyclic C₃-C₉, C₃-C₉, C₃-C₈, C₃-C₇, C₃-C₆, C₃-C₅, or C₃-C₄ alkyl group. The cyclic alkyl can be a monocyclic or polycyclic alkyl, such as a C₄-C₃₀, C₄-C₂₅, C₄-C₂₀, C₄-C₁₈, C₄-C₁₆, C₄-C₁₅, C₄-C₁₄, C4-C13, C₄-C₁₂, C₄-C₁₀, C₄-C₉, C₄-C₈, C₄-C₇, C₄-C₆, or C₄-C₅ monocyclic or polycyclic alkyl group.

For example, for any forms of the compounds disclosed herein, when R₂ and/or R₃ are/is an alkyl, such as an unsubstituted C1-C6 alkyl, a substituted C1-C6 alkyl, an unsubstituted C1-C5 alkyl, an unsubstituted C1-C4 alkyl, an unsubstituted C1-C3 alkyl, for example, a methyl, an ethyl, an n-propyl, an isopropyl, an n-butyl, an iso-butyl, a tert-butyl, a cyclobutyl, an n-pentyl, an iso-pentyl, a tert-pentyl, a cyclopentyl, an n-hexyl, an iso-hexyl, a tert-hexyl, or a cyclohexyl, each optionally substituted with a hydroxyl, a thiol (e.g., —SH), an alkoxy (e.g., —O-alkyl), a carboxylic acid (—COOH), a ketone (e.g., —C(═O)-alkyl), an aldehyde (e.g., —C(═O)H), or an ester (e.g., —C(═O)—O-alkyl).

In some forms, for any forms of the compounds disclosed herein, R₂ and/or R₃ can be independently a methyl, an ethyl, a hexyl, or a hexyl substituted with a hydroxyl group.

In some forms, the DHA derivative can have any one of the following structures:

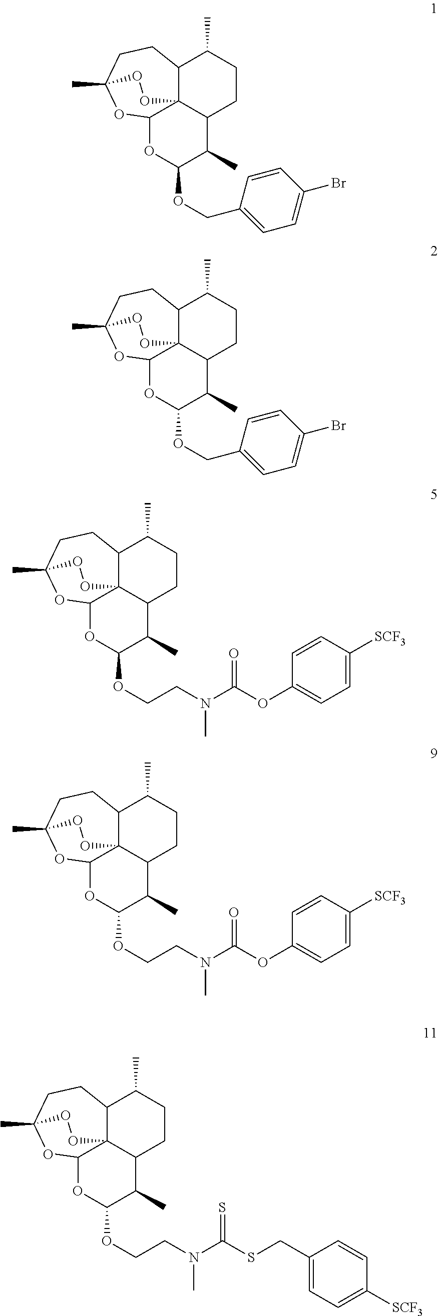

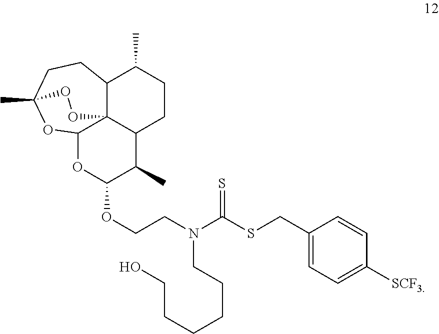

In some forms, the DHA derivative can have any one of the following structures:

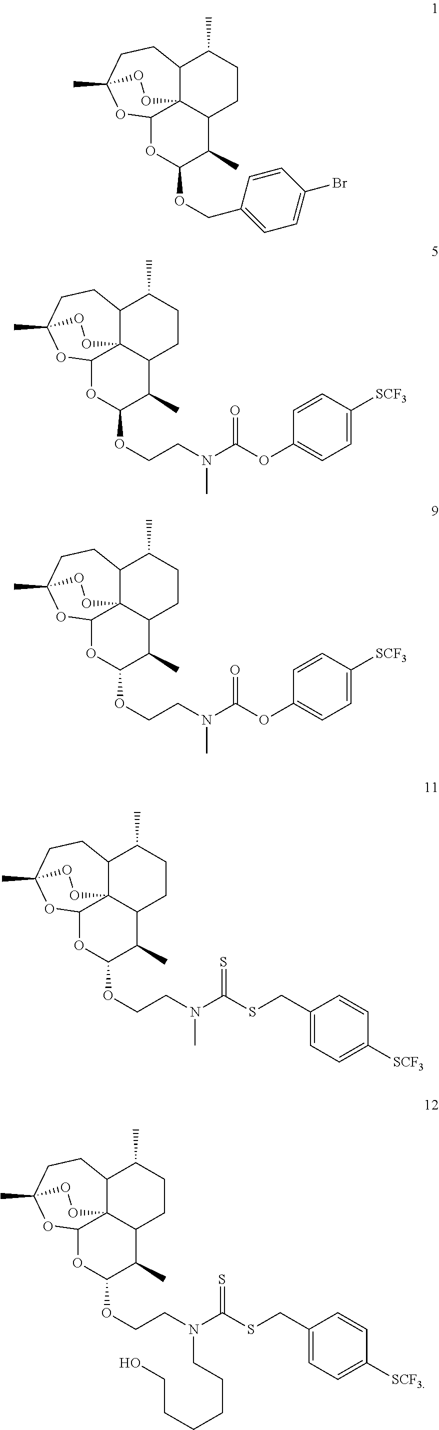

The compounds may be neutral or may be one or more pharmaceutically acceptable salts, crystalline forms, non-crystalline forms, hydrates, or solvates, or a combination thereof. References to the compounds may refer to the neutral molecule, and/or those additional forms thereof collectively and individually from the context. Pharmaceutically acceptable salts of the compounds include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

### B. Pharmaceutical Formulations

Pharmaceutical formulations that contain one or more the DHA derivative(s) disclosed herein, in a form suitable for administration to a mammal, are disclosed. Typically, the compound(s) in the pharmaceutical formulation is present in an amount effective to ameliorate one or more symptoms associated with a cancer in a subject.

The pharmaceutical formulation containing the compound(s) may also include one more pharmaceutically acceptable carriers and/or one or more pharmaceutically acceptable excipients. For example, the pharmaceutical formulation may be in the form of a liquid, such as a solution or a suspension, and contain one or more the disclosed compounds in an aqueous medium and, optionally, one or more suitable excipients for the liquid formulation. Optionally, the pharmaceutical formulation is in a solid form, and contains one or more the disclosed compounds and one or more suitable excipients for a solid formulation.

### 1. Carriers and Excipients

The pharmaceutical formulation can contain one or more pharmaceutically acceptable carriers and/or one or more pharmaceutically acceptable excipients. Suitable pharmaceutically acceptable carriers and excipients are generally recognized as safe (GRAS), and may be administered to an individual without causing undesirable biological side effects or unwanted interactions.

Representative carriers and excipients that can be used in the pharmaceutical formulations include solvents (including buffers), diluents, pH modifying agents, preservatives, antioxidants, suspending agents, wetting agents, viscosity modifiers, tonicity agents, and stabilizing agents, and a combination thereof.

In some forms, the compounds can be dissolved or suspended in a suitable carrier to form a liquid pharmaceutical formulation, such as sterile saline, phosphate buffered saline (PBS), balanced salt solution (BSS), viscous gel, or other pharmaceutically acceptable carriers for administration. The pharmaceutical formulation may also be a sterile solution, suspension, or emulsion in a nontoxic, parenterally acceptable diluent or solvent.

Excipients can be added to a liquid or solid pharmaceutical formulation to assist in sterility, stability (e.g. shelf-life), integration, and to adjust and/or maintain pH or isotonicity of the compounds in the pharmaceutical formulation, such as diluents, pH modifying agents, preservatives, antioxidants, suspending agents, wetting agents, viscosity modifiers, tonicity agents, and stabilizing agents, and a combination thereof.

### 2. Form

The pharmaceutical formulation containing one or more the disclosed DHA derivative(s) can be in a liquid form or a solid form, such as a liquid formulation or a solid formulation for oral administration, mucosal administration, or parenteral administration (e.g. intramuscular administration, intravenous administration, intraperitoneal administration, and subcutaneous administration), or topical administration, to a subject.

a. Oral Formulations

In some forms, the pharmaceutical formulation containing one or more the disclosed compound(s) can be in a form suitable for oral administration to a subject, such as a mammal (i.e. an oral formulation). Oral administration may involve swallowing, so that the compound(s) enter the gastrointestinal tract, or buccal or sublingual administration may be employed by which the compound(s) enter(s) the blood stream directly from the mouth.

Formulations suitable for oral administration include solid formulations such as tablets, capsules containing particulates, liquids, powders, lozenges (including liquid-filled lozenges), chews, multi- and nano-particulates, gels, solid solutions, liposomes, films, ovules, sprays, and liquid formulations.

Liquid formulations for oral administration include suspensions, solutions, syrups, and elixirs. Such oral formulations may be employed as fillers in soft or hard capsules and can contain one or more suitable carriers and/or excipients, for example, water, ethanol, polyethylene glycol, propylene glycol, chitosan polymers and chitosan derivatives (e.g. N-trimethylene chloride chitosan, chitosan esters, chitosan modified with hydrophilic groups, such as amino groups, carboxyl groups, sulfate groups, etc.), methylcellulose, a suitable oil, one or more emulsifying agents, and/or suspending agents. Liquid formulations for oral administration may also be prepared by the reconstitution of a solid, for example, from a sachet.

Optionally, the compound(s) is/are included in a fast-dissolving and/or fast-disintegrating dosage form.

For tablet or capsule dosage forms, in addition to the compound(s) described herein, tablets generally contain disintegrants, binders, diluents, surface active agents, lubricants, glidants, antioxidants, colourants, flavouring agents, preservatives, or taste masking agents, or a combination thereof.

Examples of suitable disintegrants for forming a table or capsule dosage form containing the compound(s) include, but are not limited to, sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant can have a concentration in a range from about 1 wt % to about 25 wt %, from about 5 wt % to about 20 wt % of the tablet or capsule dosage form containing the compound(s).

Binders are generally used to impart cohesive qualities to a tablet formulation. Suitable binders for forming a tablet or capsule formulation containing the compound(s) include, but are not limited to, microcrystalline cellulose, gelatin, sugars, polyethylene glycol, natural and synthetic gums, polyvinylpyrrolidone, pregelatinised starch, chitosan polymers and chitosan derivatives (e.g. N-trimethylene chloride chitosan, chitosan esters, chitosan modified with hydrophilic groups, such as amino groups, carboxyl groups, sulfate groups, etc.), hydroxypropyl cellulose, and hydroxypropyl methylcellulose.

Suitable diluents for forming a table or capsule formulation containing the compound(s) include, but are not limited to, lactose (as, for example, the monohydrate, spray-dried monohydrate or anhydrous form), chitosan polymers and chitosan derivatives (e.g. N-trimethylene chloride chitosan, chitosan esters, chitosan modified with hydrophilic groups, such as amino groups, carboxyl groups, sulfate groups, etc.), N-sulfonated derivatives of chitosan, quaternarized derivatives of chitosan, carbosyalkylated chitosan, microcrystalline chitosan, mannitol, xylitol, dextrose, sucrose, sorbitol, microcrystalline cellulose, starch and dibasic calcium phosphate dihydrate.

Tablet or capsule formulations containing the compound(s) may also contain surface active agents, such as sodium lauryl sulfate and polysorbate 80, and glidants such as silicon dioxide and talc. When present, surface active agents can have a concentration in a range from about 0.2 wt % to 5 wt % of the tablet or capsule formulation.

Tablet or capsule formulations containing the compound(s) also can contain lubricants, such as magnesium stearate, calcium stearate, zinc stearate, sodium stearyl fumarate, and mixtures of magnesium stearate with sodium lauryl sulphate. Lubricants can have a concentration in a range from about 0.25 wt % to 10 wt %, from about 0.5 wt % to about 3 wt % of the tablet or capsule formulation.

Other possible excipients included in a tablet or capsule formulation containing the compound(s) include glidants (e.g. Talc or colloidal anhydrous silica at about 0.1 wt % to about 3 wt % of the table or capsule formulation), antioxidants, colourants, flavouring agents, preservatives and taste-masking agents. When present, glidants can have a concentration in a range from about 0.2 wt % to 1 wt % of the tablet or capsule formulation.

An exemplary tablet formulation contains up to about 80 wt % of the compound(s) described herein, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet or capsule blends, including the compound(s) and one or more suitable excipients, may be compressed directly or by roller to form tablets. Tablet or capsule blends or portions of the blends may alternatively be wet-, dry-, or melt-granulated, melt congealed, or extruded before tableting. The final table or capsule formulation may contain one or more layers and may be coated or uncoated; it may even be encapsulated in a particle, such as a polymeric particle or a liposomal particle.

Solid formulations containing the compound(s) for oral administration may be formulated to be immediate and/or modified release. Modified release formulations include delayed, sustained, pulsed, controlled, targeted and programmed release formulations.

b. Parenteral Formulations

In some forms, the pharmaceutical formulation containing one or more the disclosed compounds can be in a form suitable for administration directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intramuscular, and subcutaneous delivery. Suitable means for parenteral administration include needle (including microneedle) injectors, needle-free injectors, and infusion techniques.

For example, the pharmaceutical formulation containing one or more the compounds is in a form suitable for intramuscular administration, intravenous administration, intraperitoneal administration, or subcutaneous administration, or a combination thereof.

Parenteral formulations containing the compound(s) described herein are typically aqueous solutions which can contain excipients such as salts, carbohydrates and buffering agents (e.g., from about pH 6.5 to about pH 8.0, from about pH 6.5 to about pH 7.4, from about pH 6.5 to about pH 7.0, from about pH 7.0 to pH 8.0, or from about pH 7.0 to about pH 7.4), but, for some applications, they may be more suitably formulated as a sterile aqueous solution or as a dried form to be used in conjunction with a suitable vehicle such as sterile, pyrogen-free water.

The liquid formulations containing the compound(s) for parenteral administration may be a solution, a suspension, or an emulsion.

The liquid pharmaceutically acceptable carrier forming the parenteral formulation containing the compound(s) can include one or more physiologically compatible buffers, such as a phosphate buffers. One skilled in the art can readily determine a suitable saline content and pH for an aqueous carrier for administration (e.g., from about pH 6.5 to about pH 8.0, from about pH 6.5 to about pH 7.4, from about pH 6.5 to about pH 7.0, from about pH 7.0 to pH 8.0, or from about pH 7.0 to about pH 7.4).

Liquid formulations containing the compound(s) for parenteral administration may include one or more suspending agents, such as cellulose derivatives, sodium alginate, polyvinylpyrrolidone, gum tragacanth, or lecithin. The liquid formulations may also include one or more preservatives, such as ethyl or n-propyl p-hydroxybenzoate.

In some forms, the liquid formulation containing the compound(s) contains one or more solvents that are low toxicity organic (i.e., nonaqueous) class 3 residual solvents, such as ethanol, acetone, ethyl acetate, tetrahydofuran, ethyl ether, and propanol, and a combination thereof. Any such solvents included in the liquid formulation should not detrimentally react with the compound(s) and any additional active agents when present in the liquid formulation. Solvents such as freon, alcohol, glycol, polyglycol, or fatty acid, can also be included in the liquid formulation containing the compound(s) as desired to increase the volatility of the solution or suspension.

Liquid formulations containing the compound(s) for parenteral administration may also contain minor amounts of polymers, surfactants, or other pharmaceutically acceptable excipients known to those in the art. In this context, “minor amounts” means an amount that is sufficiently small to avoid adversely affecting uptake of the compounds by the targeted cells, such as pituitary gonadotrophs.

The preparation of parenteral formulations containing the compound(s) is typically under sterile conditions, for example, by lyophilisation, which can be accomplished using standard pharmaceutical techniques known to those skilled in the art.

Formulations for parenteral administration containing the compound(s) may be formulated to provide immediate and/or modified release of the active agent. Modified release formulations include delayed, sustained, pulsed, controlled, targeted and programmed release formulations.

c. Pulmonary and Mucosal Formulations

In some forms, the pharmaceutical formulation containing one or more the disclosed compounds can be in a form suitable for pulmonary or mucosal administration. The administration can include delivery of the composition to the lungs, nasal, oral (sublingual, buccal), vaginal, or rectal mucosa.

For example, the compounds can be administered intranasally or by oral inhalation, such as in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (such as an atomiser using electrohydrodynamics to produce a fine mist), or nebuliser, with or without the use of a suitable propellant, such as water, ethanol-water mixture, 1,1,1,2-tetrafluoroethane or 1,1,1,2,3,3,3-heptafluoropropane. For intranasal or oral inhalation use, the powder may comprise a bioadhesive agent, for example, chitosan or cyclodextrin. The term aerosol as used herein refers to any preparation of a fine mist of particles, which can be in solution or a suspension, whether or not it is produced using a propellant. Aerosols can be produced using standard techniques, such as ultrasonication or high-pressure treatment.

The pressurized container, pump, spray, atomizer, or nebuliser contains a solution or suspension of one or more of the compounds including, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, a drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compounds described herein, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose, and trehalose.

A suitable solution formulation containing the compound(s) for use in an atomizer using electrohydrodynamics to produce a fine mist may contain from 1 μg to 20 mg of one or more of the compounds per actuation and the actuation volume may vary from 1 μl to 100 μl. A typical formulation may contain one or more of the compounds described herein, propylene glycol, sterile water, ethanol and sodium chloride. Alternative solvents that may be used instead of propylene glycol include glycerol and polyethylene glycol.

Suitable flavors, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using, for example, PGLA. Modified release formulations include delayed, sustained, pulsed, controlled, targeted, and programmed release formulations.

In the case of dry powder inhalers and aerosols, the dosage unit is determined by means of a valve which delivers a metered amount. Units in accordance with the compounds are typically arranged to administer a metered dose or “puff.” The overall daily dose will be administered in a single dose or, more usually, as divided doses throughout the day.

In some forms, the compounds can be formulated for pulmonary delivery, such as intranasal administration or oral inhalation. Carriers for pulmonary formulations can be divided into those for dry powder formulations and for administration as solutions. For administration via the upper respiratory tract, the formulation can be formulated into an aqueous solution, e.g., water or isotonic saline, buffered or un-buffered, or as an aqueous suspension, for intranasal administration as drops or as a spray. Such aqueous solutions or suspensions may be isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

In some forms, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In some forms, solvents that are low toxicity organic (i.e. nonaqueous) class 3 residual solvents, such as ethanol, acetone, ethyl acetate, tetrahydrofuran, ethyl ether, and propanol may be used for the formulations containing the compound(s). The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In some forms, the pharmaceutical formulations containing the compound(s) may contain minor amounts of polymers, surfactants, or other excipients well known to those of the art. In this context, “minor amounts” means no excipients are present that might affect or mediate uptake of the compounds by cells and that the excipients that are present in amount that do not adversely affect uptake of compounds by cells.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Non-aqueous suspensions of lipids can also be prepared in absolute ethanol using a reusable PARI LC Jet+ nebulizer (PARI Respiratory Equipment, Monterey, CA).

d. Topical Formulations

The compounds can be administered directly to the external surface of the skin or the mucous membranes (including the surface membranes of the nose, lungs and mouth), such that the compounds can cross the external surface of the skin or mucous membrane and enters the underlying tissues.

Formulations for topical administration generally contain a dermatologically acceptable carrier that is suitable for application to the skin, has good aesthetic properties, is compatible with the active agents and any other components, and will not cause any untoward safety or toxicity concerns.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse or a transdermal patch. Other suitable topical carriers include anhydrous liquid solvents such as oils, alcohols, and silicones (e.g., mineral oil, ethanol isopropanol, dimethicone, cyclomethicone, and the like); aqueous-based single phase liquid solvents (e.g., hydro-alcoholic solvent systems, such as a mixture of ethanol and/or isopropanol and water); and thickened versions of these anhydrous and aqueous-based single phase solvents (e.g. where the viscosity of the solvent has been increased to form a solid or semi-solid by the addition of appropriate gums, resins, waxes, polymers, salts, and the like). Examples of topical carrier systems useful in the present formulations are described in the following four references all of which are incorporated herein by reference in their entirety: “Sun Products Formulary” Cosmetics & Toiletries, vol. 105, pp. 122-139 (December 1990); “Sun Products Formulary,” Cosmetics & Toiletries, vol. 102, pp. 117-136 (March 1987); U.S. Pat. No. 5,605,894 to Blank et al., and U.S. Pat. No. 5,681,852 to Bissett.

Formulations containing the compound(s) for topical administration may be formulated to be immediate and/or modified release. Modified release formulations include delayed, sustained, pulsed, controlled, targeted and programmed release formulations. Thus, the compounds may be formulated as a solid, semi-solid, or thixotropic liquid for administration as an implanted depot providing modified release of the compounds. Examples of such formulations include drug-coated stents and poly(dl-lactic-coglycolic)acid (PGLA) microspheres.

### 3. Additional Active Agent(s)

In some forms, the pharmaceutical formulation can include one or more additional active agents that are different from the DHA derivatives, such as one or more additional anticancer agents. Anticancer agents that can be included in the pharmaceutical compositions or formulations are known, for example, see the National Cancer Institute database, “A to Z List of Cancer Drugs,” website cancer.gov/about-cancer/treatment/drugs.

Exemplary anticancer drugs that can be included in the pharmaceutical formulation containing the compound(s) include, but are not limited to, olaparib, abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); rituximab, cyclophosphamide, doxorubicin, vincristine, prednisolone (RCHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

### 4. Dosages/wt % Concentration

In some forms, the pharmaceutical formulation contains an effective amount of the compound(s) for ameliorating one or more symptoms associated with a cancer in a subject.

In some forms, the compound(s) is present in the pharmaceutical formulation in an effective amount to induce apoptosis of cancer cells in the subject. The cytotoxicity of the compounds against cancer cells can be evaluated using the IC₅₀ values against the cancer cells, measured using known methods, such as MTT assay. Whether apoptosis of cancer cells is induced in the subject may be identified by a change of at least 5% in relevant biomarker or gene expression profile in a biological sample of the subject and/or by the number of cancer cells and/or dead cells in a tumor sample of the subject, compared to the biomarker or gene expression profile in the biological sample of the subject and/or by the number of cancer cells and/or dead cells in the tumor sample of the subject, before treatment with the compound(s). Exemplary biomarkers for showing apoptosis of cancer cells includes, but are not limited to, HER2 for breast cancer; PSA for prostate cancer; carcinoembryonic antigen (CEA), squamous cell carcinoma antigen (SCC), neuron-specific enolase (NSE), cytokeratin 19 fragment (CYFRA), and pro-gastrin-releasing peptide (proGRP) for lung cancer, or others. Alternatively or additionally, whether apoptosis of cancer cells is induced may be identified by chromatin condensation and fragmentation; and/or cleavalges of PARP, caspase-9, and/or caspase-3. In some forms, the pharmaceutical formulation contains the compound(s) in an amount that is effective to induce apoptosis of cancer cells as shown by chromatin condensation and fragmentation; cleavalges of PARP, caspase-9, and/or caspase-3; and/or changes in the levels of proteins involved in apoptotic signaling using Western blotting.

In some forms, the compound(s) is present in the pharmaceutical formulation in an effective amount to inhibit the growth of a tumor by at least 40%, at least 50%, or at least 55% in the subject compared to the same tumor in a control subject. Inhibition of tumor can be determined by known methods, such as by measuring the size of an isolated solid tumor or measuring the tumor size in vivo using imaging. In some forms, the pharmaceutical formulation contains the compound(s) in an amount that is effective to inhibit the growth of a tumor by at least 40%, at least 50%, or at least 55% in the subject, compared to the same tumor in a control subject by the end of a monitoring time period, such as by the end of 21 days, 28 days, 31 days, 2 months, 3 months, 6 months, or a year, optionally without any toxicity as shown by a stable body weight of the subject having the tumor. A stable body weight refers to a body weight change of less than about 10% during and by the end of the monitoring time period.

The total concentration of the compound(s) in the pharmaceutical formulation can be at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %. The term “total concentration of the compound(s) in the pharmaceutical formulation” refers to the sum of the weight of all compounds(s) relative to the weight of the formulation.

In some forms, the total concentration of the compound(s) in the pharmaceutical formulation that is effective to ameliorate one or more symptoms associated with a cancer in a subject, can be at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

In some forms, the total concentration of the compound(s) in the pharmaceutical formulation that is effective to induce apoptosis of cancer cells in the subject can be at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

In some forms, the total concentration of the compound(s) in the pharmaceutical formulation that is effective to inhibit the growth of a tumor by at least 40%, at least 50%, or at least 55% in the subject compared to the same tumor in a control subject, by the end of a monitoring period, can be at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

In some forms, the pharmaceutical formulation containing the compound(s) can be provided in a unit dosage form. The dosage of the compounds in the pharmaceutical formulation in the unit dosage form can be in a range from about 0.002 mg to about 1 mg, in a range from about 0.006 mg to about 0.6 mg, in a range from about 0.01 mg to about 0.4 mg, in a range from about 0.02 mg to about 0.3 mg, or in a range from about 0.01 mg to about 0.2 mg.

### III. Methods for Synthesizing the DHA Derivatives

Methods for synthesizing DHA derivatives containing a stereospecific C-10 ether linkage and an aryl pharmacophore moiety. The disclosed methods are cost effective and allow for stereospecific (e.g., at C-10 position) synthesis of DHA derivatives. For example, the method can produce enantiopure C-10 DHA derivatives or a high percentage (e.g., more than 50%) of a C-10 enantiomer (e.g., alpha C-10 DHA derivatives).

For example, in some forms, when the synthesis method involves acetalization reaction using an acidic catalyst that transforms DHA to an oxonium ion, the size and acidity of the catalyst may affect the conformation of transition state and the diastereoselectivity of the product, and thus affect the formation of alpha/beta products. Temperature under which the acetalization reaction is performed may also affect the conformation of transition state and thereby affect the formation of alpha/beta products.

In some forms, the methods can produce the DHA derivatives of Formulae IVa, IVb, or IVc:

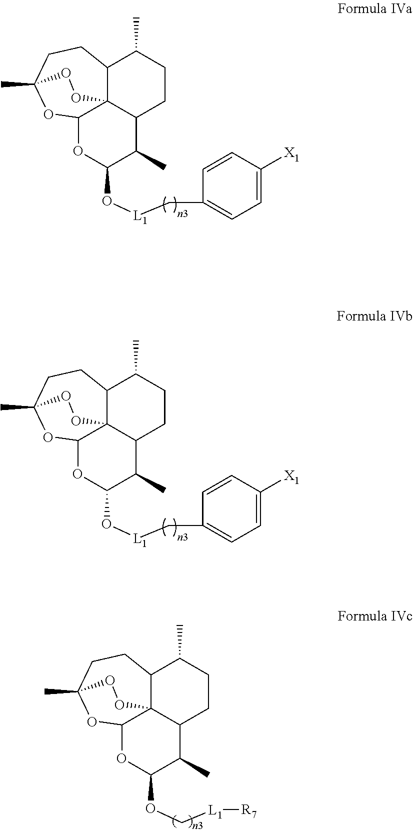

wherein: (i) X₁ can be a halide (e.g., bromide), hydroxyl, or thiol; (ii) n3 can be an integer from 0 to 5, from 1 to 5, from 1 to 4, from 1 to 3, or 1 or 2, such as 1; (iii) L₁ can be a bond,

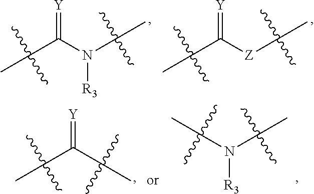

each occurrence of Y and Z are independently O or S (such as

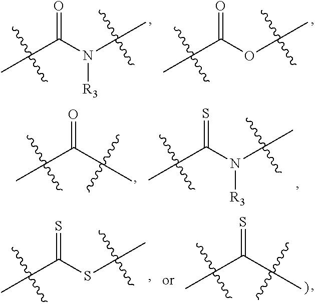

R₃ is hydrogen or an alkyl (such as an unsubstituted C1-C6 alkyl, an unsubstituted C1-C5 alkyl, an unsubstituted C1-C4 alkyl, an unsubstituted C1-C3 alkyl, a methyl, or an ethyl); and (iv) R₇ is an alkyl (e.g., methyl or ethyl) or

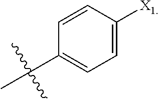

In some forms, L₁ can be a bond,

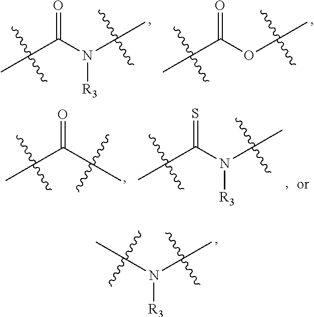

R₃ is hydrogen or an alkyl (such as an unsubstituted C1-C6 alkyl, an unsubstituted C1-C5 alkyl, an unsubstituted C1-C4 alkyl, an unsubstituted C1-C3 alkyl, a methyl, or an ethyl).

Generally, the method of synthesizing the DHA derivatives of Formula IVa and IVb includes: (a) maintaining a first reaction mixture at a temperature of 0° C. to room temperature for a time period of 2 hours to 15 hours, from 5 hours to 15 hours, or from 5 hours to 10 hours to form a first product containing the DHA derivatives of Formulae IVa and IVb. The first reaction mixture typically contains DHA, a first reactant having the structure of Formula V or V′, a catalyst, and an organic solvent,

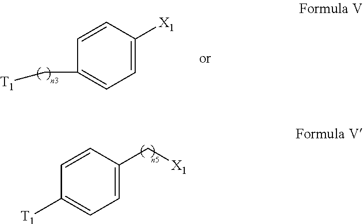

wherein: X₁ and n3 can be as defined for Formulae IVa and IVb; and T₁ can be hydroxyl, thiol (e.g., —SH), —SCF₃,

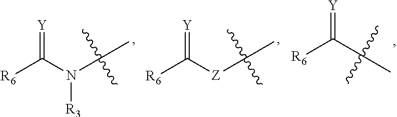

R₆ can be hydrogel or thiol (such as

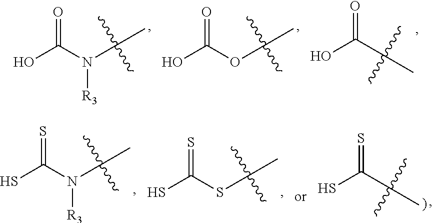

R₃ can be as defined above for Formulae IVa, IVb, and IVc.

In some forms, L₁ can be a bond, and T₁ can be hydroxyl.

In some forms, the first reaction mixture further includes a fourth reactant, such as CS₂ or CO₂.

Examples of catalyst that can be used in the first reaction mixture include, but are not limited to, BF₃-Et₂O, sulfuric acid, methanesulfonic acid, toluene-4-sulfonic acid, perchloric acid, amberlyst-15, dodecatungstophosphoric acid hydrate, and chloro-trimethyl-silane. For example, the catalyst used in the first reaction mixture is BF₃-Et₂O.

The organic solvent forming the first reaction mixture can be any suitable solvent that can dissolve DHA and the first reactant of Formula V. Examples of organic solvent in the first reaction mixture include, but are not limited to, alcohols (e.g., methanol, ethanol, n-propanol, isopropanol, etc.), dichloromethane (“DCM”), and benzene, and a combination thereof. For example, the organic solvent forming the first reaction mixture with DHA and the first reactant of Formula V is DCM.

Optionally, the method of synthesizing the DHA derivatives of Formulae IVa and IVb further includes: stirring the reaction mixture during step (a); quenching the reaction after step (a); drying the first product to remove the solvent; and/or purifying the first product. Each of these optional steps can be performed using methods known in the art.

For example, after the reaction in step (a) reaches completion, a quenching agent is added to the first reaction mixture to quench the reaction. The specific quenching agent can be selected based on the specific reactants. For example, to quench the reaction between DHA and the first reactant of Formula V in the present of the catalyst, a saturated aqueous sodium bicarbonate solution is added to the first reaction mixture.

For example, purification is performed to remove impurities (e.g., unreacted reactants) in the first product, and thereby obtain isolated DHA derivatives after the reaction in step (a). The purification can be performed using known methods, such as using extraction, washing, drying, filtering, or flash chromatography, or a combination thereof. For example, the first product formed in step (a) is quenched with saturated aqueous sodium bicarbonate. The layers are separated and the aqueous phase is extracted with a suitable organic solvent, such as DCM, and then washed with a suitable solvent, such as brine. The combined organic layer is dried with a suitable drying agent, such as Na₂SO₄, filtered, and evaporated in vacuo, followed by silica gel flash chromatography to obtain isolated DHA derivatives of Formula IVa, Formula IVb, and Formula IVc.

More specific conditions and reagents for the reaction to form exemplary DHA derivatives of Formulae IVa, IVb, and Formula IVc in step (a) are described in the Examples below.

In some forms, the methods can produce the DHA derivatives of an enantiopure C-10, such as a DHA derivative of Formula IIa,

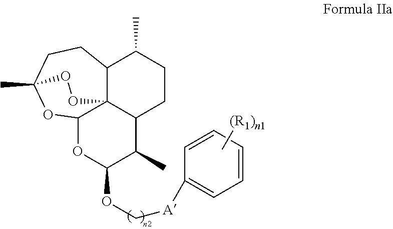

wherein: (i) R₁ can be an electron withdrawing group; (ii) n1 can be an integer from 0 to 5, from 1 to 5, from 1 to 4, from 1 to 3, or 1 or 2, such as 1; (iii) n2 can be an integer from 1 to 6, from 1 to 5, from 1 to 4, from 1 to 3, or 1 or 2; and (iv) A′ can be a bond,

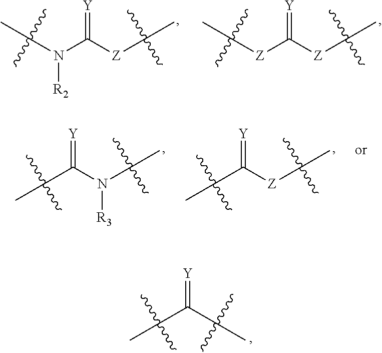

each occurrence of Y and Z are independently O or S (such as

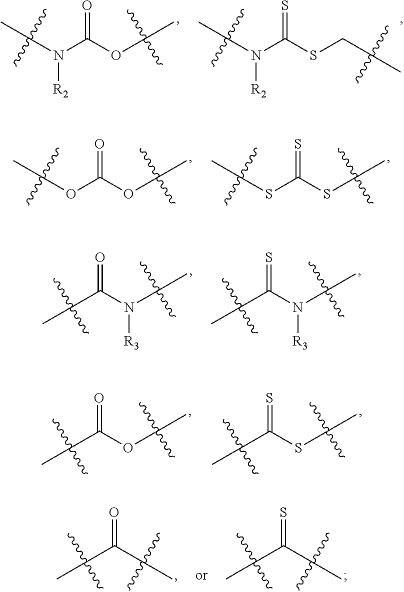

R₂ and R₃ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

Generally, the method of synthesizing the DHA derivatives of Formula IIa includes: (b) maintaining a second reaction mixture at a temperature of −10° C. to room temperature for a time period of 2 hours to 15 hours, from 5 hours to 15 hours, or from 5 hours to 10 hours to form a first intermediate product containing a first intermediate; and (c) converting the first intermediate to a second product containing the DHA derivative of Formula IIa.

The first intermediate can have the structure of Formula VI:

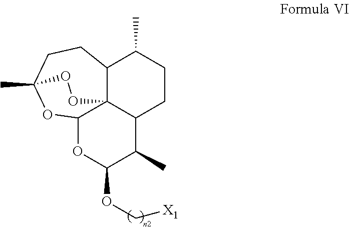

wherein n2 can be an integer from 1 to 6, from 1 to 5, from 1 to 4, from 1 to 3, or 1 or 2; and X₁ can be a halide (e.g., bromide, chloride, etc.). In some forms, X₁ can be bromide.

In some forms, the second reaction mixture typically contains DHA, a second reactant having the structure of Formula VII, a catalyst, and an organic solvent,

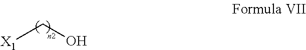

wherein n2 can be an integer from 1 to 6, from 1 to 5, from 1 to 4, from 1 to 3, or 1 or 2; and X₁ can be a halide (e.g., bromide, chloride, etc.). In some forms, X₁ can be bromide.

Examples of catalyst that can be used in the second reaction mixture include, but are not limited to, BF₃-Et₂O, sulfuric acid, methanesulfonic acid, toluene-4-sulfonic acid, perchloric acid, amberlyst-15, dodecatungstophosphoric acid hydrate, and chloro-trimethyl-silane. For example, the catalyst used in the second reaction mixture is BF₃-Et₂O.

The organic solvent forming the second reaction mixture can be any suitable solvent that can dissolve DHA and the second reactant of Formula VII. Examples of organic solvent in the second reaction mixture include, but are not limited to, alcohols (e.g., methanol, ethanol, n-propanol, isopropanol, etc.), dichloromethane (“DCM”), and benzene, and a combination thereof. For example, the organic solvent forming the second reaction mixture with DHA and the second reactant of Formula VII is an alcohol, such as ethanol.

Optionally, the method of synthesizing the DHA derivatives of Formula IIa can further include: stirring the reaction mixture during step (b); quenching the reaction after step (b) and before step (c); drying the first intermediate product to remove the solvent before step (c); and/or purifying the first intermediate product before step (c). Each of these optional steps can be performed using methods known in the art, such as those described above for synthesizing the DHA derivatives of Formulae IVa and IVb. For example, the first intermediate product formed in step (b) is quenched with saturated aqueous sodium bicarbonate. The layers are separated and the aqueous phase is extracted with a suitable organic solvent, such as ethanol, and then washed with a suitable solvent, such as brine. The combined organic layer is dried with a suitable drying agent, such as Na₂SO₄, filtered, and evaporated in vacuo, followed by silica gel flash chromatography to obtain isolated first intermediate of Formula VI.

More specific conditions and reagents for the reaction to form exemplary first intermediate of Formula VI in step (b) are described in the Examples below.

In step (c), the first intermediate of Formula VI can be converted to the second product containing the DHA derivative of Formula IIa using any suitable reactions. For example, as shown in Scheme 2, in step (c), the first intermediate of Formula VI reacts with an alkyl amine or ammonia to convert the halide of the first intermediate to an alkyl amino or aryl amino group, which then reacts with a reactant of Formula X, X′, or X″, optionally with carbon disulfide or carbon dioxide,

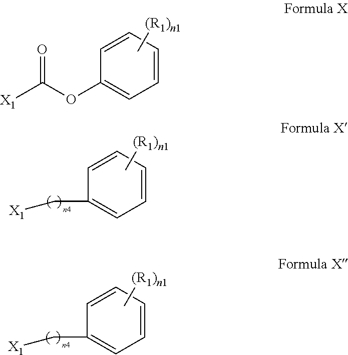

wherein n1 and R₁ can be as defined above for Formula IIa; n4 can be an integer from 1 to 5, from 1 to 4, from 1 to 3, or 1 or 2, such as 1; and X₁ can be a halide (e.g., bromide, chloride, etc.), to form the DHA derivative of Formula IIa, wherein A′ can be

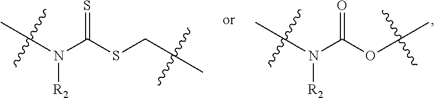

and R₂ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

It is understood that other suitable reactions can be performed in step (c) to convert the first intermediate of Formula VI to DHA derivative of Formula IIa with other A′ moieties, such as a bond,

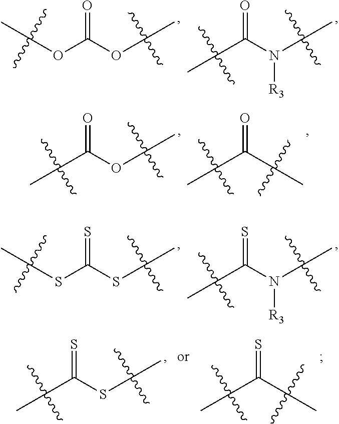

wherein R₂ and R₃ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

Optionally, the method of synthesizing the DHA derivatives of Formula IIa can further include: stirring the reaction mixture during step (c); quenching the reaction after step (c); drying the second product to remove the solvent; and/or purifying the second product. Each of these optional steps can be performed using methods known in the art, such as those described above for synthesizing the DHA derivatives of Formulae IVa, IVb, and IVc. For example, the second product formed in step (c) is diluted with a suitable solvent, such as DCM, and then washed with a suitable solvent, such as brine. The organic layer is dried with a suitable drying agent, such as Na₂SO₄, filtered, and evaporated in vacuo, followed by silica gel flash chromatography to obtain isolated DHA derivative of Formula IIa.

More specific conditions and reagents for the reaction to form exemplary DHA derivatives of Formula IIa in step (c) are described in the Examples below.

In some forms, the methods can produce a high percentage (e.g., a yield of at least 30%, at least 35%, at least 40%, at least 45%, or at least 50%) of a C-10 enantiomer, such as a DHA derivative of Formula IIb. For example, using the disclosed method, DHA derivative of Formula IIb is produced with a yield of at least 30%, at least 35%, at least 40%, at least 45%, or at least 50%, such as about 52%. In some forms, the yield of the DHA derivative of Formula IIb using the disclosed method is higher than that using previously reported method (see, e.g., Pranjal P. Bora, et al., Synthetic Communications, 2012, 42(8), 1218-1225), such as at least 20% higher, at least 30% higher, at least 40% higher, at least 50% higher, at least 100% higher, or at least 200% higher. For example, the yield of the DHA derivative of Formula IIb using the disclosed method (e.g., about 50%) is about 2-time higher than the yield of the alpha-C-10 DHA compound (about 28%) produced using the method described in Pranjal P. Bora, et al., Synthetic Communications, 2012, 42(8), 1218-1225.

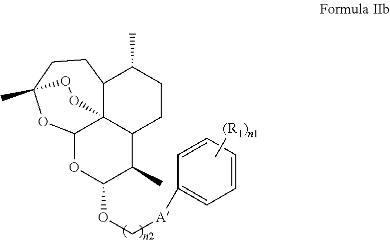

wherein: (i) R₁ can be an electron withdrawing group; (ii) n1 can be an integer from 0 to 5, from 1 to 5, from 1 to 4, from 1 to 3, or 1 or 2, such as 1; (iii) n2 can be an integer from 1 to 6, from 1 to 5, from 1 to 4, from 1 to 3, or 1 or 2; and (iv) A′ can be as defined above (such as a bond,

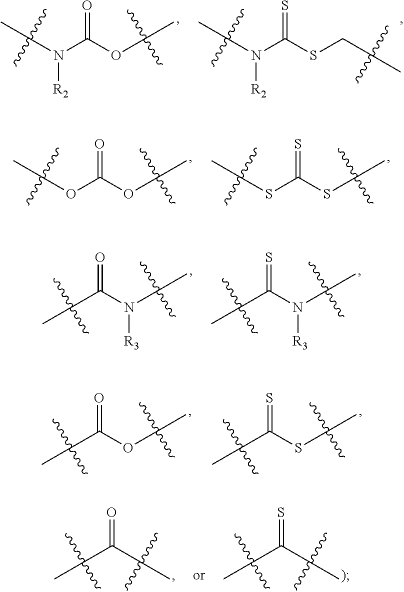

R₂ and R₃ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C₄ alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

Generally, the method of synthesizing a C-10 enantiomer, such as a DHA derivative of Formula IIb, with a high percentage includes: (d) maintaining a third reaction mixture at a temperature of −10° C. to room temperature for a time period of 2 hours to 15 hours, from 5 hours to 15 hours, or from 5 hours to 10 hours to form a second intermediate product containing a second intermediate; and (e) converting the second intermediate to a third product containing the DHA derivative of Formula IIb.

The second intermediate can have the structure of Formula VIII:

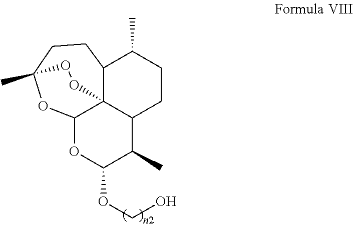

wherein n2 is as defined above for Formula IIb, e.g., an integer from 1 to 6, from 1 to 5, from 1 to 4, from 1 to 3, or 1 or 2, such as 2.

The third reaction mixture typically contains DHA, a third reactant having the structure of Formula IX, a catalyst, and an organic solvent,

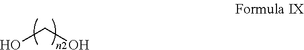

wherein n2 is as defined above for Formula IIb, e.g., an integer from 1 to 6, from 1 to 5, from 1 to 4, from 1 to 3, or 1 or 2, such as 2.

Examples of catalyst that can be used in the third reaction mixture include, but are not limited to, BF₃-Et₂O, sulfuric acid, methanesulfonic acid, toluene-4-sulfonic acid, perchloric acid, amberlyst-15, dodecatungstophosphoric acid hydrate, and chloro-trimethyl-silane. For example, the catalyst used in the third reaction mixture is BF₃-Et₂O.

The organic solvent forming the third reaction mixture can be any suitable solvent that can dissolve DHA and the third reactant of Formula IX. Examples of organic solvent in the second reaction mixture include, but are not limited to, alcohols (e.g., methanol, ethanol, n-propanol, isopropanol, etc.), dichloromethane (“DCM”), and benzene, and a combination thereof. For example, the organic solvent forming the third reaction mixture with DHA and the third reactant of Formula IX is an alcohol, such as ethanol. For example, the organic solvent forming the third reaction mixture with DHA and the third reactant of Formula IX is DCM.

Optionally, the method of synthesizing the DHA derivatives of Formula IIb with a high percentage can further include: stirring the reaction mixture during step (d); quenching the reaction after step (d) and before step (e); drying the second intermediate product to remove the solvent prior to step (e); and/or purifying the second intermediate product prior to step (e). Each of these optional steps can be performed using methods known in the art, such as those described above for synthesizing the DHA derivatives of Formulae IVa and IVb. For example, the second intermediate product formed in step (d) is quenched with saturated aqueous sodium bicarbonate. The layers are separated, and the aqueous phase is extracted with a suitable organic solvent, such as ethanol, and then washed with a suitable solvent, such as brine. The combined organic layer is dried with a suitable drying agent, such as Na₂SO₄, filtered, and evaporated in vacuo, followed by silica gel flash chromatography to obtain isolated second intermediate of Formula VIII.

More specific conditions and reagents for the reaction to form exemplary second intermediate of Formula VIII in step (d) are described in the Examples below.

In step (e), the second intermediate of Formula VIII can be converted to the third product containing the DHA derivative of Formula IIb using any suitable reactions. For example, as shown in Scheme 3, in step (e), the second intermediate of Formula VIII reacts with an alkyl amine and p-toluenesulfonyl chloride to convert the hydroxyl of the second intermediate to a tosylate. The tosylate can react with an alkyl amine to convert the tosylate to an alkyl amino group, which then reacts with a reactant of Formula X, X′, or X″, optionally with carbon disulfide or carbon dioxide,

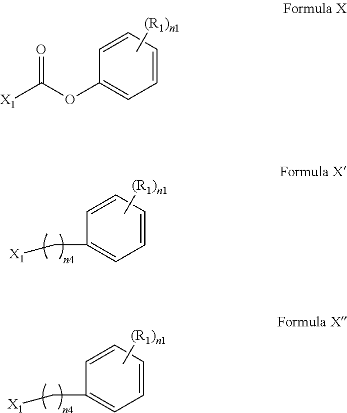

wherein n1 and R₁ can be as defined above for Formula IIb; and X₁ can be a halide (e.g., bromide, chloride, etc.), to form the DHA derivative of Formula IIb, wherein A′ can be

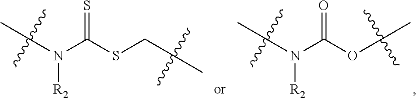

and R₂ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

It is understood that other suitable reactions can be performed in step (e) to convert the second intermediate of Formula VIII to DHA derivative of Formula IIb with other A′ moieties, such as a bond,

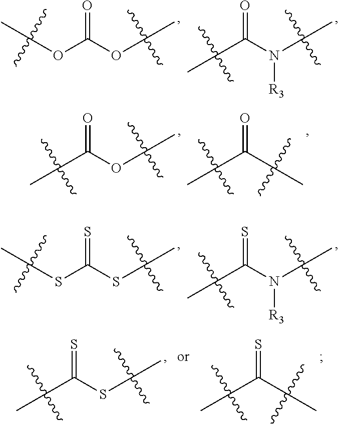

wherein R₂ and R₃ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

In some forms, the third product formed in step (e) further contains a DHA derivative of Formula IIa. In these forms, the percentage of the DHA derivative of Formula IIa is less than the percentage of the DHA derivative of Formula IIb. For example, in the third product formed in step (e), the DHA derivative of Formula IIb has a yield of at least 30%, at least 35%, at least 40%, at least 45%, or at least 50%, such as about 52%, and the yield of DHA derivative of Formula IIa is at least 10% less, at least 20% less, at least 40% less, at least 50%, or at least 80% less than that of the DHA derivative of Formula IIb.

Optionally, the method of synthesizing the DHA derivatives of Formula IIb can further include: stirring the reaction mixture during step (e); quenching the reaction after step (e); drying the third product to remove the solvent; and/or purifying the third product. Each of these optional steps can be performed using methods known in the art, such as those described above for synthesizing the DHA derivatives of Formulae IVa and IVb. For example, the third product formed in step (c) is diluted with a suitable solvent, such as DCM, and then washed with a suitable solvent, such as brine. The organic layer is dried with a suitable drying agent, such as Na₂SO₄, filtered, and evaporated in vacuo, followed by silica gel flash chromatography to obtain isolated DHA derivative of Formula IIb.

More specific conditions and reagents for the reaction to form exemplary DHA derivatives of Formula IIb in step (e) are described in the Examples below.

### IV. Methods for Using the DHA Derivatives

In some forms, the disclosed DHA derivatives have anticancer properties and thereby can be used in methods for treating a cancer in a subject in need thereof. In some forms, the DHA derivatives can be used in methods for treating cancer cells ex vivo or in a subject in need thereof.

It will be appreciated that the disclosed methods can be methods of treatment of the symptoms and conditions described herein. “Treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### A. Treating Cancer

Methods of using the DHA derivatives for treating a cancer in a subject in need thereof are disclosed. Generally, the method includes (i) administering to the subject a pharmaceutical formulation containing one or more of the DHA derivatives described above. The administration step can occur one or more times.

The subject can be a mammal, such as a human, a dog, a cat, a rat, a monkey, rabbits, guinea pigs, etc., that is in need of cancer treatment. In some forms, the subject can be exhibiting symptoms of or diagnosed with cancer.

The pharmaceutical formulation can be administered by oral administration, parenteral administration, mucosal administration, or topical administration, or a combination thereof. The compound(s) can be administered by a medical professional or the subject being treated (e.g. self-administration).

### 1. Cancers

The cancer being treated using the disclosed methods can be tumors, such as tumors of the reproductive system (e.g., ovarian cancer), tumors of the gastrointestinal tract (e.g., colon cancer), hematopoietic and lymphoid malignancies (tumors that affect the blood, bone marrow, lymph, and lymphatic system). Exemplary cancers that can be treated using the disclosed methods include, but are not limited to, tumors located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax, and genito-urinary apparatus.

In some forms, the cancer can be an ovarian cancer or a colon cancer. In some forms, the cancer can be a Cisplatin resistant cancer, such as Cisplatin resistant ovarian cancer.

In some forms, the cancer can be AIDS-related malignant tumors, anal cancer, astrocytoma, cancer of the biliary tract, cancer of the bladder, bone cancer, brain stem glioma, brain tumors, breast cancer, cancer of the renal pelvis and ureter, primary central nervous system lymphoma, central nervous system lymphoma, cerebellar astrocytoma, brain astrocytoma, cancer of the cervix, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood brain astrocytoma, childhood extracranial germ cell tumors, childhood Hodgkin's disease, childhood Hodgkin's lymphoma, childhood visual pathway and hypothalamic glioma, childhood lymphoblastic leukemia, childhood medulloblastoma, childhood non-Hodgkin's lymphoma, childhood supratentorial primitive neuroectodermal and pineal tumors, childhood primary liver cancer, childhood rhabdomyosarcoma, childhood soft tissue sarcoma, childhood visual pathway and hypothalamic glioma, chronic lymphocytic leukemia, chronic myeloid leukemia, cancer of the colon, cutaneous T-cell lymphoma, endocrine pancreatic islet cells carcinoma, endometrial cancer, ependymoma, epithelial cancer, cancer of the esophagus, Ewing's sarcoma and related tumors, cancer of the exocrine pancreas, extracranial germ cell tumor, extragonadal germ cell tumor, extrahepatic biliary tract cancer, cancer of the eye, breast cancer in women, Gaucher's disease, cancer of the gallbladder, gastric cancer, gastrointestinal carcinoid tumor, gastrointestinal tumors, germ cell tumors, gestational trophoblastic tumor, tricoleukemia, head and neck cancer, hepatocellular cancer, Hodgkin's disease, Hodgkin's lymphoma, hypergammaglobulinemia, hypopharyngeal cancer, intestinal cancers, intraocular melanoma, islet cell carcinoma, islet cell pancreatic cancer, Kaposi's sarcoma, cancer of kidney, cancer of the larynx, cancer of the lip and mouth, cancer of the liver, cancer of the lung, lymphoproliferative disorders, macroglobulinemia, breast cancer in men, malignant mesothelioma, malignant thymoma, medulloblastoma, melanoma, mesothelioma, occult primary metastatic squamous neck cancer, primary metastatic squamous neck cancer, metastatic squamous neck cancer, multiple myeloma, multiple myeloma/plasmatic cell neoplasia, myelodysplastic syndrome, myelogenous leukemia, myeloid leukemia, myeloproliferative disorders, paranasal sinus and nasal cavity cancer, nasopharyngeal cancer, neuroblastoma, non-Hodgkin's lymphoma during pregnancy, non-melanoma skin cancer, non-small cell lung cancer, metastatic squamous neck cancer with occult primary, buccopharyngeal cancer, malignant fibrous histiocytoma, malignant fibrous osteosarcoma/histiocytoma of the bone, epithelial ovarian cancer, ovarian germ cell tumor, ovarian low malignant potential tumor, pancreatic cancer, paraproteinemias, purpura, parathyroid cancer, cancer of the penis, phaeochromocytoma, hypophysis tumor, neoplasia of plasmatic cells/multiple myeloma, primary central nervous system lymphoma, primary liver cancer, prostate cancer, rectal cancer, renal cell cancer, cancer of the renal pelvis and ureter, retinoblastoma, rhabdomyosarcoma, cancer of the salivary glands, sarcoidosis, sarcomas, skin cancer, small cell lung cancer, small intestine cancer, soft tissue sarcoma, squamous neck cancer, stomach cancer, pineal and supratentorial primitive neuroectodermal tumors, T-cell lymphoma, testicular cancer, thymoma, thyroid cancer, transitional cell cancer of the renal pelvis and ureter, transitional renal pelvis and ureter cancer, trophoblastic tumors, cell cancer of the renal pelvis and ureter, cancer of the urethra, cancer of the uterus, uterine sarcoma, vaginal cancer, optic pathway and hypothalamic glioma, cancer of the vulva, Waldenstrom's macroglobulinemia, Wilms' tumor and any other hyperproliferative disease, as well as neoplasia, located in the system of a previously mentioned organ.

### 2. Effective Amount/Dosage

Generally, following the administration step of the disclosed method, the pharmaceutical formulation is administered in an effective amount to ameliorate one or more symptoms associated with a cancer in a subject.

For example, the method includes only a single administration of the pharmaceutical formulation, wherein following the administration step, an effective amount of the compound(s) to ameliorate one or more symptoms associated with the cancer in the subject is administered to the subject.

For example, the method includes more than one step of administering to the subject the pharmaceutical formulation, wherein following all of the administration steps, an effective amount of the compound(s) to ameliorate one or more symptoms associated with the cancer in the subject is administered to the subject.

In some forms, following the administration step or all of the administration steps (when more than one administration is performed), the pharmaceutical formulation is administered in an effective amount to inhibit the growth of a tumor by at least 40%, at least 50%, or at least 55% in the subject compared to the same tumor in a control subject or compared to the subject before administration of the pharmaceutical formulation. Whether tumor growth is inhibited may be identified by a variety of diagnostic manners known to one skill in the art including. For example, tumor growth inhibition can be identified by observation of the reduction in size or number of tumor masses, in comparison with the same type of tumor in a control subject, using imaging, by the end of a certain time period following administration of the effective amount of pharmaceutical formulation. Imaging suitable for measuring the size or number of tumor masses include, but are not limited to, transrectal ultrasound, MRI, computerized tomography (“CT”) scan, positron emission tomography (“PET”) imaging, multiparametric ultrasound (“US”), or a combination thereof, for example, PET-CT, PET-MRI, MRI-US, etc. For example, tumor growth inhibition can be identified by counting the number of dead cells in a tumor tissue of a subject by the end of a certain time period following administration of the effective amount of pharmaceutical formulation, in comparison with a tumor tissue of the same type and size in the subject prior to administration of the pharmaceutical formulation, using high-content analysis of cells.

In some forms, following the administration step or all of the administration steps (when more than one administration is performed), the pharmaceutical formulation is administered in an effective amount to reduce the level of a biomarker associated with a cancer in the blood of the subject by at least 5%, at least 10%, at least 15%, at least 20%, at least 25%, or at least 30% compared to the level of the biomarker in the blood of the subject before treatment. For example, the pharmaceutical formulation is administered in an effective amount to reduce the level of a biomarker associated with a lung cancer (e.g. carcinoembryonic antigen (CEA), squamous cell carcinoma antigen (SCC), neuron-specific enolase (NSE), cytokeratin 19 fragment (CYFRA), and pro-gastrin-releasing peptide (proGRP)) in the blood of the subject by at least 5%, at least 10%, at least 15%, at least 20%, at least 25%, or at least 30% compared to the level of the biomarker in the blood of the subject before treatment.

Administering an effective amount of the pharmaceutical formulation can be achieved in a single administration step or using multiple administration steps. For example, if the unit dosage form contains an effective amount of the compound(s) to inhibit the growth of a tumor by at least 40%, at least 50%, or at least 55% in the subject and/or reduce the level of a biomarker associated with a cancer in the blood of the subject, then the method only requires a single administration step. Alternatively, if the unit dosage form contains less than the required effective amount of the compound(s) to inhibit the growth of a tumor by at least 40%, at least 50%, or at least 55% in the subject and/or induce apoptosis of cancer cells, then the method involves at least two steps of administering the pharmaceutical formulation, and optionally more than two steps of administering the pharmaceutical formulation to the subject until an effective amount of the pharmaceutical formulation is administered to the subject to inhibit the growth of a tumor by at least 40%, at least 50%, or at least 55% in the subject and/or to reduce the level of a biomarker associated with a cancer in the blood of the subject. When multiple administration steps are needed to administer an effective amount of the pharmaceutical formulation to the patient, each administration step may administer the same dosage or different dosages of the pharmaceutical formulation to the patient.

When multiple administration steps are needed to administer an effective amount of the pharmaceutical formulation to the patient, the administration steps may be performed regularly or irregularly. For example, the administration steps are performed at a suitable frequency, such as every hour, every 2 hours, every 5 hours, every 8 hours, every day, every 2 days, every 3 days, every 5 days, every 7 days, every 10 days, every two weeks, or every month. For example, the administration step is performed every hour, every 2 hours, every 5 hours, every 8 hours, every day, every 2 days, every 3 days, every 5 days, every 7 days, every 10 days, every two weeks, or every month for a period between one day and 6 months, between one day and 3 months, between one and thirty days, between one and ten days, between one and three days, between one and two days, or for one day. Alternatively, the administration may be performed irregularly, for example, the administration step is performed 1 day after the first administration, then 2 days after the second administration, then 5 days after the third administration, then 7 days after the fourth administration, and then 30 days after the fifth administration. The time interval between administrations is determined based on the patient's needs.

In some forms, following a single administration or multiple administrations, the effective amount of compound(s) that is administered to the subject to ameliorate one or more symptoms associated with a cancer in a subject, can be in a range from about 0.1 mg/kg to about 50 mg/kg, in a range from about 0.3 mg/kg to about 30 mg/kg, in a range from about 0.5 mg/kg to about 20 mg/kg, in a range from about 1 mg/kg to about 15 mg/kg, or in a range from about 0.5 mg/kg to about 10 mg/kg, such as about 3 mg/kg of the subject.

### 3. Optional Steps

a. Administering Additional Active Agent(s)

One or more active agents in addition to the compounds may be administered to the subject throughout the method or at different intervals during the method. For example, the one or more additional active agents is administered to the subject prior to, during, and/or subsequent to step (i). In some forms, the one or more additional active agents can be included in a pharmaceutical formulation containing the compound(s) and is administered to the subject simultaneously with the compound(s) in the pharmaceutical formulation in association with one or more pharmaceutically acceptable excipients. In some forms, the one or more additional active agents can be administered separately from the pharmaceutical formulation containing the compound(s).

In some forms, the one or more additional active agents are one or more anticancer agents described above, such as Cisplatin. The amount of the one or more additional anticancer agents required will vary from subject to subject according to their needs.

### B. Treating Cancer Cells

In some forms, the compounds can be used in a method for treating cancer cells in a subject in need thereof.

The method can follow the method step(s) described above, for example, administering to the subject the pharmaceutical formulation containing the compound(s), such as by oral administration, parenteral administration, mucosal administration, or topical administration, or a combination thereof. The administration step can occur one or more times to administer an effective amount of the compound(s) in the pharmaceutical formulation to kill cancer cells, depending on whether a unit dosage contains an effective amount of the compound(s) to kill the cancer cells. When multiple administrations are needed to achieve the required effective amount of the compound(s) in the subject, the dosage and frequency for each administration can follow the method described above.

In some forms, the method can include the additional step described above. For example, the user can administer one or more additional active agents to the subject prior to, during, and/or subsequent to administering the compound to the subject.

In some forms, the compound(s) can have an IC₅₀ value against the cancer cells comparable to (e.g., concentration in μM on the same order of magnitude) or lower than an IC₅₀ value of the parent DHA, tested under the same condition. In some forms, the compound(s) can have an IC₅₀ value against the cancer cells compared to (e.g., concentration in μM on the same order of magnitude) or lower than an IC₅₀ value of an existing anticancer drug (e.g., Cisplatin), tested under the same condition.

For example, the DHA derivatives can have an IC₅₀ value against the cancer cells, such as ovarian cancer cells (e.g., A2780 cells and/or A2780CisR cells) and/or colon cancer cells (e.g., HCT-116 cells), comparable to (e.g., concentration in μM on the same order of magnitude) or lower than (such as a concentration in μM that is at least 1 order of magnitude lower) that of parent DHA and/or Cisplatin. For example, the DHA derivatives can have an IC₅₀ value against A2780 cells that is comparable to an IC₅₀ value against A2780CisR cells, demonstrating their effectiveness in treating Cisplatin resistant cancer cells.

In some forms of the method, the compound(s) can have an IC₅₀ value against the cancer cells lower than an IC₅₀ value of the same compound against non-cancerous cells, tested under the same condition.

The cancer cells being treated in the subject can be the cancer cells of any one of the cancers described above. For example, the cancer cells can be ovarian cancer cells (e.g., A2780 cells, A2780CisR cells, etc.), or colon cancer cells (e.g., HCT-116 cells), or a combination thereof. In some forms, the cancer cells can be A2780 cells and/or A2780CisR cells. In some forms, the cancer cells can be HCT-116 cells.

When comparing the IC₅₀ values of the compound against cancer cells with the IC₅₀ values of the same compound against non-cancerous cells, the non-cancerous cells can be from any normal tissue of the subject, such as CCD-19Lu.

### 1. Effective Amount/Dosage

In some forms, following the administration step of the disclosed method, the pharmaceutical formulation is administered in an effective amount to induce apoptosis of cancer cells in a subject, compared to the subject before administered with the pharmaceutical formulation.

In some forms, following a single administration or multiple administrations, the effective amount of compound(s) that is administered to the subject to induce apoptosis of cancer cells in the subject, compared to the subject before administered with the pharmaceutical formulation, can be in a range from about 0.1 mg/kg to about 50 mg/kg, in a range from about 0.3 mg/kg to about 30 mg/kg, in a range from about 0.5 mg/kg to about 20 mg/kg, in a range from about 1 mg/kg to about 15 mg/kg, or in a range from about 0.5 mg/kg to about 10 mg/kg, such as about 3 mg/kg of the subject.

The disclosed compounds and methods can be further understood through the following enumerated paragraphs.
Paragraph 1. A compound having the structure of:

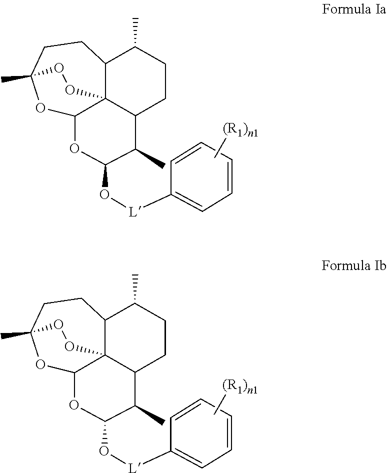

wherein:
(i) R₁ is an electron withdrawing group;
(ii) n1 is an integer from 0 to 5, from 1 to 5, from 1 to 4, from 1 to 3, or 1 or 2, such as 1; and
(iii) L′ is

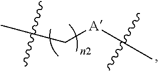

(a) n2 is an integer from 1 to 6, from 1 to 5, from 1 to 4, from 1 to 3, or 1 or 2; (b) A′ is a bond,

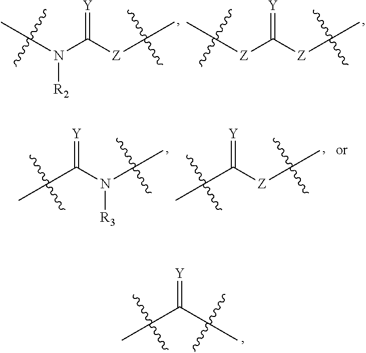

each occurrence of Y and Z are independently O or S (e.g. A′ is a bond,

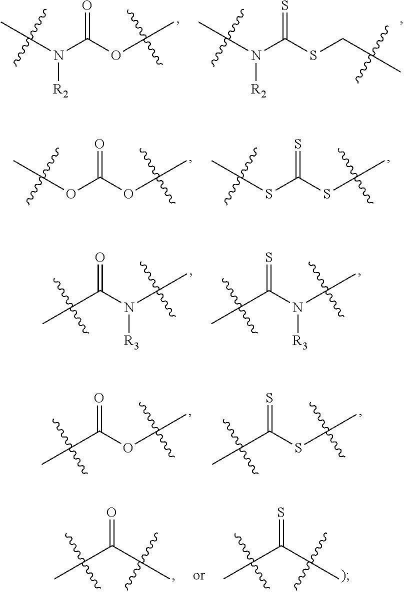

and (c) R₂ and R₃ can be independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).
Paragraph 2. The compound of paragraph 1, having the structure of:

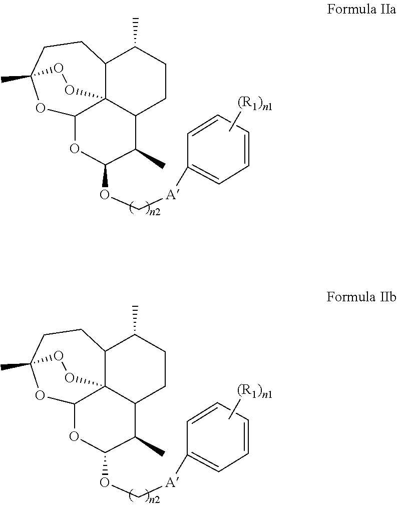

wherein:
(i) R₁ is an electron withdrawing group;
(ii) n1 is an integer from 0 to 5, from 1 to 5, from 1 to 4, from 1 to 3, or 1 or 2, such as 1;
(iii) n2 is an integer from 1 to 6, from 1 to 5, from 1 to 4, from 1 to 3, or 1 or 2; and
(iv) A′ is a bond,

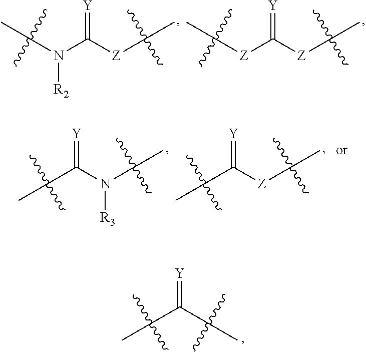

or each occurrence of Y and Z are independently O or S (e.g., A′ is a bond,

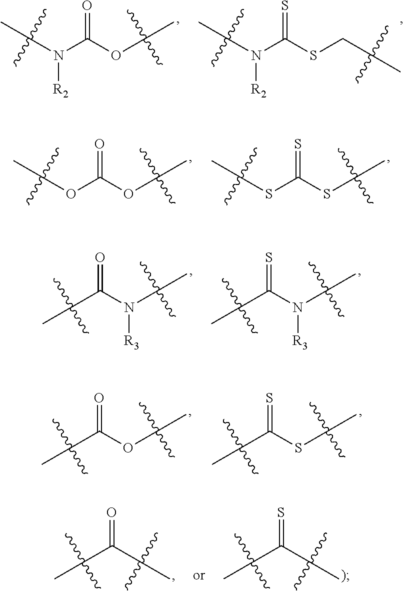

and (c) R₂ and R₃ are independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).
Paragraph 3. The compound of paragraph 1 or 2, A′ is a bond,

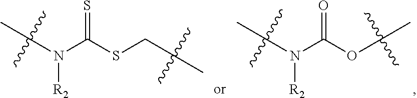

wherein R₂ is hydrogen or an alkyl, such as an unsubstituted C1-C6 alkyl, a substituted C1-C6 alkyl, an unsubstituted C1-C5 alkyl, an unsubstituted C1-C4 alkyl, an unsubstituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol (e.g., —SH), an alkoxy (e.g., —O-alkyl), a carboxylic acid (—COOH), a ketone (e.g., —C(═O)-alkyl), an aldehyde (e.g., —C(═O)H), or an ester (e.g., —C(═O)—O-alkyl).
Paragraph 4. The compound of any one of paragraphs 1-3, having the structure of:

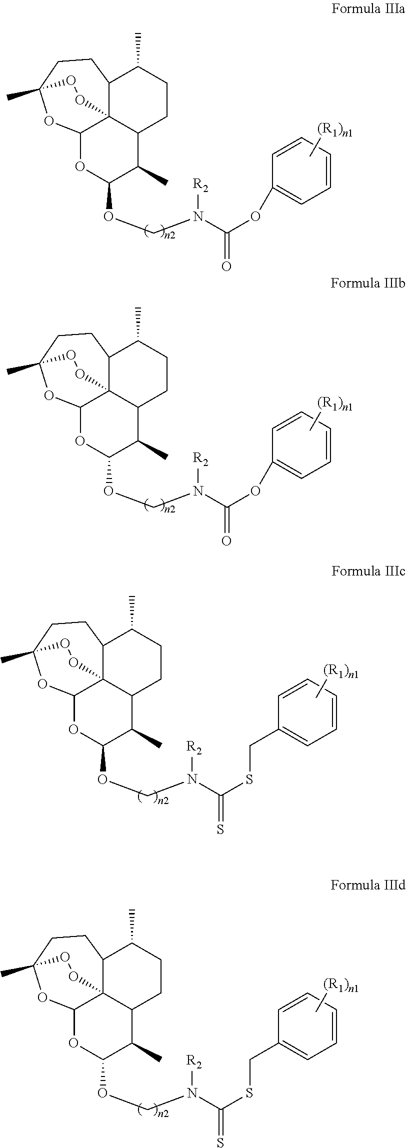

wherein:
(i) R₁ is an electron withdrawing group;
(ii) n1 is an integer from 0 to 5, from 1 to 5, from 1 to 4, from 1 to 3, or 1 or 2, such as 1;
(iii) n2 is an integer from 1 to 6, from 1 to 5, from 1 to 4, from 1 to 3, or 1 or 2; and
(iv) R₂ is independently hydrogen or an alkyl, such as an unsubstituted C1-C6 alkyl, a substituted C1-C6 alkyl, an unsubstituted C1-C5 alkyl, an unsubstituted C1-C4 alkyl, an unsubstituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol (e.g., —SH), an alkoxy (e.g., —O-alkyl), a carboxylic acid (—COOH), a ketone (e.g., —C(═O)-alkyl), an aldehyde (e.g., —C(═O)H), or an ester (e.g., —C(═O)—O-alkyl).
Paragraph 5. The compound of any one of paragraphs 1-4, wherein R₂ and R₃ are independently a methyl, an ethyl, a hexyl, or a hexyl substituted with a hydroxyl group.
Paragraph 6. The compound of any one of paragraphs 1-3, wherein A′ is a bond.
Paragraph 7. The compound of any one of paragraphs 1-6, wherein n1 and n2 are independently 1 or 2.
Paragraph 8. The compound of any one of paragraphs 1-7, wherein R₁ is a halide, an alkyl halide, a (trifluoromethyl)sulfanyl, a (trifluoromethyl)sulfinyl, a (trifluoromethyl)sulfonyl, a nitro, a nitrile, an aldehyde, a ketone, a carboxyl, a sulfonyl, a sulfinyl, or a sulfino.
Paragraph 9. The compound of any one of paragraphs 1-8, wherein R₁ is a halide or (trifluoromethyl)sulfanyl.
Paragraph 10. The compound of any one of paragraphs 1-9, having any one of the structures of:

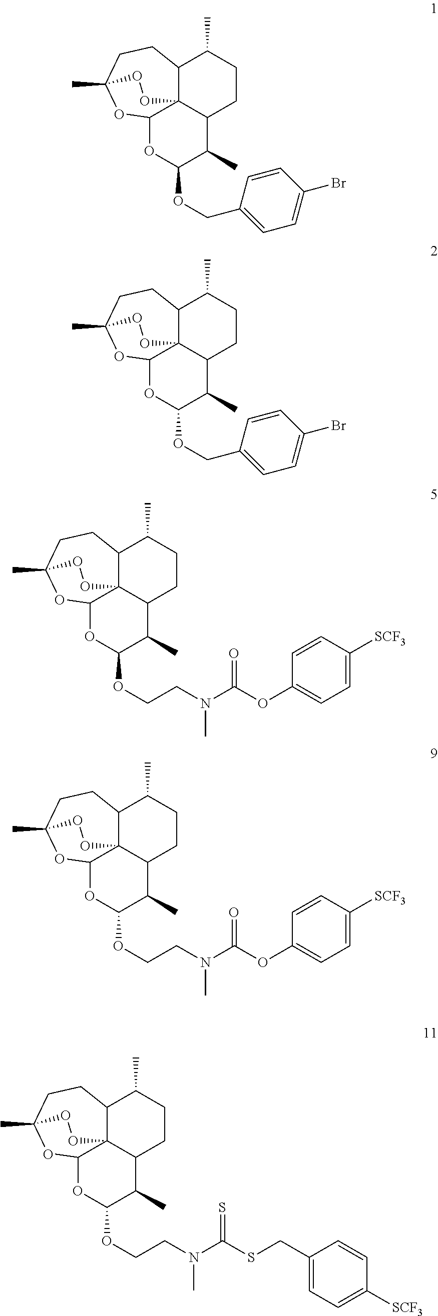

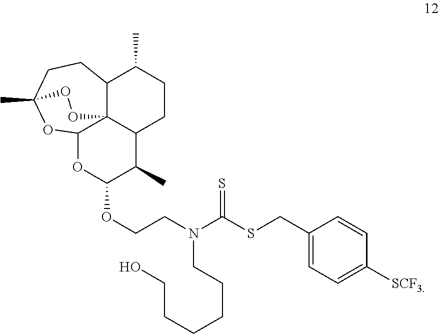

Paragraph 11. A pharmaceutical composition comprising one or more the compound(s) of any one of paragraphs 1-10, and a pharmaceutically acceptable carrier and/or excipient.
Paragraph 12. The pharmaceutical composition of paragraph 11, wherein the one or more compounds are in an effective amount to treat one or more symptoms associated with a cancer in a subject.
Paragraph 13. The pharmaceutical composition of paragraph 11 or 12, further comprising a second active agent that is different from the compound(s), optionally wherein the second active agent is an anticancer agent.
Paragraph 14. A method for treating a cancer in a subject comprising:
   (i) administering to the subject the pharmaceutical formulation of any one of paragraphs 11-13, wherein step (i) occurs one or more times.
Paragraph 15. The method of paragraph 14, wherein the method comprises only a single administration of the pharmaceutical formulation, wherein following the administration step, an effective amount of the compound(s) to treat one or more symptoms associated with the cancer in the subject is administered to the subject, or
   wherein the method comprises more than one step of administering to the subject the pharmaceutical formulation, wherein following all of the administration steps, an effective amount of the compound(s) to treat one or more symptoms associated with the cancer in the subject is administered to the subject.
Paragraph 16. The method of paragraph 14 or 15, wherein the subject is a mammal.
Paragraph 17. The method of any one of paragraphs 14-16, wherein the pharmaceutical formulation is administered by oral administration, parenteral administration, mucosal administration, or topical administration, or a combination thereof.
Paragraph 18. The method of any one of paragraphs 14-17, wherein the cancer is a Cisplatin resistant cancer, such as Cisplatin resistant ovarian cancer.
Paragraph 19. The method of any one of paragraphs 14-17, wherein the cancer is ovarian cancer or colon cancer.
Paragraph 20. The method of any one of paragraphs 14-19, further comprising administering to the subject a third active agent that is different from the compound(s), prior to, during, and/or subsequent to step (i), optionally wherein the third active agent is an anticancer agent.
Paragraph 21. A method for synthesizing DHA derivatives having the structures of Formula IVa and IVb,

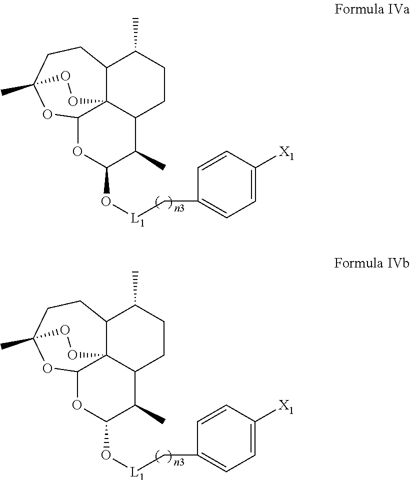

wherein: (i) X₁ is a halide (e.g., bromide); (ii) n3 is an integer from 0 to 5, from 1 to 5, from 1 to 4, from 1 to 3, or 1 or 2, such as 1; and (iii) L₁ is a bond,

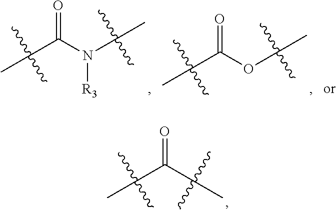

R₃ is hydrogen or an alkyl (such as an unsubstituted C1-C6 alkyl, an unsubstituted C1-C5 alkyl, an unsubstituted C1-C4 alkyl, an unsubstituted C1-C3 alkyl, a methyl, or an ethyl),
the method comprising: (a) maintaining a first reaction mixture at a temperature of 0° C. to room temperature for a time period of 2 hours to 15 hours, from 5 hours to 15 hours, or from 5 hours to 10 hours to form a first product comprising the DHA derivatives of Formula IVa and Formula IVb,
wherein the first reaction mixture comprises DHA, a first reactant having the structure of Formula V, a catalyst, and an organic solvent,

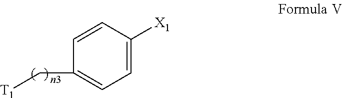

wherein: X₁ and n3 are as defined for Formula IVa and Formula IVb; and T₁ is hydroxyl,

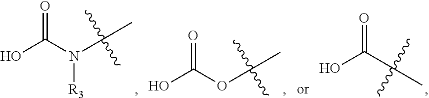

R₃ is as defined above for Formula IVa and Formula IVb.
Paragraph 22. The method of paragraph 21, wherein L₁ is a bond, and T₁ is hydroxyl.
Paragraph 23. The method of paragraph 21 or 22, further comprising: stirring the reaction mixture during step (a); quenching the reaction after step (a); drying the first product to remove the solvent; and/or purifying the first product.
Paragraph 24. A method for synthesizing a DHA derivative having the structure of Formula IIa,

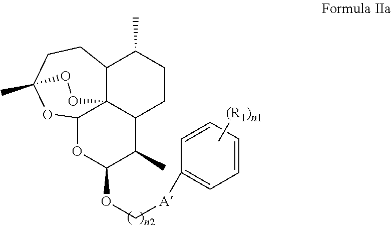

wherein: (i) R₁ is an electron withdrawing group; (ii) n1 is an integer from 0 to 5, from 1 to 5, from 1 to 4, from 1 to 3, or 1 or 2, such as 1; (iii) n2 is an integer from 1 to 6, from 1 to 5, from 1 to 4, from 1 to 3, or 1 or 2; and (iv) A′ is a bond,

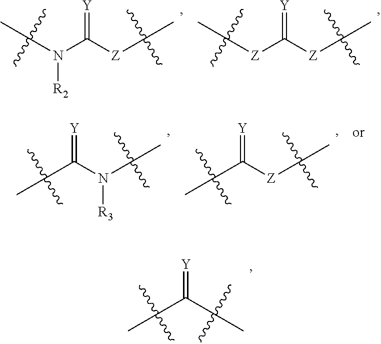

each occurrence of Y and Z are independently O or S (e.g., A′ is a bond,

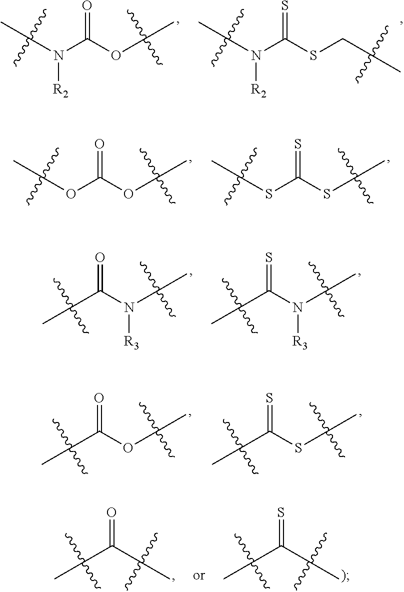

R₂ and R₃ are independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester),
the method comprising: (b) maintaining a second reaction mixture at a temperature of −10° C. to room temperature for a time period of 2 hours to 15 hours, from 5 hours to 15 hours, or from 5 hours to 10 hours to form a first intermediate product comprising a first intermediate of Formula VI; and (c) converting the first intermediate of Formula VI to a second product comprising the DHA derivative of Formula IIa,

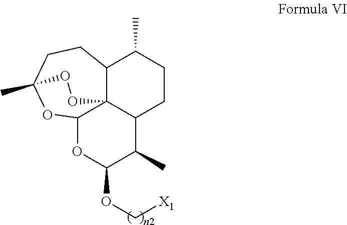

wherein the second reaction mixture comprises DHA, a second reactant having the structure of Formula VII, a catalyst, and an organic solvent,

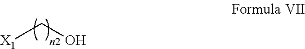

wherein n2 is an integer from 1 to 6, from 1 to 5, from 1 to 4, from 1 to 3, or 1 or 2; and X₁ is a halide.
Paragraph 25. A method for synthesizing a DHA derivative having the structure of Formula IIb,

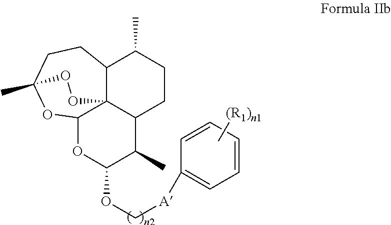

wherein: (i) R₁ is an electron withdrawing group; (ii) n1 is an integer from 0 to 5, from 1 to 5, from 1 to 4, from 1 to 3, or 1 or 2, such as 1; (iii) n2 is an integer from 1 to 6, from 1 to 5, from 1 to 4, from 1 to 3, or 1 or 2; and (iv) A′ is a bond,

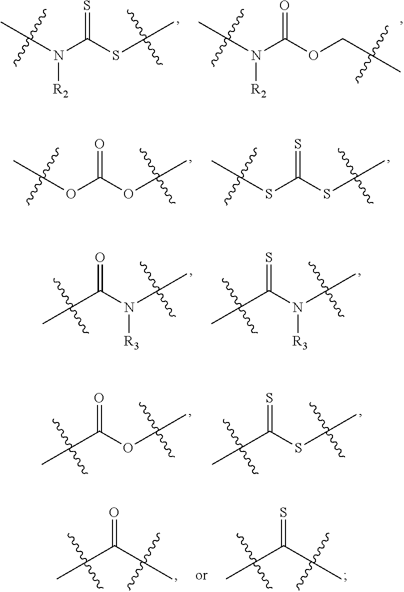

R₂ and R₃ are independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester),
the method comprising: (d) maintaining a third reaction mixture at a temperature of −10° C. to room temperature for a time period of 2 hours to 15 hours, from 5 hours to 15 hours, or from 5 hours to 10 hours to form a second intermediate product comprising a second intermediate of Formula VIII; and (e) converting the second intermediate of Formula VIII to a third product comprising the DHA derivative of Formula IIb,

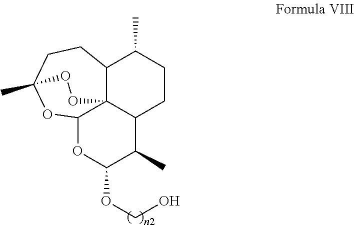

wherein the second reaction mixture comprises DHA, a third reactant having the structure of Formula IX, a catalyst, and an organic solvent,

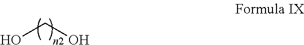

wherein n2 is an integer from 1 to 6, from 1 to 5, from 1 to 4, from 1 to 3, or 1 or 2.
Paragraph 26. The method of paragraph 25, wherein the third product further comprises a DHA derivative of Formula IIa.
Paragraph 27. The method of paragraph 25 or 26, wherein the DHA derivative of Formula IIb has a yield of at least 30%, at least 35%, at least 40%, at least 45%, or at least 50%.
Paragraph 28. The method of any one of paragraphs 24-27, wherein A′ is

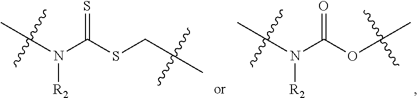

and R₂ is hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).
Paragraph 29. The method of any one of paragraphs 24-28, further comprising: stirring the second or third reaction mixture during step (b) or (d); quenching the reaction after step (b) or (d) and before step (c) or (e); drying the first or second intermediate product to remove the solvent after step (b) or (d) and before step (c) or (e); and/or purifying the first or second intermediate product after step (b) or (d) and before step (c) or (e).
Paragraph 30. The method of any one of paragraphs 24-29, further comprising: quenching the reaction after step (c) or (e); drying the second or third product to remove the solvent after step (c) or (e); and/or purifying the second or third product after step (c) or (e).
Paragraph 31. The method of any one of paragraphs 21-30, wherein the organic solvent in the first, second, and/or third reaction mixture is independently an alcohol (e.g., ethanol), benzene, or dichloromethane, or a combination thereof, such as ethanol or DCM.
Paragraph 32. The method of any one of paragraphs 21-30, wherein catalyst is BF₃-Et₂O, sulfuric acid, methanesulfonic acid, toluene-4-sulfonic acid, perchloric acid, amberlyst-15, dodecatungstophosphoric acid hydrate, or chloro-trimethyl-silane, such as BF₃-Et₂O.

The present invention will be further understood by reference to the following non-limiting examples.

### EXAMPLES

### Example 1. Dihydroartemisinin (“DHA”) Derivatives Show Anticancer Activity

### Materials and Methods

A series of DHA derivatives (e.g., compounds 1, 2, 5 and 9) were synthesized.

Compounds 1 and 2 with 4-bromobenzyl group were prepared using BF₃-Et₂O catalysed etherification, resulting in a mixture of 100-isomer 1 and 10α-isomer 2, which can be readily separated using flash column chromatography.

Treatment of DHA with bromoethanol in the presence of BF₃-Et₂O at −10° C. produced the stereospecific 100-isomer 3. Subsequence amination and carbamate formation produced compound 5. By replacing bromoethanol with ethylene glycol, 10α-isomer 9 was prepared following a similar synthetic route.

The detailed synthesis and structure characterization are described below.

### Synthesis of Compounds 1 and 2

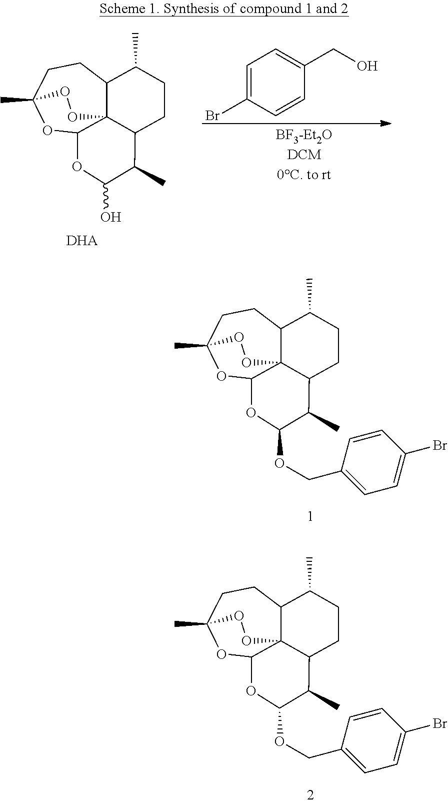

To a solution of dihydroartemisinin (0.68 g, 2.39 mmol) and 4-bromobenzyl alcohol (0.45 g, 2.39 mmol) in anhydrous DCM (20 mL) was added BF₃-Et₂O (0.45 mL, 3.58 mmol) at 0° C. The reaction mixture was stirred from 0° C. to room temperature for 8 h. The reaction was quenched with saturated aqueous sodium bicarbonate (100 mL), then the layers were separated, and the aqueous phase was extracted three times with DCM then washed with brine. The combined organic layer was dried with Na₂SO₄, filtered and evaporated in vacuo. Purification by silica gel flash chromatography afforded the desired benzyl compound 1 (0.37 g, 35%) and 2 (0.32 g, 29%).

Characterization of compound 1: ¹H NMR (600 MHz, Chloroform-d) δ 7.46 (d, J=8.2 Hz, 2H), 7.19 (d, J=8.1 Hz, 2H), 5.44 (s, 1H), 4.89 (d, J=3.5 Hz, 1H), 4.84 (d, J=12.5 Hz, 1H), 4.47 (d, J=12.5 Hz, 1H), 2.67 (tt, J=7.5, 3.4 Hz, 1H), 2.38 (td, J=14.0, 4.0 Hz, 1H), 2.05 (ddd, J=11.6, 5.0, 2.5 Hz, 1H), 1.91-1.86 (m, 1H), 1.82-1.75 (m, 2H), 1.62 (dt, J=9.8, 3.4 Hz, 2H), 1.53-1.48 (m, 1H), 1.45 (s, 3H), 1.31 (ddd, J=8.2, 6.3, 3.0 Hz, 1H), 1.26 (dd, J=11.4, 6.3 Hz, 2H), 0.94 (m, 7H). ¹³C NMR (151 MHz, CDCl₃) δ 137.36, 131.41, 128.92, 121.21, 104.17, 101.42, 88.02, 81.09, 69.02, 52.54, 44.34, 37.42, 36.41, 34.58, 30.87, 26.18, 24.67, 24.50, 20.32, 13.07. HR-MS (positive mode): calculated for [M+H]⁺ 453.1271; Found 453.1270.

Characterization of compound 2: ¹H NMR (600 MHz, Chloroform-d) δ 7.46 (d, J=8.3 Hz, 2H), 7.23 (d, J=8.2 Hz, 2H), 5.48 (s, 1H), 5.06 (d, J=5.0 Hz, 1H), 4.85 (d, J=12.2 Hz, 1H), 4.55 (d, J=12.2 Hz, 1H), 2.31 (ddd, J=14.7, 13.4, 3.9 Hz, 1H), 2.03 (ddd, J=14.6, 4.8, 3.2 Hz, 1H), 1.90 (ddt, J=13.6, 6.3, 3.6 Hz, 1H), 1.77-1.64 (m, 2H), 1.62-1.57 (m, 2H), 1.46 (m, 2H), 1.43 (s, 3H), 1.31-1.24 (m, 2H), 1.18 (d, J=7.2 Hz, 3H), 1.04-0.96 (m, 1H), 0.94 (d, J=5.7 Hz, 3H). ¹³C NMR (151 MHz, CDCl₃) δ 137.11, 131.43, 129.41, 121.41, 103.09, 102.15, 89.20, 81.64, 69.42, 51.85, 46.51, 39.77, 37.29, 36.50, 34.39, 31.63, 25.96, 24.68, 20.04, 19.49. HR-MS (positive mode): calculated for [M+H]⁺ 453.1271; Found 453.1268.

### Synthesis of Compound 5

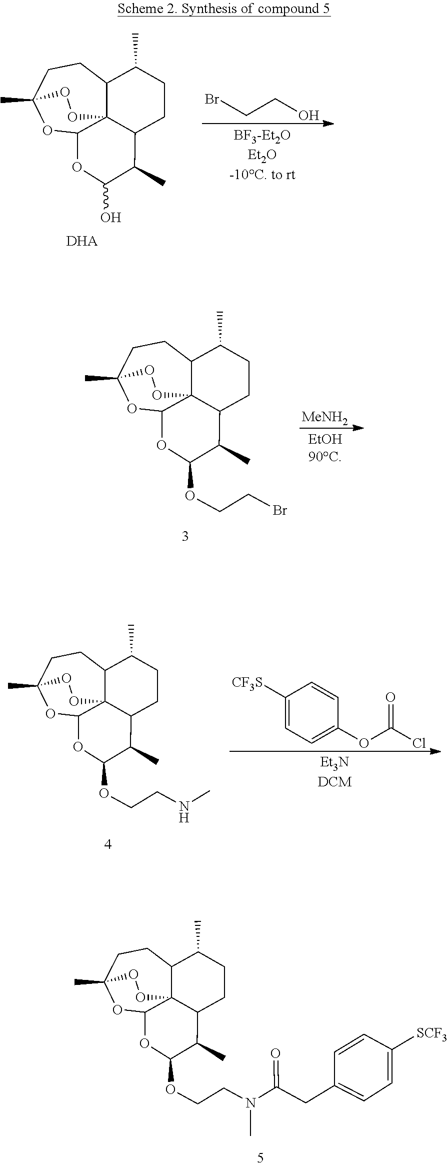

To a solution of dihydroartemisinin (3.0 g, 10.5 mmol) and bromoethanol (0.75 ml, 10.5 mmol) in anhydrous Et₂O (50 mL) was added BF₃-Et₂O (2 mL, 15.84 mmol) at −10° C. The reaction mixture was stirred from −10° C. to room temperature for 8 h. The reaction was quenched with saturated aqueous sodium bicarbonate (15 mL), then the layers were separated, and the aqueous phase was extracted three times with Et₂O then washed with brine. The combined organic layer was dried with Na₂SO₄, filtered and evaporated in vacuo. Purification by silica gel flash chromatography afforded the desired bromo compound 3 (2.53 g, 62%).

To a solution of bromo compound 3 (0.53 g, 1.35 mmol) in EtOH (10 mL) was added 33 wt % methylamine ethanol solution (5 mL) in seal-tube. The reaction was heated at 50° C. for 16 h. The reaction mixture was cooled to room temperature upon completion and concentrated in vacuo. Purification by silica gel flash chromatography afforded the desired methylamine compound 4 (345 mg, 75%).

To the solution of methylamine 4 (34 mg, 0.1 mmol) in DCM (5 mL) and triethylamine (21 μL, 148 mmol) was added 4-((trifluoromethyl)thio)phenyl carbonochloridate (30 mg, 0.12 mmol). The reaction mixture was stirred at room temperature for 4 h. The reaction mixture was diluted with DCM, then washed with brine, the organic layer was dried with Na₂SO₄, filtered and evaporated in vacuo to afford crude product, which was purified by silica gel flash chromatography to afford the desired carbamate compound 5 (53 mg, 94%).

Characterization of compound 3: ¹H NMR (600 MHz, Chloroform-d) δ 5.48 (s, 1H), 4.84 (dd, J=3.5, 0.9 Hz, 1H), 4.11 (ddd, J=11.7, 6.6, 5.4 Hz, 1H), 3.78 (dt, J=11.0, 5.3 Hz, 1H), 3.55-3.48 (m, 2H), 2.67-2.61 (m, 1H), 2.36 (dd, J=14.6, 13.5, 4.0 Hz, 1H), 2.06-2.01 (m, 1H), 1.93-1.85 (m, 2H), 1.77-173 (m, 1H), 1.67-1.63 (m, 1H), 1.48 (m, 2H), 1.43 (s, 3H), 135 (m, 1H), 1.23 (m, 1H), 0.96-0.88 (m, 7H). ¹³C NMR (151 MHz, CDCl₃) δ 104.07, 101.99, 88.09, 81.05, 68.12, 52.52, 44.30, 37.34, 36.35, 34.61, 31.39, 30.84, 26.10, 24.60, 24.32, 2033, 12.94. IR-MS (positive mode): calculated for [M+H]⁺ 391.1115; Found 391.1111.

Characterization of compound 4: ¹H NMR (600 MHz, Chloroform-d) δ 5.42 (s, 1H), 4.82 (dd, J=3.6, 1.0 Hz, 1H), 4.03 (dt, J=10.2, 4.9 Hz, 1H), 3.66-3.58 (m, 1H), 2.92-2.88 (m, 2H), 2.65 (m, 2H), 2.55 (s, 3H), 2.36 (ddd, J=14.6, 13.5, 4.0 Hz, 1H), 2.03 (ddd, J=14.6, 5.0, 3.0 Hz, 1H), 1.88 (m, 1H), 1.79-1.68 (m, 2H), 1.67-1.62 (m, 1H), 1.48 (m, 2H), 1.43 (s, 3H), 1.36 (m, 1H), 1.24 (m, 1H), 0.96-0.89 (m, 7H). ¹³C NMR (151 MHz, CDCl₃) δ 104.14, 102.34, 87.93, 81.00, 66.56, 52.51, 50.48, 44.30, 37.37, 36.38, 35.38, 34.57, 30.81, 26.13, 24.65, 24.54, 20.34, 13.03. HR-MIS (positive mode): calculated for [M+H]⁺ 342.2275; Found 342.2273.

Characterization of compound 5: ¹H NMR (600 MHz, Acetonitrile-d3) δ 7.76-7.71 (m, 2H), 7.29 (dd, J=15.6, 8.7 Hz, 2H), 5.42 (d, J=17.8 Hz, 1H), 4.76 (dd, J=6.5, 3.5 Hz, 1H), 4.04-3.92 (m, 1H), 3.68-3.49 (m, 2H), 3.12 (s, 1.5H, rotamer), 3.01 (s, 1.5H, rotamer), 2.56-2.46 (m, 1H), 2.32-2.24 (m, 1H), 2.09-2.02 (m, 1H), 1.88-1.66 (m, 3H), 1.55 (m, 1H), 1.44 (m, 2H), 1.35 (s, 3H), 1.28 (m, 1H), 1.20 (m, 1H), 0.95-0.88 (m, 7H). ¹³C NMR (151 MHz, CD₃CN) δ 154.88, 138.24, 123.75, 123.56, 104.26, 102.38, 88.23, 88.18, 81.37, 78.74, 78.52, 78.31, 66.05, 65.61, 53.08, 53.06, 49.45, 49.30, 44.82, 44.77, 37.63, 36.70, 35.32, 35.18, 34.92, 34.79, 31.41, 25.85, 25.09, 24.91, 24.75, 20.15, 12.90. 19F NMR (565 MHz, CDCl3) δ −43.07. HR-MS (positive mode): calculated for [M+H]⁺ 562.2081; Found 562.2084.

### Synthesis of Compound 9

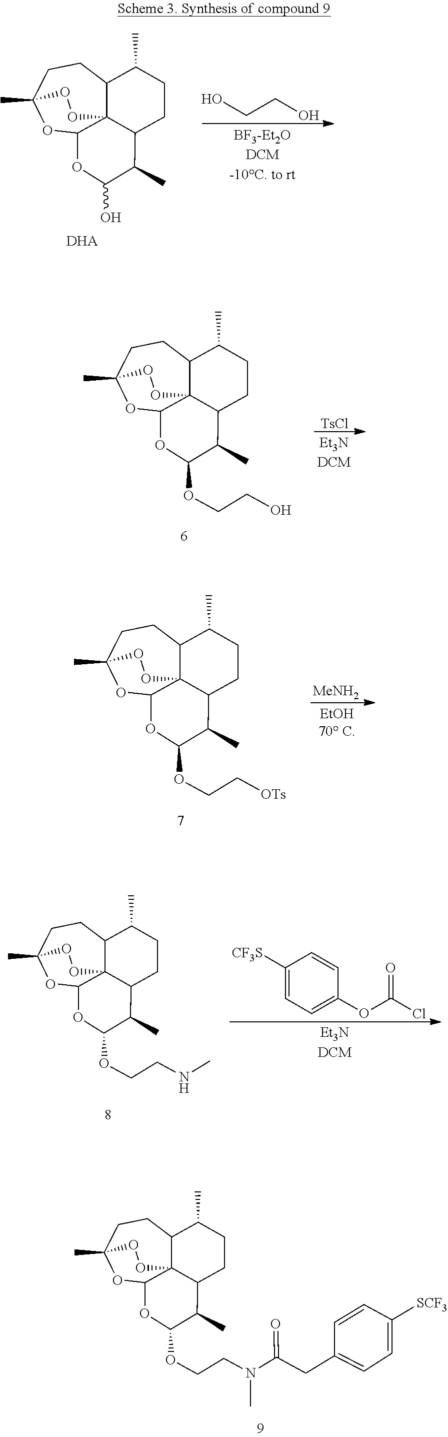

To a solution of dihydroartemisinin (1.17 g, 4.11 mmol) and ethylene glycol (0.22 mL, 4.11 mmol) in anhydrous DCM (30 mL) was added BF₃-Et₂O (0.8 mL, 6.18 mmol) at −10° C. The reaction mixture was stirred from −10° C. to room temperature for 8 h. The reaction was quenched with saturated aqueous sodium bicarbonate (30 mL), then the layers were separated, and the aqueous phase was extracted three times with DCM then washed with brine. The combined organic layer was dried with Na₂SO₄, filtered and evaporated in vacuo. Purification by silica gel flash chromatography afforded the desired hydroxyl compound 6 (0.43 g, 52%).

To the solution of hydroxyl compound 6 (370 mg, 1.13 mmol) in DCM (10 mL) with DMAP (cat.) and triethylamine (240 μL, 1.7 mmol) was added TsCl (255 mg, 1.34 mmol). The reaction mixture was stirred at room temperature for 8 h. The reaction mixture was diluted with DCM, then washed with brine, the organic layer was dried with Na₂SO₄, filtered and evaporated in vacuo to afford crude product, which was purified by silica gel flash chromatography to afford the desired tosylate compound 7 (528 mg, 97%).

To a solution of tosylate compound 7 (65 mg, 0.1 mmol) in EtOH (10 mL) was added 33 wt % methylamine ethanol solution (5 mL) in seal-tube. The reaction was heated at 80° C. for 16 h. The reaction mixture was cooled to room temperature upon completion and concentrated in vacuo. Purification by silica gel flash chromatography afforded the desired methylamine compound 8 (33 mg, 75%).

To the solution of methylamine 8 (62 mg, 0.18 mmol) in DCM (8 mL) and triethylamine (33 μL, 0.23 mmol) was added 4-((trifluoromethyl)thio) phenyl carbonochloridate (55 mg, 0.21 mmol). The reaction mixture was stirred at room temperature for 4 h. The reaction mixture was diluted with DCM, then washed with brine, the organic layer was dried with Na₂SO₄, filtered and evaporated in vacuo to afford crude product, which was purified by silica gel flash chromatography to afford the desired carbamate compound 9 (74 mg, 74%).

Characterization of compound 6: ¹H NMR (600 MHz, Chloroform-d) δ 5.36 (s, 1H), 4.46 (d, J=9.3 Hz, 1H), 3.88-3.81 (m, 2H), 3.78-3.68 (m, 2H), 3.10 (t, J=5.9 Hz, 1H), 2.48-2.42 (m, 1H), 2.37 (ddd, J=14.6, 13.4, 4.0 Hz, 1H), 2.02 (ddd, J=14.6, 5.0, 3.0 Hz, 1H), 1.88 (dddd, J=13.6, 6.7, 4.0, 3.0 Hz, 1H), 1.76 (dq, J=13.6, 3.7 Hz, 1H), 1.69 (dd, J=13.3, 3.3 Hz, 1H), 1.56 (dt, J=13.8, 4.6 Hz, 1H), 1.51-1.44 (m, 1H), 1.41 (s, 3H), 1.34-1.21 (m, 3H), 1.05-0.97 (m, 1H), 0.95 (d, J=6.1 Hz, 3H), 0.91 (d, J=7.1 Hz, 3H). ¹³C NMR (151 MHz, CDCl₃) δ 104.35, 100.58, 91.07, 80.21, 71.84, 62.37, 51.47, 45.29, 37.37, 36.16, 34.16, 32.44, 25.89, 24.68, 22.09, 20.21, 12.57 HR-MS (positive mode): calculated for [M−H₂O]⁺ 311.1853; Found 311.1855.

Characterization of compound 7: ¹H NMR (600 MHz, Chloroform-d) δ 7.76 (d, J=8.3 Hz, 2H), 7.34-7.29 (m, 2H), 5.26 (s, 1H), 4.39 (d, J=9.2 Hz, 1H), 4.15 (dd, J=5.7, 3.9 Hz, 2H), 4.03 (dt, J=11.9, 3.8 Hz, 1H), 3.69 (dt, J=11.6, 5.7 Hz, 1H), 2.41 (s, 3H), 2.36-2.25 (m, 2H), 2.00-197 (m, 1H), 1.88-1.82 (m, 1H), 171 (dq, J=13.5, 3.7 Hz, 1H), 1.65 (dq, J=13.4, 3.3 Hz, 1H), 1.49 (dt, J=14.0, 4.7 Hz, 1H), 1.45-1.39 (m, 1H), 1.37 (s, 3H), 1.32-1.16 (m, 3H), 1.00-0.93 (m, 1H), 0.92 (d, J=6.2 Hz, 3H), 0.79 (d, J=7.1 Hz, 3H). ¹³C NMR (151 MHz, CDCl₃) δ 144.68, 132.74, 129.71, 127.84, 116.35, 104.13, 100.11, 91.03, 80.17, 69.35, 65.98, 51.44, 45.07, 37.19, 36.12, 34.03, 32.39, 25.84, 24.52, 21.99, 21.50, 20.12, 12.22. HR-MS (positive mode): calculated for [M+H]⁺ 483.2047; Found 483.2048.

Characterization of compound 8: ¹H NMR (600 MHz, Chloroform-d) δ 5.37 (s, 1H), 4.47 (d, J=9.3 Hz, 1H), 4.06 (ddd, J=10.8, 6.4, 3.2 Hz, 1H), 3.76 (ddd, J=10.7, 7.0, 3.3 Hz, 1H), 3.40 (s, 3H), 2.98 (ddd, J=12.8, 7.1, 0.2 Hz, 1H), 2.82 (ddd, J=12.8, 6.4, 3.3 Hz, 1H), 2.53 (s, 3H), 2.43-2.33 (m, 2H), 2.04-2.00 (m, 1H), 1.88 (ddd, J=14.2, 6.6, 3.5 Hz, 1H), 1.75 (dq, J=13.6, 3.7 Hz, 1H), 1.69 (dt, J=13.4, 3.4 Hz, 1H), 1.55 (dd, J=13.8, 4.6 Hz, 1H), 1.47 (ddd, J=13.7, 11.4, 5.0 Hz, 1H), 1.41 (s, 3H), 1.35-1.22 (m, 2H), 1.03-0.97 (m, 1H), 0.95 (d, J=6.2 Hz, 3H), 0.89 (d, J=7.1 Hz, 3H). ¹³C NMR (151 MHz, CDCl₃) δ 104.31, 100.56, 91.18, 80.31, 67.65, 51.53, 51.00, 45.28, 37.33, 36.21, 35.35, 34.15, 32.56, 26.01, 24.64, 22.10, 20.22, 12.56. HR-MS (positive mode): calculated for [M+H]⁺ 342.2275; Found 342.2277.

Characterization of compound 9: ¹H NMR (600 MHz, Acetonitrile-d₃) δ 7.73 (dd, J=8.6, 2.0 Hz, 2H), 7.34-7.24 (m, 2H), 5.41 (s, 1H), 4.55 (dd, J=9.2, 2.8 Hz, 1H), 4.01 (ddt, J=31.8, 10.4, 5.1 Hz, 1H), 3.76-3.63 (m, 2H), 3.59-3.52 (m, 1H), 3.16 (s, 1.5H), 3.03 (s, 1.5H), 2.29 (td, J=14.0, 4.0 Hz, 2H), 2.05 (ddd, J=14.6, 5.0, 3.1 Hz, 1H), 1.91 (ddt, J=13.8, 6.9, 3.6 Hz, 1H), 1.77-1.65 (m, 2H), 1.54 (m, 1H), 1.49-1.43 (m, 2H), 1.32 (3H, s), 1.24 (m, 2H), 1.03 (m, 1H), 0.96 (d, J=6.4 Hz, 3H), 0.88 (dd, J=14.6, 7.1 Hz, 3H). ¹³C NMR (151 MHz, CD₃CN) δ 154.99, 154.93, 154.52, 154.44, 138.24, 131.43, 129.50, 129.40, 128.80, 123.91, 123.87, 104 37, 104.35, 100.73, 100.56, 91.53, 80.97, 67.04, 66.57, 52.21, 49.50, 49.47, 45.86, 45.84, 37.55, 36.68, 35.64, 35.61, 34.54, 33.32, 33.30, 25.80, 25.12, 22.33, 20.12, 12.51. ¹⁹F NMR (565 MHz, CD3CN) δ −44.26. HR-MS (positive mode): calculated for [M+H]⁺ 562.2081; Found 562.2082.

### Results

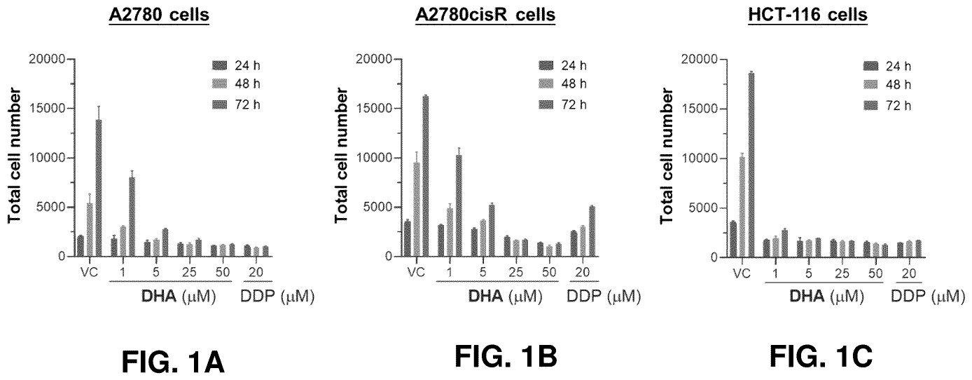

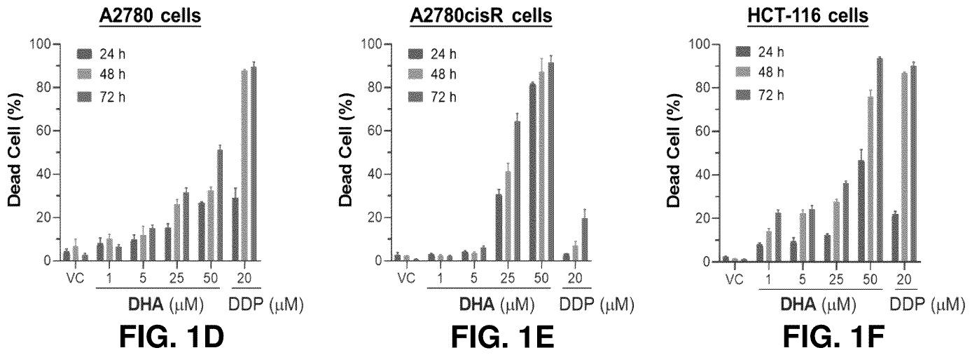

DHA displayed more particularly efficient in vitro cytotoxicity against A2780 ovarian cancer cells (IC₅₀=0.42 μM) and HCT-116 colon cancer cells (IC₅₀=0.74 μM) compared to MHCC-97L liver cancer cells (IC₅₀=33.03 μM) after 72 h treatment. More importantly, unlike cisplatin, which showed an ˜30-fold higher IC₅₀ value in DDP-resistant ovarian cancer cells (A2780cis, IC₅₀=12.06 μM) than that in cisplatin-sensitive A2780 cells (IC₅₀=0.39 M), DHA exhibited comparable cytotoxicity in A2780 and A2780cisR cells. These results demonstrated that DHA can overcome cisplatin resistance in ovarian cancer cells (Table 1). Further, high-content analysis by counting cell nuclei in the image (cells with Hoechst 33342 staining) and measuring cell membrane disruption (by propidium iodide staining) were performed to determine the total number and proportion of dead cells, respectively. As shown in FIGS. 1A-1C, DHA significantly inhibited the proliferation of A2780 and HCT-116 cancer cells at 5 μM. 50 M DHA was found to induce death in 51.4% of A2780 cells (FIG. 1D). The cell death rate increased to 91.7% of A2780cis cells and 93.8% of HCT-116 cells after treated with 50 M DHA (FIGS. 1E and 1F).

The in vitro cytotoxicity of various DHA derivatives against different cancer cells was also evaluated. As shown in Table 1, the DHA derivatives exhibited in vitro cytotoxicity against ovarian and colon cancer cell lines after 72 h treatment, with the best IC₅₀ values down to 0.32 μM.

TABLE 1
IC₅₀ values of DHA and its derivatives against different
cancer cell lines as determined by MTT (72 h, μM)

| | | Ovarian | | Lung | | |
|---|---|---|---|---|---|---|
| | Ovarian | cancer | Colon | cancer | Liver | |
| | cancer | CisPt resist. | cancer | NCI- | MHCC- | Cervical |
| Compounds | A2780 | A2780CisR | HCT-116 | H460 | 97L | Hela |
| Artemisinin | >100 | >100 | >100 | >100 | >100 | >100 |
| (ART) | | | | | | |
| DHA | 0.42 ± 0.02 | 0.80 ± 0.14 | 0.74 ± 0.04 | 5.42 ± 0.45 | 33.03 ± 3.59 | 32.92 ± 3.64 |
| 1 | 0.32 ± 0.01 | 0.39 ± 0.05 | 0.34 ± 0.01 | 0.41 ± 0.01 | 27.50 ± 1.17 | 42.27 ± 2.64 |
| 2 | 2.18 ± 0.24 | 5.44 ± 1.84 | 8.51 ± 1.74 | 56.52 ± 2.07 | 64.94 ± 4.38 | >100 |
| 3 | 0.44 ± 0.04 | >100 | 1.29 ± 0.05 | >100 | >100 | >100 |
| 4 | 2.96 ± 1.27 | 9.71 ± 0.90 | 6.89 ± 1.03 | >100 | 47.31 ± 9.72 | >100 |
| 5 | 0.34 ± 0.02 | 0.37 ± 0.01 | 0.38 ± 0.03 | 15.02 ± 0.96 | 25.17 ± 1.93 | 30.63 ± 1.27 |
| 6 | 34.67 ± 5.98 | >100 | >100 | >100 | >100 | >100 |
| 7 | 0.60 ± 0.22 | 7.56 ± 1.22 | 12.77 ± 1.29 | 3.65 ± 0.99 | 31.92 ± 1.88 | 55.02 ± 4.54 |
| 8 | 7.61 ± 0.73 | >100 | >100 | >100 | 68.76 ± 2.41 | >100 |
| 9 | 0.33 ± 0.01 | 0.35 ± 0.01 | 0.41 ± 0.02 | 33.20 ± 4.44 | 35.61 ± 1.25 | >100 |
| Cisplatin | 0.39 ± 0.04 | 12.06 ± 0.42 | 3.10 ± 0.29 | 11.67 ± 0.97 | 13.80 ± 0.34 | 4.39 ± 0.22 |

### Example 2. Dihydroartemisinin (“DHA”) Derivatives Show Anticancer Activity

The carbamodithioate group compound 11 showed cytotoxicity against the ovarian cancer cell lines A2780 (IC₅₀=40 nM) and A2780CisR (IC₅₀=60 nM), as well as the colon cancer cell line HCT-116 (IC₅₀=70 nM). Consequently, compound 11 was selected for further investigation as a chemotherapeutic payload for targeted therapy. Next, the methyl group in compound 4 was replaced with a hexanol group, yielding compound 10, which was subsequently transformed into the target carbamodithioate compound 12. Cytotoxicity studies revealed that compound 12 maintained potent activity against ovarian (A2780, IC50=70 nM; A2780CisR, IC50=140 nM) and colon (HCT-116, IC50=110 nM) cancer cell lines. This indicated that the addition of the branched hexanol group retains cytotoxicity, establishing compound 12 as a payload for targeted therapy. Further biological studies were conducted, and the results are presented below.

### Materials and Methods

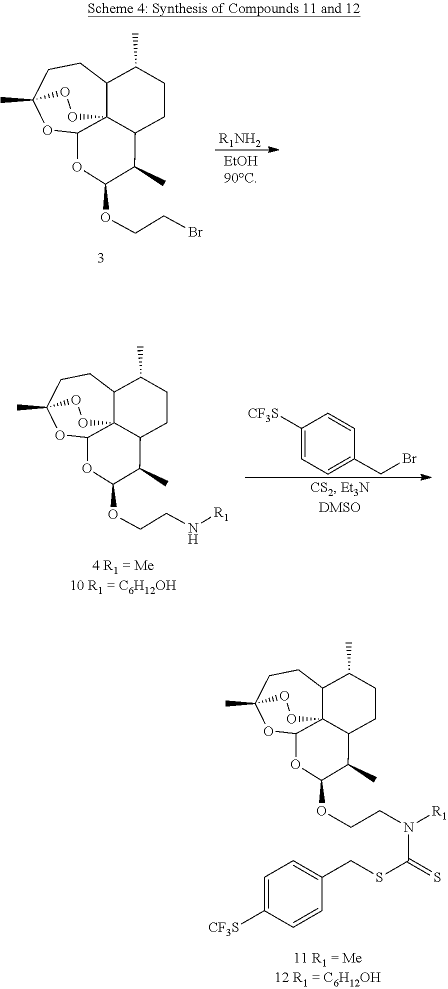

Amine 10: To a solution of bromo compound 3 (3.80 g, 9.74 mmol) in EtOH (25 mL) was added 6-amino-1-hexanol (1.71 g, 14.61 mmol) in seal-tube. The reaction was heated at 80° C. for 16 h. The reaction mixture was cooled to room temperature upon completion and concentrated in vacuo. Purification by silica gel flash chromatography afforded the desired amine compound 10 (2.82 g, 68%).

Carbamodithioate 11: To the solution of methylamine 4 (120 mg, 0.35 mmol) in DMSO (5 mL) and triethylamine (100 μL, 0.70 mmol) and carbon disulfide (21 μL, 1.05 mmol) were added. The mixture is stirred at room temperature for 30 minutes and 4-(Trifluoromethylthio)benzyl bromide (188 mg, 0.70 mmol) was added dropwise. The reaction mixture was stirred at room temperature for 16 h. The reaction mixture was diluted with EtOAc, then washed with brine, the organic layer was dried with Na₂SO₄, filtered and evaporated in vacuo to afford crude product, which was purified by silica gel flash chromatography to afford the desired carbamodithioate compound 11 (189 mg, 89%).

Carbamodithioate 12: To the solution of amine 10 (800 mg, 1.87 mmol) in DMSO (5 mL) and triethylamine (520 μL, 3.74 mmol) and carbon disulfide (200 μL, 5.62 mmol) were added. The mixture is stirred at room temperature for 30 minutes and 4-(Trifluoromethylthio)benzyl bromide (1.00 g, 3.74 mmol) was added dropwise. The reaction mixture was stirred at room temperature for 16 h. The reaction mixture was diluted with EtOAc, then washed with brine, the organic layer was dried with Na₂SO₄, filtered and evaporated in vacuo to afford crude product, which was purified by silica gel flash chromatography to afford the desired carbamodithioate compound 12 (1.11 g, 86%).

### Characterization of Compound 10:

¹H NMR (600 MHz, Chloroform-d) δ 5.43 (s, 1H), 4.82 (d, J=3.6 Hz, 1H), 4.18 (dt, J=11.7, 5.0 Hz, 1H), 3.88 (dt, J=11.5, 5.6 Hz, 1H), 3.59 (t, J=6.4 Hz, 2H), 3.22 (t, J=5.3 Hz, 2H), 3.10-2.97 (m, 2H), 2.67-2.60 (m, 1H), 2.38-2.28 (m, 1H), 2.00 (ddd, J=14.6, 4.8, 2.91 Hz, 1H), 1.85 (td, J=6.8, 3.1 Hz, 3H), 1.77-1.72 (m, 1H), 1.71-1.60 (m, 2H), 1.55 (t, J=6.6 Hz, 2H), 1.49-1.33 (m, 10H), 1.21 (td, J=11.4, 6.7 Hz, 1H), 0.96-0.82 (m, 7H).

¹³C NMR (151 MHz, CDCl₃) δ 104.19, 102.58, 88.01, 80.83, 77.21, 77.00, 76.79, 64.31, 62.03, 52.34, 48.20, 47.22, 44.04, 37.16, 36.23, 34.43, 32.01, 30.56, 26.17, 26.12, 25.95, 25.06, 24.56, 24.44, 20.26, 13.03.

HR-MS (positive mode): calculated for [M+H]⁺ 428.3007; Found 428.3010.

### Characterization of Compound 11:

¹H NMR (600 MHz, Acetonitrile-d₃) δ 7.67 (m, 2H), 7.56 (m, 2H), 5.35 (d, J=17.1 Hz, 1H), 4.73-4.64 (m, 2H), 4.59 (dd, J=17.8, 14.0 Hz, 1H), 4.43 (ddd, J=13.7, 6.7, 4.3 Hz, 0.6H), 4.19 (ddd, J=13.7, 6.3, 4.2 Hz, 0.6H), 4.11 (ddd, J=10.6, 5.2, 3.2 Hz, 1H), 4.05 (m, 0.4H), 3.94 (m, 0.4H), 3.63 (m, 1H), 3.44 (d, J=57.3 Hz, 3H), 2.48 (m, 1H), 2.27 (m, 1H), 2.05 (m, 1H), 1.89 (m, 1H), 1.84-1.66 (m, 2H), 1.65-1.51 (m, 2H), 1.50-1.27 (m, 3H), 1.25-1.15 (m, 2H), 0.96-0.84 (m, 7H).

¹³C NMR (151 MHz, CD₃CN) δ 196.66, 141.83, 136.96, 131.14, 131.06, 122.91, 104.26, 102.30, 102.18, 88.21, 81.36, 64.89, 56.96, 54.15, 53.05, 44.74, 44.70, 44.10, 41.34, 41.05, 40.91, 37.69, 37.57, 36.71, 36.69, 34.97, 34.92, 31.34, 31.31, 25.84, 25.13, 25.09, 24.93, 24.88, 20.24, 20.20, 12.87.

¹⁹F NMR (565 MHz, CD₃CN) δ −43.91.

HR-MS (positive mode): calculated for [M+H]⁺ 608.1780; Found 608.1777.

### Characterization of Compound 12:

¹H NMR (600 MHz, Acetonitrile-d₃) δ 7.60 (d, J=8.2 Hz, 2H), 7.48 (d, J=8.2 Hz, 2H), 5.29 (d, J=8.1 Hz, 1H), 4.64 (s, 1H), 4.62-4.51 (m, 2H), 4.25-4.11 (m, 2H), 3.94 (m, 0.6H), 3.75 (m, 1.4H), 3.63-3.50 (m, 1H), 3.42 (m, 2H), 2.44 (m, 2H), 2.21 (m, 1H), 1.99 (m, 1H), 1.82 (ddt, J=10.4, 7.0, 3.5 Hz, 1H), 1.69 (m, 4H), 1.60-1.40 (m, 3H), 1.39-1.21 (m, 9H), 1.14 (m, 1H), 0.90-0.79 (m, 7H).

¹³C NMR (151 MHz, CD₃CN) δ 195.98, 195.83, 141.86, 141.77, 136.96, 131.46, 131.13, 131.04, 129.43, 122.90, 104.27, 102.29, 102.14, 88.21, 88.20, 81.35, 65.16, 64.89, 62.02, 61.94, 56.11, 54.98, 54.64, 53.03, 44.73, 44.70, 41.00, 40.71, 37.73, 37.65, 36.70, 34.94, 34.90, 33.04, 32.97, 31.36, 31.33, 27.31, 26.83, 26.75, 26.14, 25.84, 25.72, 25.12, 25.11, 24.98, 2022, 20.20, 12.89.

¹⁹F NMR (565 MHz, CD₃CN) δ −42.71

HR-MS (positive mode): calculated for [M+H]⁺ 716.2232; Found 716.2241.

### Results

Compounds 11 and 12 exhibited potent in vitro cytotoxicity against these cancer cells after 72 h of treatment, with IC₅₀ values ranging from 0.04 to 17.84 μM, which is generally one to two orders of magnitude lower than the IC₅₀ values of DHA treatment. Furthermore, 11 and 12 exhibited selective cytotoxicity against cancer cells, while the cytotoxicity induced in normal fibroblasts (CCD-19Lu) was attenuated. The IC₅₀ value in CCD-19Lu treated with 11 and 12 was about 100 to 200-fold higher than those in lung cancer cells treated with 11 and 12.

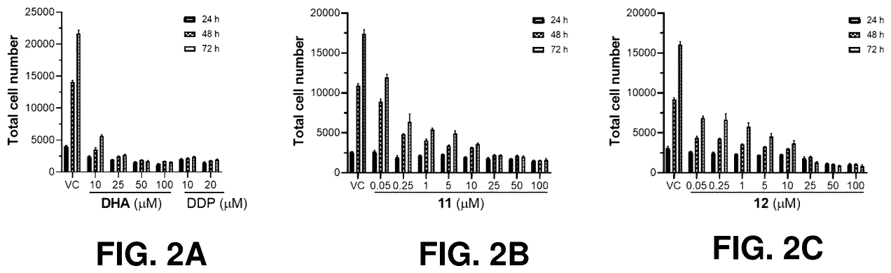

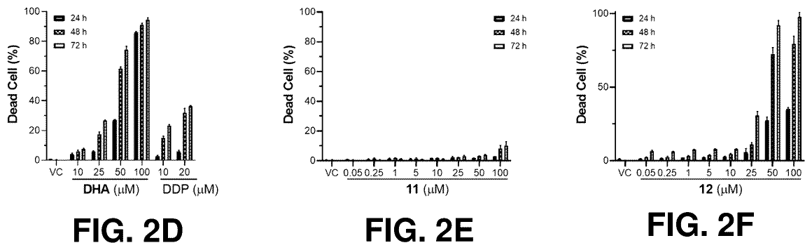

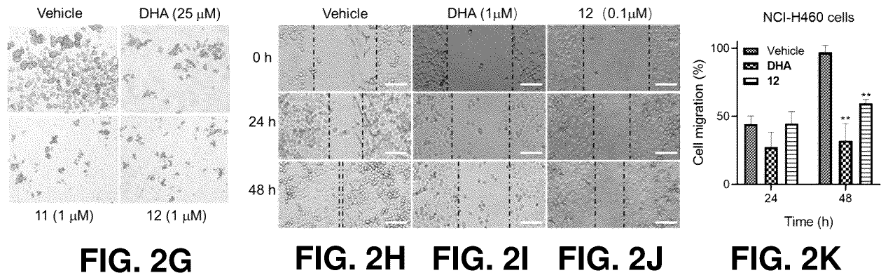

In addition, high-content analysis was performed by counting cell nuclei in the image (cells with Hoechst 33342 staining) and measuring cell membrane disruption (by propidium iodide staining) to determine the total number and proportion of dead cells, respectively, after DHA treatment. Results showed that DHA significantly inhibited the proliferation of NCI-H460 cancer cells at concentrations at 10 μM (FIGS. 2A-2F). DHA at 100 μM markedly induced cancer cell death, killing 94.5% of the cells within 72 h of treatment. For comparison, 20 M DDP was found to induce death in 36.4% of NCI-H460 cancer cells. ART-3 at 100 μM induced 4.1% cell death, but 12 at 100 μM markedly induced cancer cell death, killing 97.8% of the cells within 72 h of treatment. The effect of DHA and its derivatives on cancer stem cell expansion was assessed through a tumor-sphere formation assay, and the results showed that 11 and 12 have more inhibitory effect on tumor-sphere growth and reduced the number and size of formed tumor spheres compared with 25 μM DHA (FIG. 2G).

The effect of DHA and its derivatives on cell motility was studied using a wound-healing scratch assay and it showed that cell migration to the wound area was inhibited after 48-h treatment with 1 μM DHA or 11 and 12 at 0.1 μM have a stronger inhibitory effect on cell migration. (FIGS. 2H-2K).

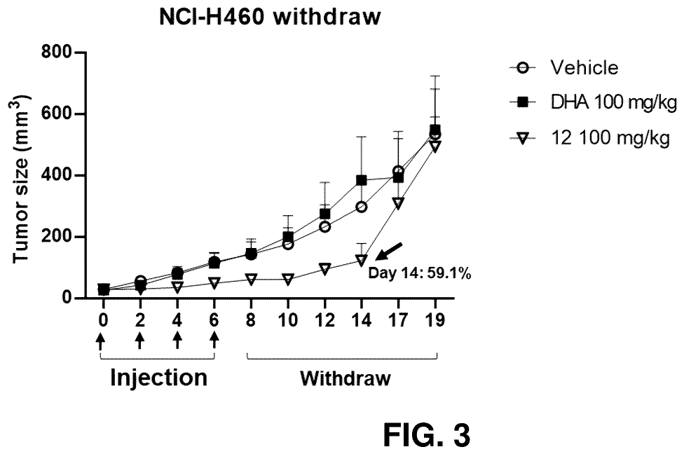

In vivo antitumor experiments were conducted on nude mice carrying human lung cancer NCI—H460 xenografts. Treatment of mice with DHA (100 mg/kg, twice weekly Intraperitoneally) and 12 (100 mg/kg, twice weekly intravenously) resulted in a reduction in tumor volume of 59.1% of 12 compared to DHA is not effective for NCI—H460 xenografts. And effect of 12 can last for around one week since the last injection (FIG. 3).

TABLE 2
Cell Viability Assays of DHA and its derivatives and cisplatin in a panel of human cancer cells and normal cells

| | IC₅₀ (μM, 72 h) | | | | | | |
|---|---|---|---|---|---|---|---|
| | | Ovarian | | | Lung | | |
| | Ovarian | cancer CisPt | Colon | Lung | Normal | | |
| | cancer | resist. | cancer | cancer | fibroblast | Liver | Cervical |
| Compunds | A2780 | A2780CisR | HCT-116 | NCI-H460 | CCD-19Lu | MHCC-97L | Hela |
| ART | >100 | >100 | >100 | >100 | >100 | >100 | >100 |
| DHA | 0.42 ± 0.02 | 0.80 ± 0.14 | 0.74 ± 0.04 | 5.42 ± 0.45 | 64.26 ± 6.31 | 33.03 ± 3.59 | 32.92 ± 3.64 |
| 11 | 0.04 ± 0.002 | 0.06 ± 0.002 | 0.07 ± 0.01 | 0.15 ± 0.01 | 18.12 ± 1.59 | 17.84 ± 5.38 | 0.40 ± 0.01 |
| 12 | 0.07 ± 0.002 | 0.14 ± 0.01 | 0.11 ± 0.01 | 0.16 ± 0.01 | 33.57 ± 1.83 | 4.36 ± 0.23 | 0.67 ± 0.03 |
| Cisplatin | 0.39 ± 0.04 | 12.06 ± 0.42 | 3.10 ± 0.29 | 11.67 ± 0.97 | 60.80 ± 2.47 | 15.33 ± 1.41 | 5.37 ± 0.75 |

### REFERENCES

Huang, J.; Shi, Y.; Tan, S.; Li, M. CN108264454A, 2018.
R. K. Haynes, et al., European Journal of Organic Chemistry, 2002, 1, 113-132.

We claim:

1. A compound having the structure of: 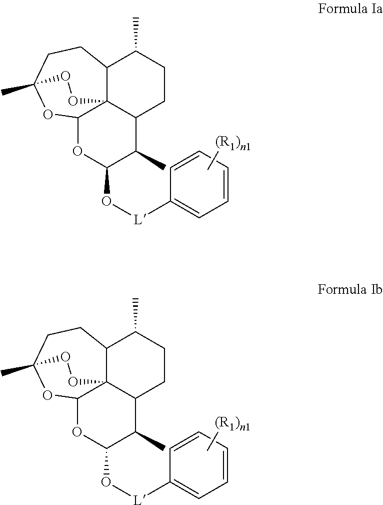
wherein:
(i) R₁ is an electron withdrawing group;
(ii) n1 is an integer from 0 to 5, from 1 to 5, from 1 to 4, from 1 to 3, or 1 or 2, such as 1; and
(iii) L′ is 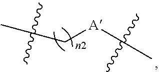
(a) n2 is an integer from 1 to 6, from 1 to 5, from 1 to 4, from 1 to 3, or 1 or 2;
(b) A′ is a bond, 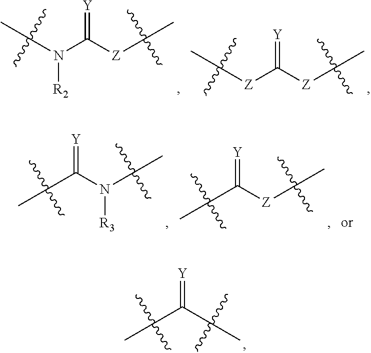
each occurrence of Y and Z are independently O or S, optionally A′ is a bond, 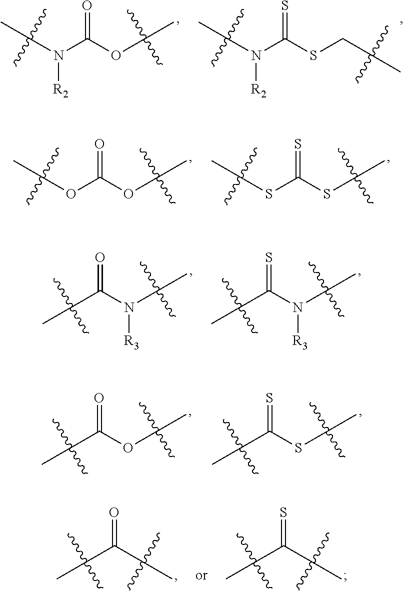
and
(c) R₂ and R₃ are independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

2. The compound of claim 1, having the structure of: 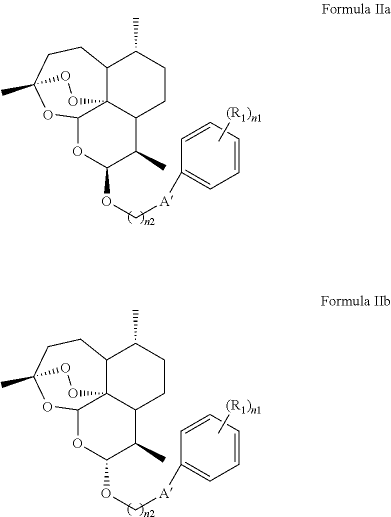
wherein:
(i) R₁ is an electron withdrawing group;
(ii) n1 is an integer from 0 to 5, from 1 to 5, from 1 to 4, from 1 to 3, or 1 or 2, such as 1;
(iii) n2 is an integer from 1 to 6, from 1 to 5, from 1 to 4, from 1 to 3, or 1 or 2; and
(iv) A′ is a bond, 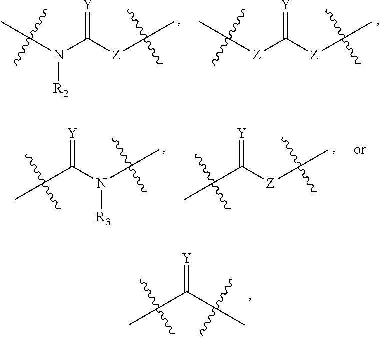
each occurrence of Y and Z are independently O or S, optionally A′ is a bond, 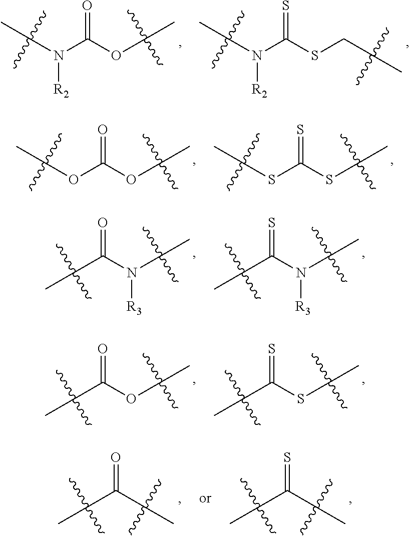
R₂ and R₃ are independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

3. The compound of claim 1, A′ is a bond, 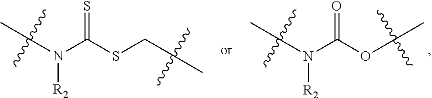
wherein R₂ is hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

4. The compound of claim 1, having a structure of any one of Formula IIIa-IIId: 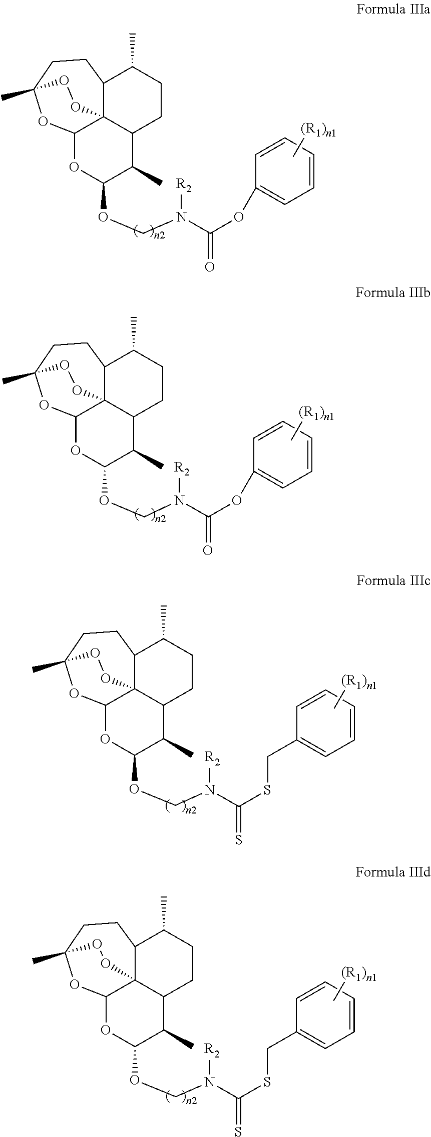
wherein:
(i) R₁ is an electron withdrawing group;
(ii) n1 is an integer from 0 to 5, from 1 to 5, from 1 to 4, from 1 to 3, or 1 or 2, such as 1;
(iii) n2 is an integer from 1 to 6, from 1 to 5, from 1 to 4, from 1 to 3, or 1 or 2; and
(iv) R₂ is independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester).

5. The compound of claim 1, wherein R₂ and R₃ are independently a methyl, an ethyl, a hexyl, or a hexyl substituted with a hydroxyl group.

6. The compound of claim 1, wherein A′ is a bond.

7. The compound of claim 1, wherein n1 and n2 are independently 1 or 2.

8. The compound of claim 1, wherein R₁ is a halide, an alkyl halide, a (trifluoromethyl)sulfanyl, a (trifluoromethyl)sulfinyl, a (trifluoromethyl)sulfonyl, a nitro, a nitrile, an aldehyde, a ketone, a carboxyl, a sulfonyl, a sulfinyl, or a sulfino.

9. The compound of claim 1, wherein R₁ is a halide or (trifluoromethyl)sulfanyl.

10. The compound of claim 1, having any one of the structures of:

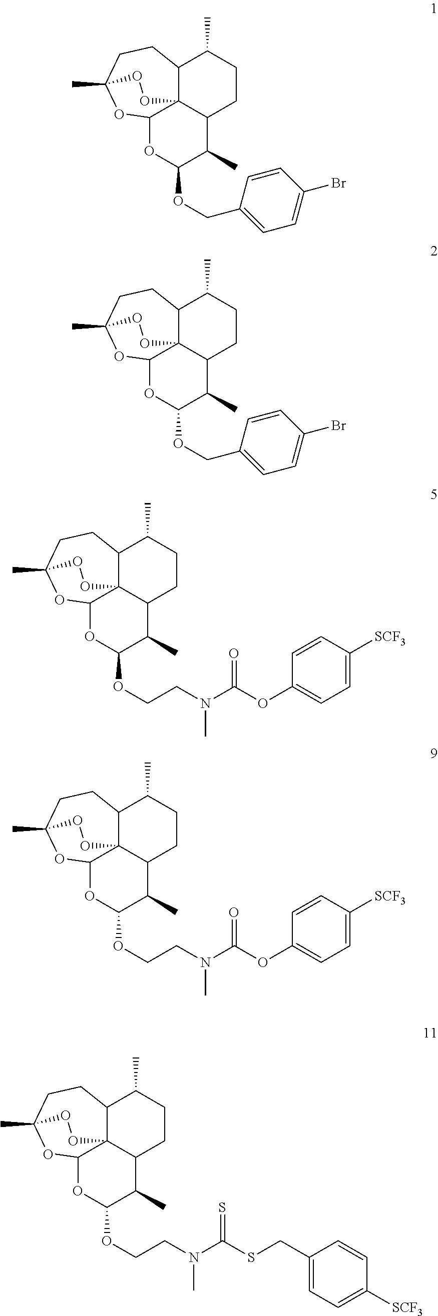

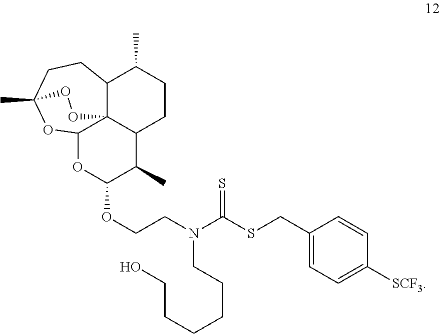

11. A pharmaceutical composition comprising one or more the compound(s) of claim 1, and a pharmaceutically acceptable carrier and/or excipient.

12. The pharmaceutical composition of claim 11, wherein the one or more compounds are in an effective amount to treat one or more symptoms associated with a cancer in a subject.

13. The pharmaceutical composition of claim 11, further comprising a second active agent that is different from the compound(s), optionally wherein the second active agent is an anticancer agent.

14. A method for treating a cancer in a subject comprising:
(i) administering to the subject the pharmaceutical formulation of claim 11, wherein step (i) occurs one or more times.

15. The method of claim 14, wherein the method comprises only a single administration of the pharmaceutical formulation, wherein following the administration step, an effective amount of the compound(s) to treat one or more symptoms associated with the cancer in the subject is administered to the subject, or
wherein the method comprises more than one step of administering to the subject the pharmaceutical formulation, wherein following all of the administration steps, an effective amount of the compound(s) to treat one or more symptoms associated with the cancer in the subject is administered to the subject.

16. The method of claim 14, wherein the cancer is a Cisplatin resistant cancer, such as Cisplatin resistant ovarian cancer.

17. The method of claim 14, wherein the cancer is ovarian cancer or colon cancer.

18. A method for synthesizing DHA derivatives having a structure of Formula IVa, Formula IVb, or Formula IVc: 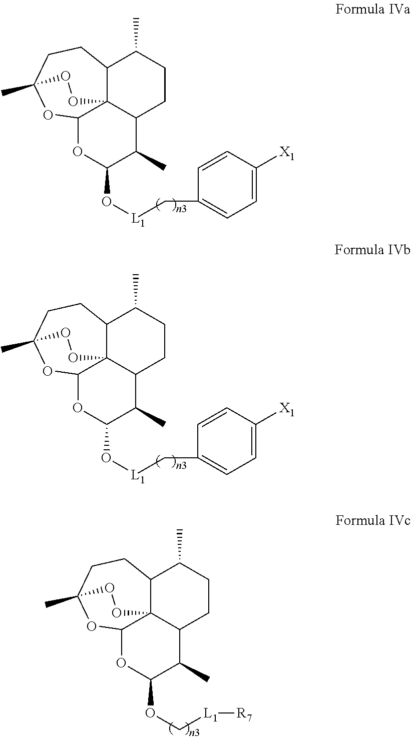
wherein: (i) X₁ is a halide (e.g., bromide), hydroxyl, or thiol; (ii) n3 is an integer from 0 to 5, from 1 to 5, from 1 to 4, from 1 to 3, or 1 or 2, such as 1; (iii) L₁ is a bond, 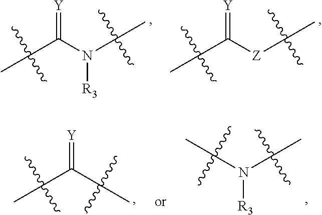
each occurrence of Y and Z are independently O or S (such as 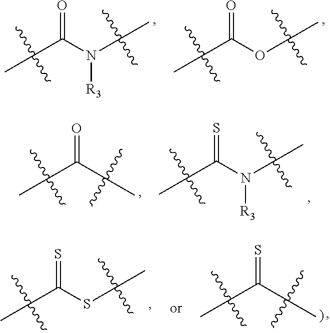
R₃ is hydrogen or an alkyl (such as an unsubstituted C1-C6 alkyl, an unsubstituted C1-C5 alkyl, an unsubstituted C1-C4 alkyl, an unsubstituted C1-C3 alkyl, a methyl, or an ethyl); and (iv) R₇ is an alkyl (e.g., methyl or ethyl) or 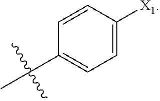
the method comprising: (a) maintaining a first reaction mixture at a temperature of 0° C. to room temperature for a time period of 2 hours to 15 hours, from 5 hours to 15 hours, or from 5 hours to 10 hours to form a first product comprising the DHA derivatives of Formula IVa and Formula IVb,
wherein the first reaction mixture comprises DHA, a first reactant having the structure of Formula V, a catalyst, and an organic solvent, 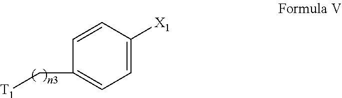
wherein: X₁ and n3 are as defined for Formula IVa and Formula IVb; and T₁ is hydroxyl, 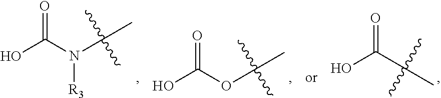
R₃ is as defined above for Formula IVa, IVb, and IVc.

19. A method for synthesizing a DHA derivative having the structure of Formula IIa or Formula IIb, 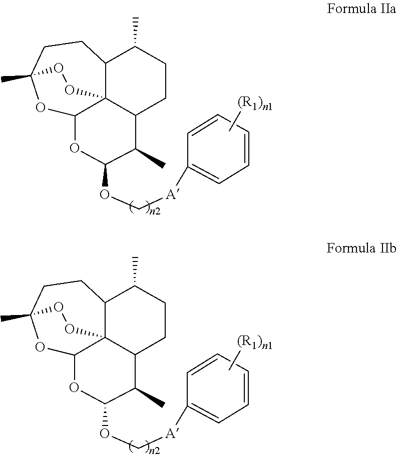
wherein: (i) R₁ is an electron withdrawing group; (ii) n1 is an integer from 0 to 5, from 1 to 5, from 1 to 4, from 1 to 3, or 1 or 2, such as 1; (iii) n2 is an integer from 1 to 6, from 1 to 5, from 1 to 4, from 1 to 3, or 1 or 2; and (iv) A′ is a bond, 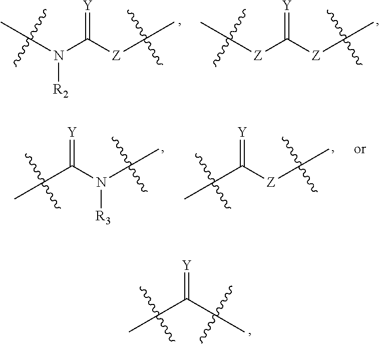
each occurrence of Y and Z are independently O or S, optionally A′ is a bond, 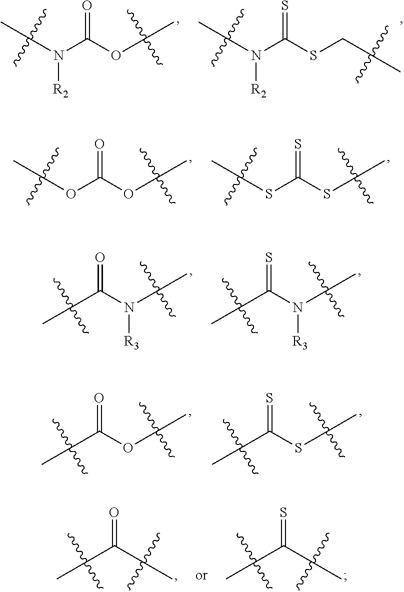
wherein R₂ and R₃ are independently hydrogen or an alkyl (such as an unsubstituted C1-C6 alkyl, a substituted C1-C6 alkyl, an unsubstituted C1-C5 alkyl, a substituted C1-C5 alkyl, an unsubstituted C1-C4 alkyl, a substituted C1-C4 alkyl, an unsubstituted C1-C3 alkyl, a substituted C1-C3 alkyl, for example, a methyl, an ethyl, an n-propyl, an isopropyl, an n-butyl, an iso-butyl, a tert-butyl, an n-pentyl, an iso-pentyl, a tert-pentyl, an n-hexyl, an iso-hexyl, or a tert-hexyl, each optionally substituted with a hydroxyl, a thiol, an alkoxy, a carboxylic acid, a ketone, an aldehyde, or an ester),
the method comprising: (b) maintaining a second reaction mixture at a temperature of −10° C. to room temperature for a time period of 2 hours to 15 hours, from 5 hours to 15 hours, or from 5 hours to 10 hours to form an intermediate product comprising an intermediate of Formula VI or Formula VIII; and (c) converting the intermediate of Formula VI or Formula VIII to a second product comprising the DHA derivative of Formula IIa or Formula IIb, 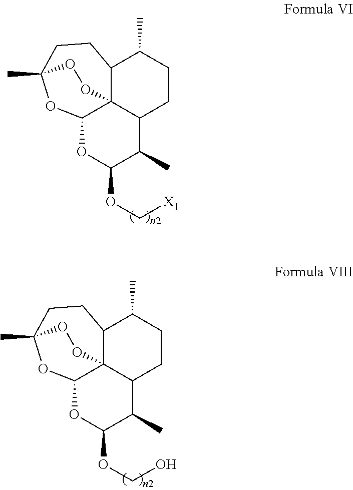
wherein the second reaction mixture comprises DHA, a second reactant having the structure of Formula VII, a catalyst, and an organic solvent, 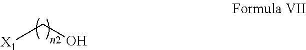
wherein n2 is an integer from 1 to 6, from 1 to 5, from 1 to 4, from 1 to 3, or 1 or 2; and X₁ is a halide or hydroxyl.

20. The method of claim 19 for synthesizing a DHA derivative having the structure of Formula IIb, wherein X₁ is hydroxyl.
